(19) United States
(12) Patent Application Publication
(10) Pub. No.: US 2025/0099572 A1
(43) Pub. Date: Mar. 27, 2025

(54) VACCINE COMPOSITIONS

(71) Applicant: Monash University, Clayton, Victoria (AU)

(72) Inventors: Hareth Basim Ali AL-WASSITI, Clayton, Victoria (AU); Colin William POUTON, Clayton, Victoria (AU); Stewart Alastair FABB, Clayton, Victoria (AU)

(73) Assignee: Monash University, Clayton, Victoria (AU)

(21) Appl. No.: 18/291,812

(22) PCT Filed: Aug. 17, 2022
(86) PCT No.: PCT/AU2022/050912

§ 371 Date: Jan. 24, 2024

(30) Foreign Application Priority Data
Aug. 17, 2021 (AU) 2021902566

(51) Int. Cl. A61K 39/215; A61K 39/00; A61P 37/04; C07K 14/005

(52) U.S. Cl. CPC A61K 39/215; A61P 37/04; C07K 14/005; A61K 2039/53; A61K 2039/6018; C07K 2319/02

(57) ABSTRACT

The invention relates to vaccine compositions for inducing an immune response to a coronavirus in a subject, and uses thereof. In particular, the vaccine comprises of a chimeric or fusion protein comprising a) a N-terminal secretion signal peptide; b) an amino acid sequence of the receptor binding domain (RBD) of a spike protein of a coronavirus; and c) a C-terminal domain comprising a transmembrane region and a cytoplasmic region. In a preferred embodiment, the signal peptide, RBD, transmembrane region, and cytoplasmic region are derived from SARS-CoV-2, and that the vaccine composition is formulated as a lipid nanoparticle (LNP).

### FIELD OF THE INVENTION

The present invention relates to vaccine compositions for inducing an immune response to a coronavirus in a subject, and uses thereof.

### RELATED APPLICATION

This application claims priority from Australian provisional application AU 2021902566, the entire contents of which are hereby incorporated by reference.

### BACKGROUND OF THE INVENTION

The ongoing global pandemic caused by a novel coronavirus, SARS-CoV-2, has presented an urgent need for the development for new and effective vaccines.

With a death toll surpassing 500,000 in the United States alone, containing the pandemic is predicated on achieving herd immunity on a global scale. This implies that at least 70-80% of the population must achieve active immunity against the severe acute respiratory syndrome coronavirus 2 (SARS-CoV-2), either as a result of a previous COVID-19 infection or by vaccination against SARS-CoV-2.

Coronaviruses, belong to the Coronaviridae family in the Nidovirales order, are minute in size (65-125 nm in diameter) and contain a single-stranded RNA as a nucleic material, size ranging from 26 to 32 kbs in length. The subgroups of coronaviruses family are alpha (α), beta (γ), gamma (γ) and delta (δ) coronavirus. The beta-coronaviruses are of the greatest clinical importance concerning humans. These include OC43 and HKU1 (which can cause the common cold) which are a beta-coronavirus of lineage A. Beta-coronaviruses of Lineage B include the severe acute respiratory syndrome coronaviruses SARS-CoV and SARS-CoV-2 (which causes the disease COVID-19). Middle East respiratory syndrome coronavirus (MERS-CoV) is a beta-coronavirus from lineage C. These viruses cause acute lung injury (ALI) and acute respiratory distress syndrome (ARDS) which leads to pulmonary failure and death.

Prior to 2020, no vaccine directed to a coronavirus had been successfully developed. Since then, a number of vaccines have been developed, directed to SARS-CoV-2.

A successful vaccination campaign is contingent on widespread access to the vaccine under appropriate storage conditions, deployment of a sufficient number of vaccinators, and the willingness of the population to be vaccinated. Moreover, the rapid emergence of variants of SARS-CoV-2 has highlighted the importance of vaccines which are effective in inducing an immune response that is effective for protecting against variants as they inevitably arise. Further still, there is a need for vaccines which can be used to induce an effective immune response (such as induction of neutralising antibodies, rather than the induction of potentially harmful non-neutralising antibodies).

There is a continued need for the development of vaccines which elicit a protective immune response against coronaviruses.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a polynucleotide encoding a chimeric or fusion protein comprising or consisting of:
a) an N-terminal secretion signal peptide;
b) an amino acid sequence of the receptor binding domain (RBD) of a coronavirus spike protein, or variant thereof; and
c) a C-terminal domain comprising a transmembrane region and optionally, a cytoplasmic region preferably, wherein the polynucleotide is capable of being translated in a mammalian cell.

In preferred embodiments of the invention, the polynucleotide is a messenger RNA (mRNA) molecule. The mRNA may further comprise a 5′ untranslated region (UTR) and a 3′ UTR. The mRNA may also comprise a 5′ cap analog, such as 7mG(5′)ppp(5′)NlmpNp. The mRNA may also comprise a Polyadenine (poly A) tail.

The mRNA may comprise a chemical modification. Examples of suitable chemical modification include a 1-methylpseudouridine modification or a 1-ethylpseudouridine modification or may comprise any chemical modification described herein.

In any embodiment of the invention, the polynucleotide is in the form of a codon optimised mRNA molecule, optionally depleted of uridine nucleosides. In any embodiment, the codon optimisations comprises conversion of codons encoding serine to UCG.

Preferably, the polynucleotide has a uridine content of less than about 50%, less than about 45%, less than about 40%, less than about 35%, less than about 30%, less than about 25%, less than about 20% or less than about 15%. In preferred embodiments, the polynucleotide has a uridine content of between about 15% and about 35%, preferably between about 15% and about 25%.

In preferred embodiments, the uridines in the polynucleotide are replaced with a chemical modification such as N-methyl-pseudouridine. Preferably, at least 25%, at least 30%, at least 35%, at leat 40%, at least 45%, at least 50%, at least 55%, at least 60%, at least 65%, at least 70%, at least 75%, at least 80%, at least 85%, at least 90%, at least 95%, or 100% of the uridine nucleosides are replaced with N-methyl-pseudouridine.

In further preferred embodiments of the invention, the polynucleotide has a total GC content of greater than about 25%, greater than about 30%, greater than about 35%, greater than about 40%, greater than about 45%, greater than about 50%, greater than about 55%, greater than about 60% or more.

In any embodiment, the N-terminal secretion signal peptide comprises any amino acid sequence which enables the chimeric or fusion protein to be processed by ribosomes bound to the rough endoplasmic reticulum (ER) of a cell, and thereby results in threading of the chimeric or fusion protein into the ER. From the ER, the chimeric or fusion protein is capable of being transported to the plasma membrane of the mammalian cell. It will be appreciated that the N-terminal secretion signal peptide is not intended for directing the chimeric or fusion protein to be secreted outside of the mammalian cell in which it is expressed.

In any embodiment, the N-terminal secretion signal peptide comprises the amino acid sequence of any secretion signal from a coronavirus. Preferably, the amino acid sequence is of a secretion peptide of any coronavirus spike protein. It will be appreciated that the N-terminal secretion peptide may be derived from a spike protein of the same coronavirus strain or from a different coronavirus strain, as the RBD sequence. For example, in one embodiment, the RBD sequence may be from a SARS-CoV-2 spike protein and the N-terminal secretion signal peptide may be from any SARS-CoV protein, optionally from SARS-CoV spike protein. In alternative embodiments, the RBD sequence may a SARS-CoV-2 spike protein RBD sequence and the N-terminal secretion signal peptide may also be from the SARS-CoV-2 spike protein.

In any embodiment of the invention, the N-terminal secretion signal peptide may comprise an amino acid sequence that is cleavable to enable cleavage of the RBD amino acid sequence from the secretion signal peptide following translation of the polynucleotide sequence. In any embodiment, the N-terminal secretion signal peptide does not comprise a cleavable sequence or the polynucleotide does not encode a cleavable sequence between the secretion signal peptide and the RBD.

The cleavable sequence may comprise an amino acid sequence of between 1 to 10 amino acids, preferably a sequence of no more than between 1 and 5 amino acids. In any embodiment, the cleavable linker sequence comprises a sequence that is susceptible to cleavage by a protease.

In preferred embodiments of the invention, the signal peptide comprises the sequence as set forth in SEQ ID NO: 4, or a sequence at least 80%, at least 85%, at least 90%, at least 95%, at least 96%, at least 97%, at least 98% or at least 99% identical thereto.

In any embodiment of the invention, the amino acid sequence of an RBD of a spike protein of a coronavirus is preferably an amino acid sequence of an RBD from a beta-coronavirus, more preferably a beta-coronavirus from Lineage B, such as SARS-CoV or SARS-CoV-2. Most preferably, the amino acid sequence is derived from the RBD from SARS-CoV-2 or a mutated form or variant thereof, including but not limited to the alpha, beta, epsilon, kappa, delta, delta-plus, lambda, gamma, mu or omicron variants. Alternatively, the amino acid sequence is of an RBD may be from a beta-coronavirus from Lineage C, optionally MERS-CoV.

In a preferred embodiment of the invention, the coronavirus is SARS-CoV-2 (or variant thereof) and accordingly, the polynucleotides of the invention encode a chimeric or fusion protein comprising b) an amino acid sequence of the RBD of a SARS-CoV-2 spike protein.

The chimeric or fusion protein (ie polypeptide) encoded by the polynucleotides of the invention, preferably comprises a C-terminal domain comprising a transmembrane domain and a cytoplasmic region. It will be appreciated that any amino acid sequence capable of threading through a lipid membrane, typically an amino acid sequence that forms an alpha helical structure, can be used. Transmembrane domain sequences are well known in the art, and can be determined using various bioinformatics tools, including SignalP and the like.

The transmembrane domain and cytoplasmic region may comprise the amino acid sequence of the transmembrane domain and cytoplasmic regions of any coronavirus protein, preferably from any coronavirus spike protein.

In certain embodiments, the transmembrane domain and cytoplasmic region are from a spike protein of the same coronavirus strain. For example, the transmembrane and cytoplasmic regions may both be from SARS-CoV-2 spike protein. Alternatively, the cytoplasmic region may be heterologous to the transmembrane domain. For example, the transmembrane region may be from SARS-CoV-2 spike protein, and the cytoplasmic region may be from a coronavirus that is not SARS-CoV-2. Alternatively, the transmembrane region may be from a spike protein that is not SARS-CoV-2, and the cytoplasmic region may be from SARS-CoV-2 spike protein.

In any embodiment of the invention, the chimeric or fusion protein comprises a flexible linker sequence between b) the amino acid sequence of the RBD and c) the C-terminal domain. The linker may comprise a series of amino acid residues, such as glycine, serine, glutamic acid and/or aspartic acid residues, which impart flexibility to the polypeptide. Examples of suitable linkers are well known to the skilled person and are described for example, in Chen et al., (2013) Advanced Drug Delivery Reviews, 65: 1357-1369.

In any embodiment the invention, the polypeptide encoded by the polynucleotide sequence does not comprise the full-length amino acid sequence of either the S1 or S2 domains of the spike protein of a coronavirus. In preferred embodiments, the polypeptide does not comprise one or more or all of the subdomains SD1 and SD2 of the S1 subunit of the spike protein of a coronavirus. In further preferred embodiments, the polypeptide does not comprise one or both of the HR1 and HR2 domains.

In optional embodiments, the polypeptide encoded by the polynucleotide sequence of the invention includes the N-terminal domain of the S1 domain of the spike protein from a coronavirus. In such embodiments, the polypeptide consists of a) an N-terminal secretion signal peptide and an amino acid sequence of an N-terminal domain of a coronavirus spike protein, b) an amino acid sequence of the RBD of a coronavirus spike protein and c) a C-terminal domain comprising a transmembrane region and optionally, a cytoplasmic region.

In alternative embodiments, the polypeptide encoded by the polynucleotides of the invention does not comprise the N-terminal domain of a coronavirus spike protein.

In particularly preferred embodiments of the invention, the coronavirus is SARS-CoV-2 (or variant thereof) and accordingly, there is provided a polynucleotide encoding a chimeric or fusion protein that comprises or consists of:
a) an N-terminal secretion signal peptide;
b) an amino acid sequence of the receptor binding domain (RBD) of SARS-CoV-2 spike protein, or variant thereof; and,
c) a C-terminal domain comprising a transmembrane region and optionally, a cytoplasmic region,
   preferably, wherein the polynucleotide is capable of being translated in a mammalian cell.

Preferably, the polynucleotide is in the form of an mRNA. Accordingly, in preferred embodiments of the invention, there is provided an mRNA encoding a chimeric or fusion protein that comprises or consists of:
a) an N-terminal secretion signal peptide;
b) an amino acid sequence of a receptor binding domain (RBD) of SARS-CoV-2 spike protein, or variant thereof; and optionally
c) a C-terminal domain comprising a transmembrane region and a cytoplasmic region.

The mRNA may further comprise a 5′ untranslated region (UTR) and a 3′ UTR. The mRNA may also comprise a 5′ cap analog, such as 7mG(5′)ppp(5′)NlmpNp. The mRNA may also comprise a polyA tail.

In any embodiment, the mRNA may comprise a chemical modification.

Examples of suitable chemical modification include a 1-methylpseudouridine modification or a 1-ethylpseudouridine modification or any other chemical modification described herein.

It will be appreciated that the SARS-CoV-2 spike RBD sequence may comprise the sequence of the originally described “ancestral” SARS-CoV-2 virus, for example, as set forth in SEQ ID NO: 9, or a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto. As used herein, the terms “wild-type” “WT” or “ancestral” may be used interchangeably to refer to the original SARS-CoV-2 strain of virus identied.

The present invention also contemplates polynucleotides that encode a chimeric or fusion protein that comprises the RBD of any SARS-CoV-2 variant or mutant. In certain embodiments, the variant RBD sequence may comprise the sequence of a SARS-CoV-2 alpha (UK) variant, a beta (South African), an epsilon (United States) variant, a kappa (Indian) variant, a delta (Indian) variant, a delta plus (Indian) variant, lambda variant or combinations thereof. Exemplary sequences of RBD-TM chimeric or fusion proteins comprising sequences of variant RBDs are exemplified herein in SEQ ID NOs: 15 (Beta), 18 (Delta), 21 (Delta plus), 24 (Omicron BA1) and 27 (Omicron BA2).

The RBD amino acid sequence may comprise the sequence of RBD having a variation of mutation relative to SEQ ID NO: 8 herein. The mutation or variation may be at any one or more of residues N191, E174, K107, L142, T168 or S167 of SEQ ID NO: 9 (equivalent to residues N501, E484, K417, L452, T478, K417 or S447 of SEQ ID NO: 1).

The mutation or variation may comprise one or more of N191Y, E174K, K107N, L142R, E174Q, T168K, K107N or S167N (according to SEQ ID NO: 9; equivalent to N501Y, E484K, K417N, L452R, E484Q, T478K, K417N or S447N according to SEQ ID NO: 1).

Preferably, the N-terminal secretion signal peptide comprises an amino acid sequence corresponding to the SARS-CoV-2 spike protein, although it will be appreciated that any N-terminal secretion signal peptide can be used, provided that the sequence enables the polypeptide to be thread into the ER. In certain examples, the signal peptide may be derived from a protein that is not a spike protein from a coronavirus. In certain examples, the signal peptide may be derived from the spike protein of a coronavirus that is not a SARS-CoV-2. Further still, the signal peptide may be derived from the spike protein of a SARS-CoV-2 that is variant to the SARS-CoV-2 from which the RBD amino acid sequence is derived.

In preferred embodiments of the invention, and wherein the coronavirus is SARS-CoV-2, the signal peptide may comprise the sequence as set forth in SEQ ID NO: 4, or a sequence at least 80%, at least 85%, at least 90%, at least 95%, at least 96%, at least 97%, at least 98% or at least 99% identical thereto.

Preferably, the C-terminal domain preferably comprises the amino acid sequence of the transmembrane domain of the spike protein of SARS-CoV-2. Accordingly, the C-terminal domain preferably comprises a transmembrane domain comprising the amino acid sequence as set forth in SEQ ID NO: 5 or 6, or a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto.

Furthermore, the C-terminal domain preferably comprises the amino acid sequence of the cytoplasmic region of the spike protein of SARS-CoV-2. Accordingly, the C-terminal domain preferably comprises a cytoplasmic region comprising the amino acid sequence as set forth in SEQ ID NO: 7, or a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto.

In certain embodiments, the transmembrane domain and cytoplasmic region are derived from the spike protein of SARS-CoV-2 (including variants and mutants thereof). In preferred embodiments, the transmembrane domain and cytoplasmic regions comprise amino acid sequences derived from the same SARS-CoV-2 strain or variant. In alternative embodiments, the transmembrane domain and cytoplasmic regions may be derived from different SARS-CoV-2 strains or variants. In particularly preferred embodiments, wherein the coronavirus is SARS-CoV-2, the C-terminal domain preferably comprises the amino acid sequence as set forth in SEQ ID NO: 8, or a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto.

In any embodiment of the invention, the polynucleotide (preferably an mRNA) encodes a chimeric or fusion protein comprising or consisting of the sequence set forth in any one of SEQ ID NOs: 3, 12, 15, 18, 21, 24 or 27 or a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto.

In any embodiment, the polynucleotide (preferably an mRNA) comprises or consists of the nucleic acid sequence set forth in any one of SEQ ID NOs: 2, 10, 11, 13, 16, 19, 22, 25 or 28 or a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto.

In particularly preferred embodiments, the polynucleotide, preferably mRNA, is formulated in a lipid nanoparticle.

The present invention further provides compositions comprising a polynucleotide as described herein, preferably in the form of an mRNA molecule, wherein the composition also comprises a lipid component. The RNA (e.g., mRNA) vaccines of the disclosure can be formulated using one or more liposomes, lipid vesicle, lipoplexes (such as a lipid-polycation complex), or lipid nanoparticles.

In one embodiment, the polynucleotide as described herein is the only polynucleotide present in the composition or in the liposomes, lipid vesicle, lipoplexes (such as a lipid-polycation complex), or lipid nanoparticles. Preferably, the polynucleotide as described herein is the only active ingredient present in the composition or in the liposomes, lipid vesicle, lipoplexes (such as a lipid-polycation complex), or lipid nanoparticles.

In any embodiment, the invention provides a lipid nanoparticle or other nanovehicle, such as nanopolymer, for delivery of the polynucleotide to a subject in need thereof.

Lipid nanoparticles are well known in the art and are further described herein. Preferably the lipid nanoparticle comprises a cationic and/or ionisable lipid, a phospholipid, a PEG (or PEGylated) lipid, and a structural lipid.

In any embodiment, the lipid nanoparticle may comprise
a cationic and/or ionisable lipid comprising from about 40 mol % to about 60 mol % of the total lipid present in the nanoparticle;
a phospholipid comprising from about 5 mol % to about 20 mol % of the total lipid present in the nanoparticle;
a structural lipid comprising from about 30 mol % to about 50 mol % of the total lipid present in the nanoparticle;
a PEGylated lipid comprising from about 0.05 mol % to less than 0.5 mol % of the total lipid present in the nanoparticle.

In any embodiment, the lipid nanoparticle is between about 50-500 nm in diameter. The nanoparticle may have a negative, positive or neutral charge.

In preferred embodiments, the nanoparticle may have a diameter of at least about 100 nm or greater, and have a negative charge.

The present invention also provides a method for producing a lipid nanoparticle comprising a polynucleotide, preferably an mRNA, encoding a chimeric or fusion protein as described herein. Preferably the method comprises formulating any polynucleotide of the invention, with one or more lipids useful for producing a lipid nanoparticle. Preferably the lipid components comprise a phospholipid, a PEG lipid, and a structural lipid.

The present invention also provides a nucleic acid construct or vector, comprising a polynucleotide as described herein.

The vector may be any vector suitable for production of mRNA from a DNA template. The vector may additionally comprise 3′UTR and 5′UTRs and polyadenine fragments.

Examples of such vectors include: IVT mRNA vector or similar vectors that comprise T7, T3 and SP6 signals for expression. The vector can be from a plasmid or produced through PCR or Phi29 DNA polymerase (e.g. GenomiPhi™ V2 DNA) or other bacterial constructs.

The vector may be a self-amplifying RNA vector from alphavirus, such as Venezuelan Equine Encephalitis Virus (VEEV), bipartite VEEV, or variants thereof (including the TC83 mutated variant).

The present invention provides a method for eliciting an immune response to a coronavirus in a subject in need thereof, the method comprising administering to the subject, a polynucleotide, vector, nanoparticle or composition described herein.

The invention provides a method for eliciting an immune response to a coronavirus in a subject in need thereof, the method comprising administering to the subject, a nanoparticle composition comprising:
i) a lipid component; and
ii) an mRNA as described herein, wherein said mRNA is capable of being translated in a cell of the subject to produce the polypeptide encoded by the polynucleotide.

The invention also provides a method for producing an RBD from a coronavirus spike protein in a mammalian cell, the method comprising contacting the mammalian cell with a nanoparticle composition, the composition comprising:
i) a lipid component; and
ii) an mRNA as described herein, wherein said mRNA is capable of being translated in the mammalian cell to produce the RBD.

Preferably the lipid component comprises a cationic and/or ionisable lipid, a phospholipid, a PEG lipid, and a structural lipid.

The invention also provides a method for producing an RBD from a coronavirus spike protein in a mammalian cell, the method comprising contacting the mammalian cell with a nanoparticle composition, the composition comprising:
i) a lipid component; and
ii) an mRNA comprising a polynucleotide sequence encoding a polypeptide,
   wherein the polypeptide comprises: an N-terminal secretion signal peptide; an amino acid sequence of a receptor binding domain (RBD) of a coronavirus spike protein, or variant thereof; and optionally a C-terminal domain comprising a transmembrane region and a cytoplasmic region;
   wherein said mRNA is capable of being translated in the mammalian cell to produce the RBD.

The invention also provides a method for producing an RBD from a SARS-CoV-2 spike protein in a mammalian cell, the method comprising contacting the mammalian cell with a nanoparticle composition, the composition comprising:
i) a lipid component; and
ii) an mRNA comprising a polynucleotide sequence encoding a polypeptide,
   wherein the polypeptide comprises: an N-terminal secretion signal peptide; an amino acid sequence of a receptor binding domain (RBD) of a SARS-CoV-2 spike protein, or variant thereof; and optionally a C-terminal domain comprising a transmembrane region and a cytoplasmic region;
      wherein said mRNA is capable of being translated in the mammalian cell to produce the RBD.

The invention also provides a method for delivering an mRNA to a mammalian cell in a subject in need thereof, said method comprising administering to a subject in need thereof, a nanoparticle composition, the composition comprising:
i) a lipid component; and
ii) an mRNA comprising a polynucleotide sequence as described herein, wherein said mRNA is capable of being translated in the mammalian cell to produce the RBD;
   wherein the administering comprises contacting said mammalian cell with the nanoparticle composition, thereby enabling delivery of the mRNA to the mammalian cell.

Preferably the lipid component comprises a cationic and/or ionisable lipid, a phospholipid, a PEG lipid, and a structural lipid.

The present invention also provides the use of a polynucleotide, vector, or nanoparticle described herein, in the manufacture of a composition for eliciting an immune response to a coronavirus in a subject.

The present invention also provides the use of i) a lipid component as described herein, and ii) a polynucleotide as described herein, in the manufacture of a composition for delivering the polynucleotide to a mammalian cell in a subject in need thereof.

The present invention also provides a polynucleotide, vector, nanoparticle or composition as described herein, for use in eliciting an immune response to a coronavirus in a subject.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Schematic representation of the invention. A) Schematic of coronavirus whole spike protein (derived from Wrapp et al., 2020, Science 367: 1260-1263). The spike protein is a fusion protein comprised of two subunits, S1 and S2. SS=secretion signal; NTD=N-terminal domain; RBD=receptor binding domain from spike protein of a coronavirus; SD1/SD2=subdomains 1 and 2; S2′=S2′ protease cleavage site; FP=fusion peptide; HR1=heptad repeat 1; CH=central helix; CD=connector domain; HR2=heptad repeat 2; TM: transmembrane domain; CT=cytoplasmic tail; B) General design of vaccine antigen; C) Illustration of a polypeptide encoded by an mRNA of the invention and for use in vaccine compositions of the invention.

FIG. 2: Gel analysis of high quality production of long RNA. A) RBD-TM expressed from standard IVT mRNA vector. B) RBD-TM expressed from self-amplifying vector (2 concentrations shown).

FIG. 3: Microscopy images of RBD-TM expression. Green: anti-RBD antibody+AF488 secondary antibody staining. Blue: cell nuclei. A) RBD-TM expressed from standard IVT mRNA vector. B) RBD-TM expressed from self-amplifying vector.

FIG. 4: Inhibition of binding of SARS-CoV-2 spike protein to its receptor (hACE2). RBD-TM design shows superior neutralisation comprised to other vaccine candidates. Starting dilution 1:10. Values represented as mean±SD.

FIG. 5: Microneutralisation assay. Determination of ID ID₅₀, the dilution at which 50% neutralisation of SARS-CoV-2 infection of Vero cells is achieved. The RBD-TM design shows superior neutralisation compared to other candidates. Starting dilution 1:20. Values presented as mean±SD. Dotted line is the limit of detection.

FIG. 6: Sequence optimisation improves level and duration of RBD-TM protein production. Mean fluorescence intensity at 24 hours (A) and 48 hours (B) post-transfection as determined by flow cytometry. A) shows that sequence optimisation improves the level of RBD-TM protein production. B) shows that sequence optimisation improves the duration of RBD-TM protein production.

FIG. 7: Ability of mouse antiserum samples (day 42 after two doses of vaccine) to prevent infection of mammalian cells by two strains of SARS-CoV-2 virus. ID₅₀ values following exposure to two strains of SARS-CoV-2 in mouse model.

Black=ancestral strain; magenta=South African variant B.1.351. An mRNA vaccine of the invention (N/modified) induced potent antibody responses against both strains of SARS-CoV-2 when administered at doses above 1 μg. “30 μg Std” refers to a first-generation product prepared using unmodified mRNA of a full-length S1 protein.

FIG. 8: Development of RBD-neutralising antibodies after prime and boost IM injection of self-amplifying RNA (SAmRNA) in mice. Antibody titres of 10⁴ are indicative of strong activity.

FIG. 9: Day 42 neutralising antibody titres in mice vaccinated intramuscularly with varying doses of WT RBD-TM mRNA vaccine using a prime/boost regime nAB antibody titres against a wild-type (WT) Index strain VIC01 and the Beta B.1.351 variant via micro-neutralisation assay. Titres are expressed as ID50 which is a measure of the extent to which the serum can be diluted but still neutralise the virus. Individual samples from mice (n=5) and mean values are shown, (* p<0.05; no marking=not significant; ANOVA/Tukey's multiple comparison test). The control serum was pooled from previous studies.

FIG. 10: Viral titres in the lungs of BALB/c mice challenged with an N501Y isolate of SARS-CoV-2 Mice vaccinated intramuscularly on days 0 and 21 with 1 μg, 3 μg or 10 μg of WT RBD-TM mRNA vaccine, were aerosol challenged with N501Y (hCoV-19/Australia/VIC2089/2020), 44 days after the second immunisation. Age and sex matched unvaccinated control BALB/c mice were also challenged at the same time. Three days after challenge mice were killed, and the titre of infectious virus (TCID50; 50% tissue culture infectious dose) in the lungs of individual mice were determined by titrating lung supernatants on Vero cell monolayers and measuring viral cytopathic effect (CPE) 5 days later.

FIG. 11: Primary and Secondary total antibody titre responses from mice immunised with RBD-TM (beta variant) vaccine Mice immunised intramuscularly with either 3 μg, 1 μg, 0.3 μg or 0.1 μg of beta variant-derived RBD-TM vaccine in a prime/boost regime on Days 0 and 21. (A) Total antibody titres specific for Beta RBD monomer using Primary sera taken on Day 21 after a single dose of vaccine (B) Total antibody titres specific for WT RBD monomer using Primary sera taken on Day 21 after a single dose of vaccine. (C) Total antibody titres specific for WT RBD monomer using secondary Sera on Day 42. (D) Total antibody titres specific for WT RBD monomer using secondary Sera on Day 56. (* p<0.05; ** p<0.01; *** p<0.001; **** p<0.0001; ANOVA/Tukey's multiple comparison test) (NMS=normal mouse serum, Con=pooled serum from mice treated with 30 μg native mRNA.

FIG. 12: Neutralising antibody titres from mice immunised with Beta RBD-TM vaccine Ability of day 56 serum from mice vaccinated with different doses of Beta RBD-TM to neutralise infection of Vero cells by either “ancestral” (ie wild-type) or Beta variant SARS-CoV-2. ID50 is a measure of the extent to which the serum can be diluted but still neutralise the virus. Dotted line: Limit of detection. Differences between VIC01 and Beta not significant at each dose.

FIG. 13: Neutralisation of a range of surrogate viral RBDs by serum from mice vaccinated with whole spike or RBD-TM vaccines. A surrogate SARS-CoV-2 neutralisation test was used which determines antibody-mediated blockage of ACE2-spike protein-protein interaction. This allows serum to be tested for its ability to block binding of different SARS-CoV-2 variants. The figure shows that vaccines derived from the ancestral (ie wild-type) SARS-CoV-2 strain generate antibodies that bind strongly to the ancestral spike, but also to other variants to different extents. The Omicron variants evade neutralisation to the greatest extent.

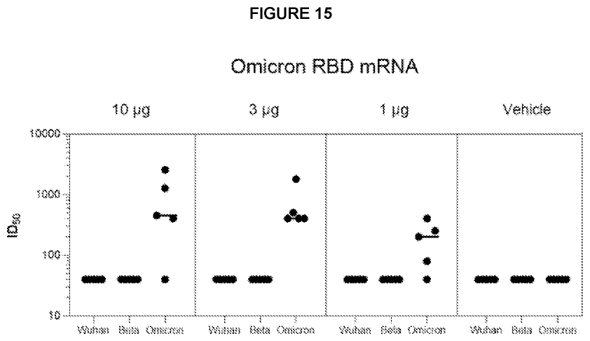

FIG. 14: Neutralisation test on serum from animals vaccinated with Beta RBD-TM. A surrogate SARS-CoV-2 neutralisation test was used which determines antibody-mediated blockage of ACE2-spike protein-protein interaction. This allows serum to be tested for its ability to block binding of different SARS-CoV-2 variants. The figure shows that the Beta vaccine generates antibodies that bind strongly to Beta as well as other variants including the Omicron BA.1 variant.

FIG. 15: Neutralising activity of an Omicron (BA.1) RBD-TM mRNA vaccine against Ancestral, Beta and BA.1 SARS-CoV-2 variants. In this experiment naïve Balb/c mice were vaccinated using a BA.1 variant using the usual regimen of vaccination on days 1 and 21, using doses of either 0, 1, 3 or 10 μg mRNA formulated in an exemplary LNP (as defined in Example 13). Serum collected at day 56 were tested as above using the in vitro microneutralisation test described above to identify whether antibodies induced could protect Vero cells from infection by either the wild type ancestral, Beta or Omicron BA.1 variants of SARS-CoV-2. Doses of 3 or 10 μg BA.1 RBD-TM vaccine induced antibodies that gave very good protection against BA.1 but little or no protection against ancestral or Beta strains of the virus. This illustrates the marked differences between Omicron and the other variants and also demonstrates that the RBD-TM can induce strong specific responses against Omicron.

FIG. 16: Neutralisation test on serum from animals vaccinated with Omicron BA.1 RBD-TM indicates that the BA.1 vaccine has strong activity against BA.1 but weak activity against the ancestral SARS-CoV-2 and other variants. A surrogate SARS-CoV-2 neutralisation test was used which determines antibody-mediated blockage of ACE2-spike protein-protein interaction. This allows serum to be tested for its ability to block binding of different SARS-CoV-2 variants. The figure shows that the Omicron BA.1 vaccine generates antibodies that bind strongly to Omicron BA.1 but these have limited activity against other variants.

FIG. 17: Total Antibody responses and neutralising antibody responses 21 and 42 days after IM vaccination of mice with either WT whole spike or WT RBD-TM vaccines using an LNP formulation at 1 or 5 μg doses All mice were vaccinated intramuscularly with either whole spike protein mRNA vaccine or RBD-TM mRNA vaccine in an LNP formulation at doses if 1 μg or 5 μg in a prime boost regime on days 0 and 21. Sera collected on day 42 (21 days after the secondary immunisation) was used to assess antibody responses. (A) Total antibody titres specific for WT RBD monomer 21 days post vaccination (B) Total antibody titres specific for WT RBD monomer 42 days post vaccination (NMS=normal mouse serum, Con=pooled serum from mice treated with 30 μg native mRNA. (** p<0.01; *** p<0.001. ; ANOVA/Tukey's multiple comparison test) (C) Neutralising antibody response using the mNT assay. Levels of nABs able to neutralise infection in Vero cells of either WT (VIC01) or Beta SARS-CoV-2 variants. ID₅₀ is a measure of the extent to which the serum can be diluted but still neutralise the virus.

FIG. 18: Total Antibody responses and neutralising antibody responses 21 and 42 days after IM vaccination of mice with either WT whole spike or WT RBD-TM vaccines with the alternative LNP formulation based on Comirnaty™ at 1 or 5 μg doses. All mice were vaccinated intramuscularly with either whole spike protein mRNA vaccine or RBD-TM mRNA vaccine in the alternative LNP formulation based on the Comirnaty™ vaccine at doses if 1 μg or 5 μg in a prime boost regime on days 0 and 21. Sera collected on day 21 and 42 (0 and 21 days after the secondary immunisation) were used to assess antibody responses. (A) Total antibody titres specific for WT RBD monomer 21 days post vaccination (B) Total antibody titres specific for WT RBD monomer 42 days post vaccination (NMS=normal mouse serum, Con=pooled serum from mice treated with 30 μg native mRNA (FIG. 17) (*** p<0.001; ANOVA/Tukey's multiple comparison test; no marking=not significant) (C) Neutralising antibody response using the mNT assay. Levels of nABs able to neutralise infection in Vero cells of either WT (VIC01) or Beta SARS-CoV-2 variants. ID50 is a measure of the extent to which the serum can be diluted but still neutralise the virus. Dotted lined=limit of detection.

FIG. 19: Wild-type and Beta specific total and neutralising antibody responses after prime and boost vaccination with 3 μg Beta RBD-TM delivered using different Lipids and LNP formulations combinations. 5 BALB/c mice were vaccinated with 3 μg doses of Beta RBD-TM mRNA on days 1 and 21 with the four different LNP formulations based on the specified lipids and lipid ratios. (A) Total antibody titres against wild-type RBD using sera collected at day 21 (B) Total antibody titres against Beta RBD using sera collected on Day 21. (C) Total antibody titres against wild-type RBD using sera collected at day 42 (D) Total antibody titres against Beta RBD using sera collected on Day 42. (NMS=normal mouse serum, Con=pooled serum from mice treated with 30 μg native mRNA (FIG. 17). (E) Neutralisation of VIC01 and Beta strains of virus using sera collected at Day 42. In this experiment the NIBSC control (which should have an ID50 of 1000) had and ID50 of 504. The raw data is shown in panel E but based on the NIBSC control the mean ID50 values can be normalised by multiplying by a factor of 2, which brings the data in line with that shown in FIGS. 12 and 13. (* p<0.05; *** p<0.001; ANOVA/Tukey's multiple comparison test; no marking=not significant). (VIC01 refers to an isolate of wildtype (WT) RBD-TM mRNA and Beta refers to beta variant RBD-TM. Acuitas Std refers to formulation 1; Acuitas MIPS refers to formulation 2; MIPS std refers to formulation 3 and MIPS MIPS refers to formulation 4, respectively in Example 13; comparison 3).

FIG. 20: Viral titres in lung homogenates and nasal turbinates three days after challenge with Beta SARS-CoV-2. Mice received prime and boost vaccination with 3 μg Beta RBD-TM on days 1 and 21, delivered using the four different formulations described in Example 13.

### Sequence Information

TABLE 1
Sequence information

| | SEQ ID | |
|---|---|---|
| Description | NO: | Sequence |
| Full length | 1 | MFVFLVLLPLVSSQCVNLTTRTQLPPAYTNSFTRGVY |
| polypeptide | | YPDKVFRSSVLHSTQDLFLPFFSNVTWFHAIHVSGTN |
| sequence of | | GTKRFDNPVLPFNDGVYFASTEKSNIIRGWIFGTTLD |
| SARS-COV-2 | | SKTQSLLIVNNATNVVIKVCEFQFCNDPFLGVYYHKN |
| spike protein | | NKSWMESEFRVYSSANNCTFEYVSQPFLMDLEGKQGN |
| (RBD shown | | FKNLREFVFKNIDGYFKIYSKHTPINLVRDLPQGFSA |
| underlined; | | LEPLVDLPIGINITRFQTLLALHRSYLTPGDSSSGWT |
| signal peptide | | AGAAAYYVGYLQPRTFLLKYNENGTITDAVDCALDPL |
| italicised; | | SETKCTLKSFTVEKGIYQTSNFRVQPTESIVRFPNIT |
| transmembrane | | NLCPFGEVFNATRFASVYAWNRKRISNCVADYSVLYN |
| domain + | | SASFSTFKCYGVSPTKLNDLCFTNVYADSFVIRGDEV |
| cytoplasmic | | RQIAPGQTGKIADYNYKLPDDFTGCVIAWNSNNLDSK |
| domain in | | VGGNYNYLYRLFRKSNLKPFERDISTEIYQAGSTPCN |
| bold) | | GVEGENCYFPLQSYGFQPTNGVGYQPYRVVVLSFELL |
| | | HAPATVCGPKKSTNLVKNKCVNFNFNGLTGTGVLTES |
| | | NKKFLPFQQFGRDIADTTDAVRDPQTLEILDITPCSF |
| | | GGVSVITPGTNTSNQVAVLYQDVNCTEVPVAIHADQL |
| | | TPTWRVYSTGSNVFQTRAGCLIGAEHVNNSYECDIPI |
| | | GAGICASYQTQTNSPRRARSVASQSIIAYTMSLGAEN |
| | | SVAYSNNSIAIPTNFTISVTTEILPVSMTKTSVDCTM |
| | | YICGDSTECSNLLLQYGSFCTQLNRALTGIAVEQDKN |
| | | TQEVFAQVKQIYKTPPIKDFGGFNFSQILPDPSKPSK |
| | | RSFIEDLLFNKVTLADAGFIKQYGDCLGDIAARDLIC |
| | | AQKFNGLTVLPPLLTDEMIAQYTSALLAGTITSGWTF |
| | | GAGAALQIPFAMQMAYRFNGIGVTQNVLYENQKLIAN |
| | | QFNSAIGKIQDSLSSTASALGKLQDVVNQNAQALNTL |
| | | VKQLSSNFGAISSVLNDILSRLDKVEAEVQIDRLITG |
| | | RLQSLQTYVTQQLIRAAEIRASANLAATKMSECVLGQ |
| | | SKRVDFCGKGYHLMSFPQSAPHGVVFLHVTYVPAQEK |
| | | NFTTAPAICHDGKAHFPREGVFVSNGTHWFVTQRNFY |
| | | EPQIITTDNTFVSGNCDVVIGIVNNTVYDPLQPELDS |
| | | FKEELDKYFKNHTSPDVDLGDISGINASVVNIQKEID |
| | | RLNEVAKNLNESLIDLQELGKYEQYIKWPWYIWLGFI |
| | | AGLIAIVMVTIMLCCMTSCCSCLKGCCSCGSCCKFDE |
| | | DDSEPVLKGVKLHYT |
| Exemplary | 2 | AUGUUUgUUUUUcUUgUUUUaUUgccacUagUcUcUa |
| polynucleotide | | gUcagUgUgUUaaUcUUacaaccggaaUcUaUcaaac |
| sequence of the | | UUcUaacUUUagagUccaaccaacagaaUcUaUUgUU |
| invention | | agaUUUccUaaUaUUacaaacUUgUgcccUUUUggUg |
| | | aagUUUUUaacgccaccagaUUUgcaUcUgUUUaUgc |
| | | UUggaacaggaagagaaUcagcaacUgUgUUgcUgaU |
| | | UaUUcUgUccUaUaUaaUUccgcaUcaUUUUccacUU |
| | | UUaagUgUUaUggagUgUcUccUacUaaaUUaaaUga |
| | | UcUcUgcUUUacUaaUgUcUaUgcagaUUcaUUUgUa |
| | | aUUagaggUgaUgaagUcagacaaaUcgcUccagggc |
| | | aaacUggaaagaUUgcUgaUUaUaaUUaUaaaUUacc |
| | | agaUgaUUUUacaggcUgcgUUaUagcUUggaaUUcU |
| | | aacaaUcUUgaUUcUaaggUUggUggUaaUUaUaaUU |
| | | accUgUaUagaUUgUUUaggaagUcUaaUcUcaaacc |
| | | UUUUgagagagaUaUUUcaacUgaaaUcUaUcaggcc |
| | | ggUagcacaccUUgUaaUggUgUUgaaggUUUUaaUU |
| | | gUUacUUUccUUUacaaUcaUaUggUUUccaacccac |
| | | UaaUggUgUUggUUaccaaccaUacagagUagUagUa |
| | | cUUUcUUUUgaacUUcUacaUgcaccagcaacUgUUU |
| | | gUggaccUaaaaagUcUacUaaUUUggUUaaaaacaa |
| | | aUgUgUcaaUUUcaacUUcaaUggUGGUUCCUCCGGU |
| | | GGUUaUgagcagUaUaUaaaaUggccaUggUacaUUU |
| | | ggcUaggUUUUaUagcUggcUUgaUUgccaUagUaaU |
| | | ggUgacaaUUaUgcUUUgcUgUaUgaccagUUgcUgU |
| | | agUUgUcUcaagggcUgUUgUUcUUgUggaUccUgcU |
| | | gcaaaUUUgaUgaagacgacUcUgagccagUgcUcaa |
| | | aggagUcaaaUUacaUUacacaUAAUGA |
| Exemplary | 3 | MFVFLVLLPLVSSQCVNLTTGIYQTSNFRVQPTESIV |
| polypeptide | | RFPNITNLCPFGEVFNATRFASVYAWNRKRISNCVAD |
| encoded by a | | YSVLYNSASFSTFKCYGVSPTKLNDLCFTNVYADSFV |
| polynucleotide of | | IRGDEVRQIAPGQTGKIADYNYKLPDDFTGCVIAWNS |
| the invention. N | | NNLDSKVGGNYNYLYRLFRKSNLKPFERDISTEIYQA |
| terminal signal | | GSTPCNGVEGFNCYFPLQSYGFQPTNGVGYQPYRVVV |
| peptide | | LSFELLHAPATVCGPKKSTNLVKNKCVNFNFNGGSSG |
| underlined; linker | | GYEQYIKWPWYIWLGFIAGLIAIVMVTIMLCCMTSCC |
| sequence in bold; | | SCLKGCCSCGSCCKFDEDDSEPVLKGVKLHYT |
| C-terminal domain | | |
| comprising | | |
| transmembrane | | |
| and cytoplasmic | | |
| region in italics | | |
| SARS COV-2 | 4 | MFVFLVLLPLVSSQCVNLTT |
| spike protein N- | | |
| terminal secretion | | |
| signal peptide | | |
| Exemplary | 5 | WPWYIWLGFIAGLIAIVMVTIMLCCMTSCCSCLK |
| transmembrane | | |
| domain of SARS- | | |
| Co-V2 spike | | |
| protein | | |
| Exemplary | 6 | YEQWPWYIWLGFIAGLIAIVMVTIML |
| transmembrane | | |
| domain of SARS- | | |
| Co-V2 spike | | |
| protein | | |
| Exemplary | 7 | GCCSCGSCCKFDEDDSEPVLKGVKLHYT |
| cytoplasmic region | | |
| of SARS-Co-V2 | | |
| spike protein | | |
| Exemplary | 8 | YEQYIKWPWYIWLGFIAGLIAIVMVTIMLCCMTSCCS |
| transmembrane | | CLKGCCSCGSCCKFDEDDSEPVLKGVKLHYT |
| domain and | | |
| cytoplasmic region | | |
| of SARS-Co-V2 | | |
| spike protein | | |
| Exemplary SARS- | 9 | GIYQTSNFRVQPTESIVRFPNITNLCPFGEVFNATRF |
| CoV-2 RBD | | ASVYAWNRKRISNCVADYSVLYNSASFSTFKCYGVSP |
| | | TKLNDLCFTNVYADSFVIRGDEVRQIAPGQTGKIADY |
| | | NYKLPDDFTGCVIAWNSNNLDSKVGGNYNYLYRLFRK |
| | | SNLKPFERDISTEIYQAGSTPCNGVEGFNCYFPLQSY |
| | | GFQPTNGVGYQPYRVVVLSFELLHAPATVCGPKKSTN |
| | | LVKNKCVNFNFN |
| Optimised RBD- | 10 | AUGUUCGUGUUCCUGGUGCUGCUGCCCCUGGUGAGCA |
| TM RNA | | GCCAGUGCGUGAACCUGACCACCGGCAUCUACCAGAC |
| sequence (codon | | CAGCAACUUCAGGGUGCAGCCCACCGAGAGCAUCGUG |
| depletion of | | AGGUUCCCCAACAUCACCAACCUGUGCCCCUUCGGCG |
| uridine) | | AGGUGUUCAACGCCACCAGGUUCGCGAGCGUGUACGC |
| | | CUGGAACAGGAAGAGGAUCAGCAACUGCGUGGCCGAC |
| | | UACAGCGUGCUGUACAACAGCGCCAGCUUCAGCACCU |
| | | UCAAGUGCUACGGCGUGAGCCCCACCAAGUUAAACGA |
| | | CCUGUGCUUCACCAACGUGUACGCCGACAGCUUCGUG |
| | | AUCAGGGGCGACGAGGUGAGGCAGAUCGCCCCCGGCC |
| | | AGACCGGCAAGAUCGCCGACUACAACUACAAGCUGCC |
| | | CGACGACUUCACCGGCUGCGUGAUCGCCUGGAACAGC |
| | | AACAACCUGGACAGCAAGGUGGGCGGCAACUACAACU |
| | | ACCUGUACAGGCUGUUCAGGAAGAGCAACCUGAAGCC |
| | | CUUCGAGAGGGACAUCAGCACCGAGAUCUACCAGGCC |
| | | GGCAGCACCCCCUGCAACGGCGUGGAGGGCUUCAACU |
| | | GCUACUUCCCCCUGCAGAGCUACGGCUUCCAGCCCAC |
| | | CAACGGCGUGGGCUACCAGCCCUACAGAGUGGUGGUG |
| | | CUGAGCUUCGAGCUGCUGCACGCCCCCGCCACCGUGU |
| | | GCGGCCCCAAGAAGAGCACGAACCUCGUGAAGAACAA |
| | | GUGCGUGAACUUCAACUUCAACGGCGGCAGCUCCGGC |
| | | GGCUACGAGCAGUACAUCAAGUGGCCCUGGUACAUCU |
| | | GGCUGGGCUUCAUCGCCGGCCUGAUCGCCAUCGUGAU |
| | | GGUGACCAUCAUGCUGUGCUGCAUGACCAGCUGCUGC |
| | | AGCUGCCUGAAGGGCUGCUGCAGCUGCGGCAGCUGCU |
| | | GCAAGUUCGACGAGGACGACAGCGAGCCCGUGCUGAA |
| | | GGGCGUGAAGCUGCACUACACCUAAUGA |
| Optimised RBD- | 11 | AUGUUCGUGUUCCUGGUGCUGCUGCCCCUGGUGUCGU |
| TM RNA | | CGCAGUGCGUGAACCUGACCACCGGCAUCUACCAGAC |
| sequence | | CUCGAACUUCAGGGUGCAGCCCACCGAGUCGAUCGUG |
| comprising codon | | AGGUUCCCCAACAUCACCAACCUGUGCCCCUUCGGCG |
| depletion of | | AGGUGUUCAACGCCACCAGGUUCGCGUCGGUGUACGC |
| uridines and | | CUGGAACAGGAAGAGGAUCAGCAACUGCGUGGCCGAC |
| where codons | | UACUCGGUGCUGUACAACUCGGCCUCGUUCUCGACCU |
| encoding serine | | UCAAGUGCUACGGCGUGUCGCCCACCAAGUUAAACGA |
| amino acids are | | CCUGUGCUUCACCAACGUGUACGCCGACUCGUUCGUG |
| converted to UCG | | AUCAGGGGCGACGAGGUGAGGCAGAUCGCCCCCGGCC |
| | | AGACCGGCAAGAUCGCCGACUACAACUACAAGCUGCC |
| | | CGACGACUUCACCGGCUGCGUGAUCGCCUGGAACUCG |
| | | AACAACCUGGACAGCAAGGUGGGCGGCAACUACAACU |
| | | ACCUGUACAGGCUGUUCAGGAAGUCGAACCUGAAGCC |
| | | CUUCGAGAGGGACAUCUCGACCGAGAUCUACCAGGCC |
| | | GGCUCGACCCCCUGCAACGGCGUGGAGGGCUUCAACU |
| | | GCUACUUCCCCCUGCAGUCGUACGGCUUCCAGCCCAC |
| | | CAACGGCGUGGGCUACCAGCCCUACAGAGUGGUGGUG |
| | | CUGUCGUUCGAGCUGCUGCACGCCCCCGCCACCGUGU |
| | | GCGGCCCCAAGAAGUCGACGAACCUCGUGAAGAACAA |
| | | GUGCGUGAACUUCAACUUCAACGGCGGCUCGUCGGGC |
| | | GGCUACGAGCAGUACAUCAAGUGGCCCUGGUACAUCU |
| | | GGCUGGGCUUCAUCGCCGGCCUGAUCGCCAUCGUGAU |
| | | GGUGACCAUCAUGCUGUGCUGCAUGACCUCGUGCUGC |
| | | UCGUGCCUGAAGGGCUGCUGCUCGUGCGGCUCGUGCU |
| | | GCAAGUUCGACGAGGACGACUCGGAGCCCGUGCUGAA |
| | | GGGCGUGAAGCUGCACUACACCUAAUGA |
| RBD-TM | 12 | MFVFLVLLPLVSSQCVNLTTGIYQTSNFRVQPTESIV |
| ancestral “wild- | | RFPNITNLCPFGEVFNATRFASVYAWNRKRISNCVAD |
| type” | | YSVLYNSASFSTFKCYGVSPTKLNDLCFTNVYADSFV |
| Amino acid | | IRGDEVRQIAPGQTGKIADYNYKLPDDFTGCVIAWNS |
| sequence | | NNLDSKVGGNYNYLYRLFRKSNLKPFERDISTEIYQA |
| Signal peptide | | GSTPCNGVEGFNCYFPLQSYGFQPTNGVGYQPYRVVV |
| (underline) | | LSFELLHAPATVCGPKKSTNLVKNKCVNFNFNGGSSG |
| Linker (bold) | | GYEQYIKWPWYIWLGFIAGLIAIVMVTIMLCCMTSCC |
| C-terminal domain | | SCLKGCCSCGSCCKFDEDDSEPVLKGVKLHYT |
| comprising | | |
| transmembrane | | |
| and cytoplasmic | | |
| region | | |
| RBD-TM | 13 | AUGUUCGUGUUCCUGGUGCUGCUGCCCCUGGUGAGCA |
| ancestral “wild- | | GCCAGUGCGUGAACCUGACCACCGGCAUCUACCAGAC |
| type” | | CAGCAACUUCAGGGUGCAGCCCACCGAGAGCAUCGUG |
| mRNA sequence | | AGGUUCCCCAACAUCACCAACCUGUGCCCCUUCGGCG |
| Signal peptide | | AGGUGUUCAACGCCACCAGGUUCGCGAGCGUGUACGC |
| (underline) | | CUGGAACAGGAAGAGGAUCAGCAACUGCGUGGCCGAC |
| Linker (bold) | | UACAGCGUGCUGUACAACAGCGCCAGCUUCAGCACCU |
| C-terminal domain | | UCAAGUGCUACGGCGUGAGCCCCACCAAGUUAAACGA |
| comprising | | CCUGUGCUUCACCAACGUGUACGCCGACAGCUUCGUG |
| transmembrane | | AUCAGGGGCGACGAGGUGAGGCAGAUCGCCCCCGGCC |
| and cytoplasmic | | AGACCGGCAAGAUCGCCGACUACAACUACAAGCUGCC |
| region | | CGACGACUUCACCGGCUGCGUGAUCGCCUGGAACAGC |
| Stop codons | | AACAACCUGGACAGCAAGGUGGGCGGCAACUACAACU |
| | | ACCUGUACAGGCUGUUCAGGAAGAGCAACCUGAAGCC |
| | | CUUCGAGAGGGACAUCAGCACCGAGAUCUACCAGGCC |
| | | GGCAGCACCCCCUGCAACGGCGUGGAGGGCUUCAACU |
| | | GCUACUUCCCCCUGCAGAGCUACGGCUUCCAGCCCAC |
| | | CAACGGCGUGGGCUACCAGCCCUACAGAGUGGUGGUG |
| | | CUGAGCUUCGAGCUGCUGCACGCCCCCGCCACCGUGU |
| | | GCGGCCCCAAGAAGAGCACGAACCUCGUGAAGAACAA |
| | | GUGCGUGAACUUCAACUUCAACGGCGGCAGCUCCGGC |
| | | GGCUACGAGCAGUACAUCAAGUGGCCCUGGUACAUCU |
| | | GGCUGGGCUUCAUCGCCGGCCUGAUCGCCAUCGUGAU |
| | | GGUGACCAUCAUGCUGUGCUGCAUGACCAGCUGCUGC |
| | | AGCUGCCUGAAGGGCUGCUGCAGCUGCGGCAGCUGCU |
| | | GCAAGUUCGACGAGGACGACAGCGAGCCCGUGCUGAA |
| | | GGGCGUGAAGCUGCACUACACCUAAUGA |
| RBD-TM | 14 | ATGTTCGTGTTCCTGGTGCTGCTGCCCCTGGTGAGCA |
| ancestral “wild- | | GCCAGTGCGTGAACCTGACCACCGGCATCTACCAGAC |
| type” | | CAGCAACTTCAGGGTGCAGCCCACCGAGAGCATCGTG |
| DNA sequence | | AGGTTCCCCAACATCACCAACCTGTGCCCCTTCGGCG |
| Signal peptide | | AGGTGTTCAACGCCACCAGGTTCGCGAGCGTGTACGC |
| (underline) | | CTGGAACAGGAAGAGGATCAGCAACTGCGTGGCCGAC |
| Linker (bold) | | TACAGCGTGCTGTACAACAGCGCCAGCTTCAGCACCT |
| C-terminal domain | | TCAAGTGCTACGGCGTGAGCCCCACCAAGTTAAACGA |
| comprising | | CCTGTGCTTCACCAACGTGTACGCCGACAGCTTCGTG |
| transmembrane | | ATCAGGGGCGACGAGGTGAGGCAGATCGCCCCCGGCC |
| and cytoplasmic | | AGACCGGCAAGATCGCCGACTACAACTACAAGCTGCC |
| region | | CGACGACTTCACCGGCTGCGTGATCGCCTGGAACAGC |
| Stop codons | | AACAACCTGGACAGCAAGGTGGGCGGCAACTACAACT |
| | | ACCTGTACAGGCTGTTCAGGAAGAGCAACCTGAAGCC |
| | | CTTCGAGAGGGACATCAGCACCGAGATCTACCAGGCC |
| | | GGCAGCACCCCCTGCAACGGCGTGGAGGGCTTCAACT |
| | | GCTACTTCCCCCTGCAGAGCTACGGCTTCCAGCCCAC |
| | | CAACGGCGTGGGCTACCAGCCCTACAGAGTGGTGGTG |
| | | CTGAGCTTCGAGCTGCTGCACGCCCCCGCCACCGTGT |
| | | GCGGCCCCAAGAAGAGCACGAACCTCGTGAAGAACAA |
| | | GTGCGTGAACTTCAACTTCAACGGCGGCAGCTCCGGC |
| | | GGCTACGAGCAGTACATCAAGTGGCCCTGGTACATCT |
| | | GGCTGGGCTTCATCGCCGGCCTGATCGCCATCGTGAT |
| | | GGTGACCATCATGCTGTGCTGCATGACCAGCTGCTGC |
| | | AGCTGCCTGAAGGGCTGCTGCAGCTGCGGCAGCTGCT |
| | | GCAAGTTCGACGAGGACGACAGCGAGCCCGTGCTGAA |
| | | GGGCGTGAAGCTGCACTACACCTAATGA |
| RBD-TM Beta | 15 | MFVFLVLLPLVSSQCVNLTTGIYQTSNFRVQPTESIV |
| variant | | RFPNITNLCPFGEVFNATRFASVYAWNRKRISNCVAD |
| Amino acid | | YSVLYNSASFSTFKCYGVSPTKLNDLCFTNVYADSFV |
| sequence | | IRGDEVRQIAPGQTGNIADYNYKLPDDFTGCVIAWNS |
| Signal peptide | | NNLDSKVGGNYNYLYRLFRKSNLKPFERDISTEIYQA |
| (underline) | | GSTPCNGVKGFNCYFPLQSYGFQPTYGVGYQPYRVVV |
| Linker (bold) | | LSFELLHAPATVCGPKKSTNLVKNKCVNFNFNGGSSG |
| Mutations K417N, | | GYEQYIKWPWYIWLGFIAGLIAIVMVTIMLCCMTSCC |
| E484K & N501Y | | SCLKGCCSCGSCCKFDEDDSEPVLKGVKLHYT |
| C-terminal domain | | |
| comprising | | |
| transmembrane | | |
| and cytoplasmic | | |
| region | | |
| Stop codons | | |
| RBD-TM Beta | 16 | AUGUUCGUGUUCCUGGUGCUGCUGCCCCUGGUGAGCA |
| variant | | GCCAGUGCGUGAACCUGACCACCGGCAUCUACCAGAC |
| mRNA sequence | | CAGCAACUUCAGGGUGCAGCCCACCGAGAGCAUCGUG |
| Signal peptide | | AGGUUCCCCAACAUCACCAACCUGUGCCCCUUCGGCG |
| (underline) | | AGGUGUUCAACGCCACCAGGUUCGCGAGCGUGUACGC |
| Linker (bold) | | CUGGAACAGGAAGAGGAUCAGCAACUGCGUGGCCGAC |
| Mutations K417N, | | UACAGCGUGCUGUACAACAGCGCCAGCUUCAGCACCU |
| E484K & N501Y | | UCAAGUGCUACGGCGUGAGCCCCACCAAGUUAAACGA |
| C-terminal domain | | CCUGUGCUUCACCAACGUGUACGCCGACAGCUUCGUG |
| comprising | | AUCAGGGGCGACGAGGUGAGGCAGAUCGCCCCCGGCC |
| transmembrane | | AGACCGGCAACAUCGCCGACUACAACUACAAGCUGCC |
| and cytoplasmic | | CGACGACUUCACCGGCUGCGUGAUCGCCUGGAACAGC |
| region | | AACAACCUGGACAGCAAGGUGGGCGGCAACUACAACU |
| Stop codons | | ACCUGUACAGGCUGUUCAGGAAGAGCAACCUGAAGCC |
| | | CUUCGAGAGGGACAUCAGCACCGAGAUCUACCAGGCC |
| | | GGCAGCACCCCCUGCAACGGCGUGAAGGGCUUCAACU |
| | | GCUACUUCCCCCUGCAGAGCUACGGCUUCCAGCCCAC |
| | | CUACGGCGUGGGCUACCAGCCCUACAGAGUGGUGGUG |
| | | CUGAGCUUCGAGCUGCUGCACGCCCCCGCCACCGUGU |
| | | GCGGCCCCAAGAAGAGCACGAACCUCGUGAAGAACAA |
| | | GUGCGUGAACUUCAACUUCAACGGCGGCAGCUCCGGC |
| | | GGCUACGAGCAGUACAUCAAGUGGCCCUGGUACAUCU |
| | | GGCUGGGCUUCAUCGCCGGCCUGAUCGCCAUCGUGAU |
| | | GGUGACCAUCAUGCUGUGCUGCAUGACCAGCUGCUGC |
| | | AGCUGCCUGAAGGGCUGCUGCAGCUGCGGCAGCUGCU |
| | | GCAAGUUCGACGAGGACGACAGCGAGCCCGUGCUGAA |
| | | GGGCGUGAAGCUGCACUACACCUAAUGA |
| RBD-TM Beta | 17 | ATGTTCGTGTTCCTGGTGCTGCTGCCCCTGGTGAGCA |
| variant | | GCCAGTGCGTGAACCTGACCACCGGCATCTACCAGAC |
| DNA sequence | | CAGCAACTTCAGGGTGCAGCCCACCGAGAGCATCGTG |
| Signal peptide | | AGGTTCCCCAACATCACCAACCTGTGCCCCTTCGGCG |
| Mutations K417N, | | AGGTGTTCAACGCCACCAGGTTCGCGAGCGTGTACGC |
| E484K & N501Y | | CTGGAACAGGAAGAGGATCAGCAACTGCGTGGCCGAC |
| Linker | | TACAGCGTGCTGTACAACAGCGCCAGCTTCAGCACCT |
| C-terminal domain | | TCAAGTGCTACGGCGTGAGCCCCACCAAGTTAAACGA |
| comprising | | CCTGTGCTTCACCAACGTGTACGCCGACAGCTTCGTG |
| transmembrane | | ATCAGGGGCGACGAGGTGAGGCAGATCGCCCCCGGCC |
| and cytoplasmic | | AGACCGGCAACATCGCCGACTACAACTACAAGCTGCC |
| region | | CGACGACTTCACCGGCTGCGTGATCGCCTGGAACAGC |
| Stop codons | | AACAACCTGGACAGCAAGGTGGGCGGCAACTACAACT |
| | | ACCTGTACAGGCTGTTCAGGAAGAGCAACCTGAAGCC |
| | | CTTCGAGAGGGACATCAGCACCGAGATCTACCAGGCC |
| | | GGCAGCACCCCCTGCAACGGCGTGAAGGGCTTCAACT |
| | | GCTACTTCCCCCTGCAGAGCTACGGCTTCCAGCCCAC |
| | | CTACGGCGTGGGCTACCAGCCCTACAGAGTGGTGGTG |
| | | CTGAGCTTCGAGCTGCTGCACGCCCCCGCCACCGTGT |
| | | GCGGCCCCAAGAAGAGCACGAACCTCGTGAAGAACAA |
| | | GTGCGTGAACTTCAACTTCAACGGCGGCAGCTCCGGC |
| | | GGCTACGAGCAGTACATCAAGTGGCCCTGGTACATCT |
| | | GGCTGGGCTTCATCGCCGGCCTGATCGCCATCGTGAT |
| | | GGTGACCATCATGCTGTGCTGCATGACCAGCTGCTGC |
| | | AGCTGCCTGAAGGGCTGCTGCAGCTGCGGCAGCTGCT |
| | | GCAAGTTCGACGAGGACGACAGCGAGCCCGTGCTGAA |
| | | GGGCGTGAAGCTGCACTACACCTAATGA |
| RBD-TM Delta | 18 | MFVFLVLLPLVSSQCVNLTTGIYQTSNFRVQPTESIV |
| variant | | RFPNITNLCPFGEVFNATRFASVYAWNRKRISNCVAD |
| Amino acid | | YSVLYNSASFSTFKCYGVSPTKLNDLCFTNVYADSFV |
| sequence | | IRGDEVRQIAPGQTGKIADYNYKLPDDFTGCVIAWNS |
| Signal peptide | | NNLDSKVGGNYNYRYRLFRKSNLKPFERDISTEIYQA |
| Mutations | | GSKPCNGVEGFNCYFPLQSYGFQPTNGVGYQPYRVVV |
| L452R & T478K | | LSFELLHAPATVCGPKKSTNLVKNKCVNFNFNGGSSG |
| Linker | | GYEQYIKWPWYIWLGFIAGLIAIVMVTIMLCCMTSCC |
| C-terminal domain | | SCLKGCCSCGSCCKFDEDDSEPVLKGVKLHYT |
| comprising | | |
| transmembrane | | |
| and cytoplasmic | | |
| region | | |
| Stop codons | | |
| RBD-TM Delta | 19 | AUGUUCGUGUUCCUGGUGCUGCUGCCCCUGGUGAGCA |
| variant | | GCCAGUGCGUGAACCUGACCACCGGCAUCUACCAGAC |
| mRNA sequence | | CAGCAACUUCAGGGUGCAGCCCACCGAGAGCAUCGUG |
| Signal peptide | | AGGUUCCCCAACAUCACCAACCUGUGCCCCUUCGGCG |
| Mutations L452R | | AGGUGUUCAACGCCACCAGGUUCGCGAGCGUGUACGC |
| & T478K | | CUGGAACAGGAAGAGGAUCAGCAACUGCGUGGCCGAC |
| Linker | | UACAGCGUGCUGUACAACAGCGCCAGCUUCAGCACCU |
| C-terminal domain | | UCAAGUGCUACGGCGUGAGCCCCACCAAGUUAAACGA |
| comprising | | CCUGUGCUUCACCAACGUGUACGCCGACAGCUUCGUG |
| transmembrane | | AUCAGGGGCGACGAGGUGAGGCAGAUCGCCCCCGGCC |
| and cytoplasmic | | AGACCGGCAAGAUCGCCGACUACAACUACAAGCUGCC |
| region | | CGACGACUUCACCGGCUGCGUGAUCGCCUGGAACAGC |
| Stop codons | | AACAACCUGGACAGCAAGGUGGGCGGCAACUACAACU |
| | | ACAGGUACAGGCUGUUCAGGAAGAGCAACCUGAAGCC |
| | | CUUCGAGAGGGACAUCAGCACCGAGAUCUACCAGGCC |
| | | GGCAGCAAGCCCUGCAACGGCGUGGAGGGCUUCAACU |
| | | GCUACUUCCCCCUGCAGAGCUACGGCUUCCAGCCCAC |
| | | CAACGGCGUGGGCUACCAGCCCUACAGAGUGGUGGUG |
| | | CUGAGCUUCGAGCUGCUGCACGCCCCCGCCACCGUGU |
| | | GCGGCCCCAAGAAGAGCACGAACCUCGUGAAGAACAA |
| | | GUGCGUGAACUUCAACUUCAACGGCGGCAGCUCCGGC |
| | | GGCUACGAGCAGUACAUCAAGUGGCCCUGGUACAUCU |
| | | GGCUGGGCUUCAUCGCCGGCCUGAUCGCCAUCGUGAU |
| | | GGUGACCAUCAUGCUGUGCUGCAUGACCAGCUGCUGC |
| | | AGCUGCCUGAAGGGCUGCUGCAGCUGCGGCAGCUGCU |
| | | GCAAGUUCGACGAGGACGACAGCGAGCCCGUGCUGAA |
| | | GGGCGUGAAGCUGCACUACACCUAAUGA |
| RBD-TM Delta | 20 | ATGTTCGTGTTCCTGGTGCTGCTGCCCCTGGTGAGCA |
| variant | | GCCAGTGCGTGAACCTGACCACCGGCATCTACCAGAC |
| DNA sequence | | CAGCAACTTCAGGGTGCAGCCCACCGAGAGCATCGTG |
| Signal peptide | | AGGTTCCCCAACATCACCAACCTGTGCCCCTTCGGCG |
| Mutations | | AGGTGTTCAACGCCACCAGGTTCGCGAGCGTGTACGC |
| L452R & T478K | | CTGGAACAGGAAGAGGATCAGCAACTGCGTGGCCGAC |
| Linker | | TACAGCGTGCTGTACAACAGCGCCAGCTTCAGCACCT |
| C-terminal domain | | TCAAGTGCTACGGCGTGAGCCCCACCAAGTTAAACGA |
| comprising | | CCTGTGCTTCACCAACGTGTACGCCGACAGCTTCGTG |
| transmembrane | | ATCAGGGGCGACGAGGTGAGGCAGATCGCCCCCGGCC |
| and cytoplasmic | | AGACCGGCAAGATCGCCGACTACAACTACAAGCTGCC |
| region | | CGACGACTTCACCGGCTGCGTGATCGCCTGGAACAGC |
| Stop codons | | AACAACCTGGACAGCAAGGTGGGCGGCAACTACAACT |
| | | ACAGGTACAGGCTGTTCAGGAAGAGCAACCTGAAGCC |
| | | CTTCGAGAGGGACATCAGCACCGAGATCTACCAGGCC |
| | | GGCAGCAAGCCCTGCAACGGCGTGGAGGGCTTCAACT |
| | | GCTACTTCCCCCTGCAGAGCTACGGCTTCCAGCCCAC |
| | | CAACGGCGTGGGCTACCAGCCCTACAGAGTGGTGGTG |
| | | CTGAGCTTCGAGCTGCTGCACGCCCCCGCCACCGTGT |
| | | GCGGCCCCAAGAAGAGCACGAACCTCGTGAAGAACAA |
| | | GTGCGTGAACTTCAACTTCAACGGCGGCAGCTCCGGC |
| | | GGCTACGAGCAGTACATCAAGTGGCCCTGGTACATCT |
| | | GGCTGGGCTTCATCGCCGGCCTGATCGCCATCGTGAT |
| | | GGTGACCATCATGCTGTGCTGCATGACCAGCTGCTGC |
| | | AGCTGCCTGAAGGGCTGCTGCAGCTGCGGCAGCTGCT |
| | | GCAAGTTCGACGAGGACGACAGCGAGCCCGTGCTGAA |
| | | GGGCGTGAAGCTGCACTACACCTAATGA |
| RBD-TM ‘Delta- | 21 | MFVFLVLLPLVSSQCVNLTTGIYQTSNFRVQPTESIV |
| plus’ variant | | RFPNITNLCPFGEVFNATRFASVYAWNRKRISNCVAD |
| Amino acid | | YSVLYNSASFSTFKCYGVSPTKLNDLCFTNVYADSFV |
| sequence | | IRGDEVRQIAPGQTGNIADYNYKLPDDFTGCVIAWNS |
| Signal peptide | | NNLDSKVGGNYNYRYRLFRKSNLKPFERDISTEIYQA |
| Mutations K417N, | | GSKPCNGVEGFNCYFPLQSYGFQPTNGVGYQPYRVVV |
| L452R & T478K | | LSFELLHAPATVCGPKKSTNLVKNKCVNFNFNGGSSG |
| Linker | | GYEQYIKWPWYIWLGFIAGLIAIVMVTIMLCCMTSCC |
| C-terminal domain | | SCLKGCCSCGSCCKFDEDDSEPVLKGVKLHYT |
| comprising | | |
| transmembrane | | |
| and cytoplasmic | | |
| region | | |
| Stop codons | | |
| RBD-TM ‘Delta- | 22 | AUGUUCGUGUUCCUGGUGCUGCUGCCCCUGGUGAGCA |
| plus’ variant | | GCCAGUGCGUGAACCUGACCACCGGCAUCUACCAGAC |
| mRNA sequence | | CAGCAACUUCAGGGUGCAGCCCACCGAGAGCAUCGUG |
| Signal peptide | | AGGUUCCCCAACAUCACCAACCUGUGCCCCUUCGGCG |
| Mutations K417N, | | AGGUGUUCAACGCCACCAGGUUCGCGAGCGUGUACGC |
| L452R & T478K | | CUGGAACAGGAAGAGGAUCAGCAACUGCGUGGCCGAC |
| Linker | | UACAGCGUGCUGUACAACAGCGCCAGCUUCAGCACCU |
| C-terminal domain | | UCAAGUGCUACGGCGUGAGCCCCACCAAGUUAAACGA |
| comprising | | CCUGUGCUUCACCAACGUGUACGCCGACAGCUUCGUG |
| transmembrane | | AUCAGGGGCGACGAGGUGAGGCAGAUCGCCCCCGGCC |
| and cytoplasmic | | AGACCGGCAACAUCGCCGACUACAACUACAAGCUGCC |
| region | | CGACGACUUCACCGGCUGCGUGAUCGCCUGGAACAGC |
| Stop codons | | AACAACCUGGACAGCAAGGUGGGCGGCAACUACAACU |
| | | ACAGGUACAGGCUGUUCAGGAAGAGCAACCUGAAGCC |
| | | CUUCGAGAGGGACAUCAGCACCGAGAUCUACCAGGCC |
| | | GGCAGCAAGCCCUGCAACGGCGUGGAGGGCUUCAACU |
| | | GCUACUUCCCCCUGCAGAGCUACGGCUUCCAGCCCAC |
| | | CAACGGCGUGGGCUACCAGCCCUACAGAGUGGUGGUG |
| | | CUGAGCUUCGAGCUGCUGCACGCCCCCGCCACCGUGU |
| | | GCGGCCCCAAGAAGAGCACGAACCUCGUGAAGAACAA |
| | | GUGCGUGAACUUCAACUUCAACGGCGGCAGCUCCGGC |
| | | GGCUACGAGCAGUACAUCAAGUGGCCCUGGUACAUCU |
| | | GGCUGGGCUUCAUCGCCGGCCUGAUCGCCAUCGUGAU |
| | | GGUGACCAUCAUGCUGUGCUGCAUGACCAGCUGCUGC |
| | | AGCUGCCUGAAGGGCUGCUGCAGCUGCGGCAGCUGCU |
| | | GCAAGUUCGACGAGGACGACAGCGAGCCCGUGCUGAA |
| | | GGGCGUGAAGCUGCACUACACCUAAUGA |
| RBD-TM ‘Delta- | 23 | ATGTTCGTGTTCCTGGTGCTGCTGCCCCTGGTGAGCA |
| plus’ variant | | GCCAGTGCGTGAACCTGACCACCGGCATCTACCAGAC |
| DNA sequence | | CAGCAACTTCAGGGTGCAGCCCACCGAGAGCATCGTG |
| Signal peptide | | AGGTTCCCCAACATCACCAACCTGTGCCCCTTCGGCG |
| Mutations K417N, | | AGGTGTTCAACGCCACCAGGTTCGCGAGCGTGTACGC |
| L452R & T478K | | CTGGAACAGGAAGAGGATCAGCAACTGCGTGGCCGAC |
| Linker | | TACAGCGTGCTGTACAACAGCGCCAGCTTCAGCACCT |
| C-terminal domain | | TCAAGTGCTACGGCGTGAGCCCCACCAAGTTAAACGA |
| comprising | | CCTGTGCTTCACCAACGTGTACGCCGACAGCTTCGTG |
| transmembrane | | ATCAGGGGCGACGAGGTGAGGCAGATCGCCCCCGGCC |
| and cytoplasmic | | AGACCGGCAACATCGCCGACTACAACTACAAGCTGCC |
| region | | CGACGACTTCACCGGCTGCGTGATCGCCTGGAACAGC |
| Stop codons | | AACAACCTGGACAGCAAGGTGGGCGGCAACTACAACT |
| | | ACAGGTACAGGCTGTTCAGGAAGAGCAACCTGAAGCC |
| | | CTTCGAGAGGGACATCAGCACCGAGATCTACCAGGCC |
| | | GGCAGCAAGCCCTGCAACGGCGTGGAGGGCTTCAACT |
| | | GCTACTTCCCCCTGCAGAGCTACGGCTTCCAGCCCAC |
| | | CAACGGCGTGGGCTACCAGCCCTACAGAGTGGTGGTG |
| | | CTGAGCTTCGAGCTGCTGCACGCCCCCGCCACCGTGT |
| | | GCGGCCCCAAGAAGAGCACGAACCTCGTGAAGAACAA |
| | | GTGCGTGAACTTCAACTTCAACGGCGGCAGCTCCGGC |
| | | GGCTACGAGCAGTACATCAAGTGGCCCTGGTACATCT |
| | | GGCTGGGCTTCATCGCCGGCCTGATCGCCATCGTGAT |
| | | GGTGACCATCATGCTGTGCTGCATGACCAGCTGCTGC |
| | | AGCTGCCTGAAGGGCTGCTGCAGCTGCGGCAGCTGCT |
| | | GCAAGTTCGACGAGGACGACAGCGAGCCCGTGCTGAA |
| | | GGGCGTGAAGCTGCACTACACCTAATGA |
| RBD-TM Omicron | 24 | MFVFLVLLPLVSSQCVNLTTGIYQTSNFRVQPTESIV |
| BA.1 variant | | RFPNITNLCPFDEVFNATRFASVYAWNRKRISNCVAD |
| Amino acid | | YSVLYNLAPFFTFKCYGVSPTKLNDLCFTNVYADSFV |
| sequence | | IRGDEVRQIAPGQTGNIADYNYKLPDDFTGCVIAWNS |
| Signal peptide | | NKLDSKVSGNYNYLYRLFRKSNLKPFERDISTEIYQA |
| Mutations G339D, | | GNKPCNGVAGFNCYFPLRSYSFRPTYGVGHQPYRVVV |
| S371L, S373P, | | LSFELLHAPATVCGPKKSTNLVKNKCVNFNFNGGSSG |
| S375E, K417N, | | GYEQYIKWPWYIWLGFIAGLIAIVMVTIMLCCMTSCC |
| N440K, G446S, | | SCLKGCCSCGSCCKFDEDDSEPVLKGVKLHYT |
| S477N, T478K, | | |
| E484A, Q493R, | | |
| G496S, Q498R, | | |
| N501Y & Y505H | | |
| Linker | | |
| C-terminal domain | | |
| comprising | | |
| transmembrane | | |
| and cytoplasmic | | |
| region | | |
| Stop codons | | |
| RBD-TM Omicron | 25 | AUGUUCGUGUUCCUCGUCCUCCUGCCCCUGGUGUCGU |
| BA.1 variant | | CGCAGUGCGUGAACCUCACCACCGGCAUCUACCAGAC |
| mRNA sequence | | CAGCAACUUCCGGGUGCAGCCCACGGAGAGCAUCGUC |
| Signal peptide | | CGCUUCCCGAACAUCACCAACCUCUGCCCCUUCGACG |
| Mutations G339D, | | AGGUGUUCAACGCGACGCGCUUCGCCAGCGUCUACGC |
| S371L, S373P, | | CUGGAACCGGAAGCGGAUCUCCAACUGCGUCGCGGAC |
| S375F, K417N, | | UACUCCGUCCUGUACAACCUCGCCCCAUUCUUCACGU |
| N440K, G446S, | | UCAAGUGCUACGGGGUGUCGCCGACGAAGCUCAACGA |
| S477N, T478K, | | CCUGUGCUUCACGAACGUCUACGCAGACUCCUUCGUC |
| E484A, Q493R, | | AUCCGGGGCGACGAGGUGCGCCAGAUCGCCCCAGGAC |
| G496S, Q498R, | | AGACCGGGAACAUCGCGGACUACAACUACAAGCUCCC |
| N501Y & Y505H | | CGACGACUUCACCGGGUGCGUCAUCGCCUGGAACAGC |
| Linker | | AACAAGCUCGACUCGAAGGUCUCGGGGAACUACAACU |
| C-terminal domain | | ACCUGUACAGGCUCUUCCGGAAGUCGAACCUGAAGCC |
| comprising | | AUUCGAGCGCGACAUCUCGACCGAGAUCUACCAGGCG |
| transmembrane | | GGCAACAAGCCAUGCAACGGGGUGGCCGGCUUCAACU |
| and cytoplasmic | | GCUACUUCCCGCUCCGCAGCUACUCGUUCCGCCCAAC |
| region | | CUACGGCGUGGGGCACCAGCCGUACAGGGUGGUCGUG |
| Stop codons | | CUGUCCUUCGAGCUGCUGCACGCGCCGGCCACCGUCU |
| | | GCGGCCCAAAGAAGUCGACGAACCUGGUCAAGAACAA |
| | | GUGCGUCAACUUCAACUUCAACGGGGGCAGCUCGGGG |
| | | GGGUACGAGCAGUACAUCAAGUGGCCGUGGUACAUCU |
| | | GGCUGGGGUUCAUCGCCGGGCUCAUCGCGAUCGUGAU |
| | | GGUGACGAUCAUGCUGUGCUGCAUGACCAGCUGCUGC |
| | | UCGUGCCUCAAGGGGUGCUGCUCGUGCGGAUCGUGCU |
| | | GCAAGUUCGACGAGGACGACUCGGAGCCCGUCCUCAA |
| | | GGGGGUCAAGCUCCACUACACCUGAUAA |
| RBD-TM Omicron | 26 | ATGTTCGTGTTCCTCGTCCTCCTGCCCCTGGTGTCGT |
| BA.1 variant | | CGCAGTGCGTGAACCTCACCACCGGCATCTACCAGAC |
| DNA sequence | | CAGCAACTTCCGGGTGCAGCCCACGGAGAGCATCGTC |
| Signal peptide | | CGCTTCCCGAACATCACCAACCTCTGCCCCTTCGACG |
| Mutations G339D, | | AGGTGTTCAACGCGACGCGCTTCGCCAGCGTCTACGC |
| S371L, S373?, | | CTGGAACCGGAAGCGGATCTCCAACTGCGTCGCGGAC |
| S375E, K417N, | | TACTCCGTCCTGTACAACCTCGCCCCATTCTTCACGT |
| N440K, G446S, | | TCAAGTGCTACGGGGTGTCGCCGACGAAGCTCAACGA |
| S477N, T478K, | | CCTGTGCTTCACGAACGTCTACGCAGACTCCTTCGTC |
| E484A, Q493R, | | ATCCGGGGCGACGAGGTGCGCCAGATCGCCCCAGGAC |
| G496S, Q498R, | | AGACCGGGAACATCGCGGACTACAACTACAAGCTCCC |
| N501Y & Y505H | | CGACGACTTCACCGGGTGCGTCATCGCCTGGAACAGC |
| Linker | | AACAAGCTCGACTCGAAGGTCTCGGGGAACTACAACT |
| C-terminal domain | | ACCTGTACAGGCTCTTCCGGAAGTCGAACCTGAAGCC |
| comprising | | ATTCGAGCGCGACATCTCGACCGAGATCTACCAGGCG |
| transmembrane | | GGCAACAAGCCATGCAACGGGGTGGCCGGCTTCAACT |
| and cytoplasmic | | GCTACTTCCCGCTCCGCAGCTACTCGTTCCGCCCAAC |
| region | | CTACGGCGTGGGGCACCAGCCGTACAGGGTGGTCGTG |
| Stop codons | | CTGTCCTTCGAGCTGCTGCACGCGCCGGCCACCGTCT |
| | | GCGGCCCAAAGAAGTCGACGAACCTGGTCAAGAACAA |
| | | GTGCGTCAACTTCAACTTCAACGGGGGCAGCTCGGGG |
| | | GGGTACGAGCAGTACATCAAGTGGCCGTGGTACATCT |
| | | GGCTGGGGTTCATCGCCGGGCTCATCGCGATCGTGAT |
| | | GGTGACGATCATGCTGTGCTGCATGACCAGCTGCTGC |
| | | TCGTGCCTCAAGGGGTGCTGCTCGTGCGGATCGTGCT |
| | | GCAAGTTCGACGAGGACGACTCGGAGCCCGTCCTCAA |
| | | GGGGGTCAAGCTCCACTACACCTGATAA |
| RBD-TM Omicron | 27 | MFVFLVLLPLVSSQCVNLTTGIYQTSNFRVQPTESIV |
| BA.2 variant | | RFPNITNLCPFDEVFNATRFASVYAWNRKRISNCVAD |
| Amino acid | | YSVLYNFAPFFAFKCYGVSPTKLNDLCFTNVYADSFV |
| sequence | | IRGNEVSQIAPGQTGNIADYNYKLPDDFTGCVIAWNS |
| Signal peptide | | NKLDSKVGGNYNYLYRLFRKSNLKPFERDISTEIYQA |
| Mutations G339D, | | GNKPCNGVAGFNCYFPLRSYGFRPTYGVGHQPYRVVV |
| S371E, S373P, | | LSFELLHAPATVCGPKKSTNLVKNKCVNFNFNGGSSG |
| S375F, T376A, | | GYEQYIKWPWYIWLGFIAGLIAIVMVTIMLCCMTSCC |
| D405N, R408, | | SCLKGCCSCGSCCKFDEDDSEPVLKGVKLHYT |
| K417N, N440K, | | |
| S477N, T478K, | | |
| E484A, Q493R, | | |
| Q498R, N501Y & | | |
| Y505H | | |
| Linker | | |
| C-terminal domain | | |
| comprising | | |
| transmembrane | | |
| and cytoplasmic | | |
| region | | |
| Stop codons | | |
| RBD-TM Omicron | 28 | AUGUUCGUGUUCCUCGUCCUCCUGCCCCUGGUGUCGU |
| BA.2 variant | | CGCAGUGCGUGAACCUCACCACCGGCAUCUACCAGAC |
| mRNA sequence | | CAGCAACUUCCGGGUGCAGCCCACGGAGAGCAUCGUC |
| Signal peptide | | CGCUUCCCGAACAUCACCAACCUCUGCCCCUUCGACG |
| Mutations G339D, | | AGGUGUUCAACGCGACGCGCUUCGCCAGCGUCUACGC |
| S371E, S373P, | | CUGGAACCGGAAGCGGAUCUCCAACUGCGUCGCGGAC |
| S375F, T376A, | | UACUCCGUCCUGUACAACUUCGCCCCAUUCUUCGCGU |
| D405N, R408S, | | UCAAGUGCUACGGGGUGUCGCCGACGAAGCUCAACGA |
| K417N, N440K, | | CCUGUGCUUCACGAACGUCUACGCAGACUCCUUCGUC |
| S477N, T478K, | | AUCCGGGGCAACGAGGUGAGCCAGAUCGCCCCAGGAC |
| E484A, Q493R, | | AGACCGGGAACAUCGCGGACUACAACUACAAGCUCCC |
| Q498R, N501Y & | | CGACGACUUCACCGGGUGCGUCAUCGCCUGGAACAGC |
| Y505H | | AACAAGCUCGACUCGAAGGUCGGGGGGAACUACAACU |
| Linker | | ACCUGUACAGGCUCUUCCGGAAGUCGAACCUGAAGCC |
| C-terminal domain | | AUUCGAGCGCGACAUCUCGACCGAGAUCUACCAGGCG |
| comprising | | GGCAACAAGCCAUGCAACGGGGUGGCCGGCUUCAACU |
| transmembrane | | GCUACUUCCCGCUCCGCAGCUACGGGUUCCGCCCAAC |
| and cytoplasmic | | CUACGGCGUGGGGCACCAGCCGUACAGGGUGGUCGUG |
| region | | CUGUCCUUCGAGCUGCUGCACGCGCCGGCCACCGUCU |
| Stop codons | | GCGGCCCAAAGAAGUCGACGAACCUGGUCAAGAACAA |
| | | GUGCGUCAACUUCAACUUCAACGGGGGCAGCUCGGGG |
| | | GGGUACGAGCAGUACAUCAAGUGGCCGUGGUACAUCU |
| | | GGCUGGGGUUCAUCGCCGGGCUCAUCGCGAUCGUGAU |
| | | GGUGACGAUCAUGCUGUGCUGCAUGACCAGCUGCUGC |
| | | UCGUGCCUCAAGGGGUGCUGCUCGUGCGGAUCGUGCU |
| | | GCAAGUUCGACGAGGACGACUCGGAGCCCGUCCUCAA |
| | | GGGGGUCAAGCUCCACUACACCUGAUAA |
| RBD-TM Omicron | 29 | ATGTTCGTGTTCCTCGTCCTCCTGCCCCTGGTGTCGT |
| BA.2 variant | | CGCAGTGCGTGAACCTCACCACCGGCATCTACCAGAC |
| DNA sequence | | CAGCAACTTCCGGGTGCAGCCCACGGAGAGCATCGTC |
| Signal peptide | | CGCTTCCCGAACATCACCAACCTCTGCCCCTTCGACG |
| Mutations G339D, | | AGGTGTTCAACGCGACGCGCTTCGCCAGCGTCTACGC |
| S371E, S373P, | | CTGGAACCGGAAGCGGATCTCCAACTGCGTCGCGGAC |
| S375F, T376A, | | TACTCCGTCCTGTACAACTTCGCCCCATTCTTCGCGT |
| D405N, R408S, | | TCAAGTGCTACGGGGTGTCGCCGACGAAGCTCAACGA |
| K417N, N440K, | | CCTGTGCTTCACGAACGTCTACGCAGACTCCTTCGTC |
| S477N, T478K, | | ATCCGGGGCAACGAGGTGAGCCAGATCGCCCCAGGAC |
| E484A, Q493R, | | AGACCGGGAACATCGCGGACTACAACTACAAGCTCCC |
| Q498R, N501Y & | | CGACGACTTCACCGGGTGCGTCATCGCCTGGAACAGC |
| Y505H | | AACAAGCTCGACTCGAAGGTCGGGGGGAACTACAACT |
| Linker | | ACCTGTACAGGCTCTTCCGGAAGTCGAACCTGAAGCC |
| C-terminal domain | | ATTCGAGCGCGACATCTCGACCGAGATCTACCAGGCG |
| comprising | | GGCAACAAGCCATGCAACGGGGTGGCCGGCTTCAACT |
| transmembrane | | GCTACTTCCCGCTCCGCAGCTACGGGTTCCGCCCAAC |
| and cytoplasmic | | CTACGGCGTGGGGCACCAGCCGTACAGGGTGGTCGTG |
| region | | CTGTCCTTCGAGCTGCTGCACGCGCCGGCCACCGTCT |
| Stop codons | | GCGGCCCAAAGAAGTCGACGAACCTGGTCAAGAACAA |
| | | GTGCGTCAACTTCAACTTCAACGGGGGCAGCTCGGGG |
| | | GGGTACGAGCAGTACATCAAGTGGCCGTGGTACATCT |
| | | GGCTGGGGTTCATCGCCGGGCTCATCGCGATCGTGAT |
| | | GGTGACGATCATGCTGTGCTGCATGACCAGCTGCTGC |
| | | TCGTGCCTCAAGGGGTGCTGCTCGTGCGGATCGTGCT |
| | | GCAAGTTCGACGAGGACGACTCGGAGCCCGTCCTCAA |
| | | GGGGGTCAAGCTCCACTACACCTGATAA |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The general chemical terms used in the formulae herein have their usual meaning.

The present invention relates to nucleic acid molecules (polynucleotides), and compositions comprising the same, for use in eliciting an immune response to a coronavirus.

### RBD of Spike Protein

Current approaches for raising immune responses to coronaviruses are typically based on the administration of whole spike proteins from the coronavirus, or nucleic acids encoding the same. Administration of such proteins or nucleic acids therefore elicits an immune response to the whole spike protein of the virus. A limitation of this approach is that the immune response may result in the generation of a subset of antibodies which are non-neutralising. Although not wishing to be bound by theory, the inventors believe that such non-neutralising antibodies may lead to antibody-enhanced disease and in the elderly, may result in ineffective vaccination if the subjects are primed to induce antibodies against epitopes outside key regions of the spike protein.

The present invention is based on the design of alternative vaccine candidates, in which a polynucleotide is provided for expression of only a part of the coronavirus spike protein, being the receptor-binding domain (RBD). More specifically, the inventors propose a vaccine that comprises a polynucleotide encoding a membrane-anchored form of the RBD, (referred to as RBD-TM herein). Surprisingly, the inventors have demonstrated that vaccination with RBD-TM mRNA elicits a protective immune response to SARS-CoV-2 despite the fact that the RBD may not be presented in a similar conformation to the naturally occuring trimeric whole spike structure.

It is believed that the approach of the present invention enables the delivery of both lower doses of mRNA and of the delivery system (eg lipid nanoparticles). Reducing the amount of both mRNA and lipid nanoparticle in vaccine formulations is believed to result in lower reactogenicity, providing for vaccines which are well tolerated while also providing sufficient mRNA at a lower dose. A further advantage is that the vaccines of the invention are adaptable to facilitate delivery of mRNAs encoding multiple RBD-TM sequences (ie a multivalent vaccine) while keeping the total dose of mRNA within acceptable limits.

Spike (S) is a structural glycoprotein expressed at the SARS-CoV-2 (2019-nCoV) surface and is a critical determinant of the viral host and tissue tropism. SARS-CoV-2 S mediates the virus entry into the target cells upon ACE2 receptor binding and has therefore been the target of the development of therapeutic drug design. Elevated Anti-SARS-CoV-2 S antibody titers are detected in COVID-19 patients' sera, rising approximately ten days after symptoms onset. These observations also make SARS-CoV-2 S protein an attractive tool for early diagnosis.

Spike antigenicity is also highlighted by the observation of Spike-specific CD4+ and CD8+ T cells in blood samples from recovered COVID-19 patients. As a consequence, SARS-CoV-2 S has been the main target for prophylactic vaccination strategies.

Most of the current knowledge about SARS-CoV-2 S protein is based on analogies with findings on the previously identified SARS-CoV S protein. Clove-shaped trimers of Spike proteins form large surface protrusions that give the coronaviruses the appearance of having a crown. Each Spike protomer contains three segments: a large ectodomain, a transmembrane anchor (TM), and a short intracellular tail (IC). The Spike ectodomain contains three critical elements:
the S1 subunit contains an N-terminal (S1-NTD) and a C-terminal (S1-CTD) subdomains. The S1 “closed” conformation exerts a physical constraint on the S2 subunit until specific proteases cleave the S1/S2 and S2′ sites;
the RBD (receptor binding domain) is located in the S1-CTD region and is buried in the inner S1 head-trimer. The S1 “open” conformation is expected to be necessary for binding to the ACE2 receptor at the surface of host target cells;
the S2 subunit forms a trimeric stalk. It contains a fusion peptide (FP) and two heptad repeats (HR1 and HR2), which operate the fusion of viral and host membranes.

Two cleavage sites at the S1 and S2 boundary (S1/S2) and in the S2 domain (S2′) play an essential role in the viral entry into host target cells.

The Spike protein exists in two structurally distinct conformations: pre-fusion and post-fusion. In its pre-fusion state, Spike is a “closed” trimer and RBDs are buried in the inner S1 head-trimer, at the interface between each protomer. This “closed” conformation exerts a physical constraint on the S2 subunit until specific proteases cleave the S1/S2 and S2′ sites. The exact mechanisms driving the opening of an S1-CTD domain and the subsequent exposition of RBD so that it can bind the ACE2 receptor are not elucidated yet. It has been proposed that the S protein is cleaved into S1 and S2 subunits by proteases, including furin, the host surface-associated transmembrane protease serine 2 (TMPRSS2), and the endocytic cathepsin L. S1 binds to ACE2 through its RBD, and S2 is further cleaved and activated by TMPRSS2 and/or cathepsin L. Together these actions result in host-viral membrane fusion and release of the viral RNA genome into the host cell cytoplasm.

### Coronavirus

“Coronavirus” as used herein refers to members of the subfamily Coronavirinae in the family Coronaviridae and the order Nidovirales (International Committee on Taxonomy of Viruses). This subfamily consists of four genera, Alphacoronavirus, Betacoronavirus, Gammacoronavirus and Deltacoronavirus, on the basis of their phylogenetic relationships and genomic structures. Subgroup clusters are labeled as 1a and 1b for the Alphacoronavirus and 2a, 2b, 2c, and 2d for the Betacoronavirus. The alphacoronaviruses and betacoronaviruses infect only mammals. The gammacoronaviruses and deltacoronaviruses infect birds, but some of them can also infect mammals. Alphacoronaviruses and betacoronaviruses usually cause respiratory illness in humans and gastroenteritis in animals. The three highly pathogenic viruses, SARS-CoV, MERS-CoV and SARS-CoV-2, cause severe respiratory syndrome in humans, and the other four human coronaviruses (HCoV-NL63, HCoV-229E, HCoV-OC43 and HKU1) induce only mild upper respiratory diseases in immunocompetent hosts, although some of them can cause severe infections in infants, young children and elderly individuals. Alphacoronaviruses and betacoronaviruses can pose a heavy disease burden on livestock; these viruses include porcine transmissible gastroenteritis virus, porcine enteric diarrhoea virus (PEDV) and the recently emerged swine acute diarrhoea syndrome coronavirus (SADS-CoV). On the basis of current sequence databases, all human coronaviruses have animal origins: SARS-CoV, MERS-CoV, SARS-CoV-2, HCoV-NL63 and HCoV-229E are considered to have originated in bats; HCoV-OC43 and HKU1 likely originated from rodents.

The coronaviruses include antigenic groups I, II, and III. Nonlimiting examples of coronaviruses include SARS coronavirus, MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as any others described herein, and including those referred to in Cui, et al. Nature Reviews Microbiology volume 17, pages 181-192 (2019), and Shereen et al. Journal of Advanced Research, Volume 24, July 2020 (published online 16 Mar. 2020), Pages 91-98.

Non-limiting examples of a subgroup 1a coronavirus include FCov.FIPV.79.1146.VR.2202 (GenBank Accession No. NV_007025), transmissible gastroenteritis virus (TGEV) (GenBank Accession No. NC_002306; GenBank Accession No. Q811789.2; GenBank Accession No. DQ811786.2; GenBank Accession No. DQ811788.1; GenBank Accession No. DQ811785.1; GenBank Accession No. X52157.1; GenBank Accession No. AJ011482.1; GenBank Accession No. KC962433.1; GenBank Accession No. AJ271965.2; GenBank Accession No. JQ693060.1; GenBank Accession No. KC609371.1; GenBank Accession No. JQ693060.1; GenBank Accession No. JQ693059.1; GenBank Accession No. JQ693058.1; GenBank Accession No. JQ693057.1; GenBank Accession No. JQ693052.1; GenBank Accession No. JQ693051.1; GenBank Accession No. JQ693050.1), porcine reproductive and respiratory syndrome virus (PRRSV) (GenBank Accession No. NC_001961.1; GenBank Accession No. DQ811787), as well as any other subgroup 1a coronavirus now known (e.g., as can be found in the GenBank® Database) or later identified, and any combination thereof.

Non-limiting examples of a subgroup 1b coronavirus include BtCoV.1A.AFCD62 (GenBank Accession No. NC_010437), BtCoV.1B.AFCD307 (GenBank Accession No. NC_010436), BtCov.HKU8.AFCD77 (GenBank Accession No. NC_010438), BtCoV.512.2005 (GenBank Accession No. DQ648858), porcine epidemic diarrhea virus PEDV.CV777 (GenBank Accession No. NC_003436, GenBank Accession No. DQ355224.1, GenBank Accession No. DQ355223.1, GenBank Accession No. DQ355221.1, GenBank Accession No. JN601062.1, GenBank Accession No. N601061.1, GenBank Accession No. JN601060.1, GenBank Accession No. JN601059.1, GenBank Accession No. JN601058.1, GenBank Accession No. JN601057.1, GenBank Accession No. JN601056.1, GenBank Accession No. JN601055.1, GenBank Accession No. JN601054.1, GenBank Accession No. JN601053.1, GenBank Accession No. JN601052.1, GenBank Accession No. JN400902.1, GenBank Accession No. JN547395.1, GenBank Accession No. FJ687473.1, GenBank Accession No. FJ687472.1, GenBank Accession No. FJ687471.1, GenBank Accession No. FJ687470.1, GenBank Accession No. FJ687469.1, GenBank Accession No. FJ687468.1, GenBank Accession No. FJ687467.1, GenBank Accession No. FJ687466.1, GenBank Accession No. FJ687465.1, GenBank Accession No. FJ687464.1, GenBank Accession No. FJ687463.1, GenBank Accession No. FJ687462.1, GenBank Accession No. FJ687461.1, GenBank Accession No. FJ687460.1, GenBank Accession No. FJ687459.1, GenBank Accession No. FJ687458.1, GenBank Accession No. FJ687457.1, GenBank Accession No. FJ687456.1, GenBank Accession No. FJ687455.1, GenBank Accession No. FJ687454.1, GenBank Accession No. FJ687453 GenBank Accession No. FJ687452.1, GenBank Accession No. FJ687451.1, GenBank Accession No. FJ687450.1, GenBank Accession No. FJ687449.1, GenBank Accession No. AF500215.1, GenBank Accession No. KF476061.1, GenBank Accession No. KF476060.1, GenBank Accession No. KF476059.1, GenBank Accession No. KF476058.1, GenBank Accession No. KF476057.1, GenBank Accession No. KF476056.1, GenBank Accession No. KF476055.1, GenBank Accession No. KF476054.1, GenBank Accession No. KF476053.1, GenBank Accession No. KF476052.1, GenBank Accession No. KF476051.1, GenBank Accession No. KF476050.1, GenBank Accession No. KF476049.1, GenBank Accession No. KF476048.1, GenBank Accession No. KF177258.1, GenBank Accession No. KF177257.1, GenBank Accession No. KF177256.1, GenBank Accession No. KF177255.1), HCoV.229E (GenBank Accession No. NC_002645), HCoV.NL63.Amsterdam.I (GenBank Accession No. NC_005831), BtCoV.HKU2.HK.298.2006 (GenBank Accession No. EF203066), BtCoV.HKU2.HK.33.2006 (GenBank Accession No. EF203067), BtCoV.HKU2.HK.46.2006 (GenBank Accession No. EF203065), BtCoV.HKU2.GD.430.2006 (GenBank Accession No. EF203064), as well as any other subgroup 1b coronavirus now known (e.g., as can be found in the GenBank® Database) or later identified, and any combination thereof.

Non-limiting examples of a subgroup 2a coronavirus include HCoV.HKU1.C.N5 (GenBank Accession No. DQ339101), MHV.A59 (GenBank Accession No. NC 001846), PHEV.VW572 (GenBank Accession No. NC 007732), HCoV.OC43.ATCC.VR.759 (GenBank Accession No. NC_005147), bovine enteric coronavirus (BCoV.ENT) (GenBank Accession No. NC_003045), as well as any other subgroup 2a coronavirus now known (e.g., as can be found in the GenBank® Database) or later identified, and any combination thereof.

Non-limiting examples of subgroup 2b coronaviruses include Bat SARS CoV (GenBank Accession No. FJ211859), SARS CoV (GenBank Accession No. FJ211860), SARS-CoV-2 (GenBank Accession No. NC_045512.2), BtSARS.HKU3.1 (GenBank Accession No. DQ022305), BtSARS.HKU3.2 (GenBank Accession No. DQ084199), BtSARS.HKU3.3 (GenBank Accession No. DQ084200), BtSARS.Rm1 (GenBank Accession No. DQ412043), BtCoV.279.2005 (GenBank Accession No. DQ648857), BtSARS.Rf1 (GenBank Accession No. DQ412042), BtCoV.273.2005 (GenBank Accession No. DQ648856), BtSARS.Rp3 (GenBank Accession No. DQ071615), SARS CoV.A022 (GenBank Accession No. AY686863), SARSCoV.CUHK-W1 (GenBank Accession No. AY278554), SARSCoV.GDO1 (GenBank Accession No. AY278489), SARSCoV.HC.SZ.61.03 (GenBank Accession No. AY515512), SARSCoV.SZ16 (GenBank Accession No. AY304488), SARSCoV.Urbani (GenBank Accession No. AY278741), SARSCoV.civet010 (GenBank Accession No. AY572035), and SARSCoV.MA.15 (GenBank Accession No. DQ497008), Rs SHC014 (GenBank® Accession No. KC881005), Rs3367 (GenBank® Accession No. KC881006), WiV1 S (GenBank® Accession No. KC881007) as well as any other subgroup 2b coronavirus now known (e.g., as can be found in the GenBank® Database) or later identified, and any combination thereof.

Non-limiting examples of subgroup 2c coronaviruses include: Middle East respiratory syndrome coronavirus isolate Riyadh_2_2012 (GenBank Accession No. KF600652.1), Middle East respiratory syndrome coronavirus isolate Al-Hasa_18_2013 (GenBank Accession No. KF600651.1), Middle East respiratory syndrome coronavirus isolate AI-Hasa_17_2013 (GenBank Accession No. KF600647.1), Middle East respiratory syndrome coronavirus isolate Al-Hasa_15_2013 (GenBank Accession No. KF600645.1), Middle East respiratory syndrome coronavirus isolate Al-Hasa_16_2013 (GenBank Accession No. KF600644.1), Middle East respiratory syndrome coronavirus isolate Al-Hasa_21_2013 (GenBank Accession No. KF600634), Middle East respiratory syndrome coronavirus isolate Al-Hasa_19_2013 (GenBank Accession No. KF600632), Middle East respiratory syndrome coronavirus isolate Buraidah_1_2013 (GenBank Accession No. KF600630.1), Middle East respiratory syndrome coronavirus isolate Hafr-Al-Batin_1_2013 (GenBank Accession No. KF600628.1), Middle East respiratory syndrome coronavirus isolate Al-Hasa_12_2013 (GenBank Accession No. KF600627.1), Middle East respiratory syndrome coronavirus isolate Bisha_1_2012 (GenBank Accession No. KF600620.1), Middle East respiratory syndrome coronavirus isolate Riyadh_3_2013 (GenBank Accession No. KF600613.1), Middle East respiratory syndrome coronavirus isolate Riyadh_1_2012 (GenBank Accession No. KF600612.1), Middle East respiratory syndrome coronavirus isolate Al-Hasa_3_2013 (GenBank Accession No. KF186565.1), Middle East respiratory syndrome coronavirus isolate Al-Hasa_1_2013 (GenBank Accession No. KF186567.1), Middle East respiratory syndrome coronavirus isolate Al-Hasa_2_2013 (GenBank Accession No. KF186566.1), Middle East respiratory syndrome coronavirus isolate Al-Hasa_4_2013 (GenBank Accession No. KF186564.1), Middle East respiratory syndrome coronavirus (GenBank Accession No. KF192507.1), Betacoronavirus England 1-N1 (GenBank Accession No. NC_019843), MERS-CoV_SA-N1 (GenBank Accession No. KC667074), following isolates of Middle East Respiratory Syndrome Coronavirus (GenBank Accession No: KF600656.1, GenBank Accession No: KF600655.1, GenBank Accession No: KF600654.1, GenBank Accession No: KF600649.1, GenBank Accession No: KF600648.1, GenBank Accession No: KF600646.1, GenBank Accession No: KF600643.1, GenBank Accession No: KF600642.1, GenBank Accession No: KF600640.1, GenBank Accession No: KF600639.1, GenBank Accession No: KF600638.1, GenBank Accession No: KF600637.1, GenBank Accession No: KF600636.1, GenBank Accession No: KF600635.1, GenBank Accession No: KF600631.1, GenBank Accession No: KF600626.1, GenBank Accession No: KF600625.1, GenBank Accession No: KF600624.1, GenBank Accession No: KF600623.1, GenBank Accession No: KF600622.1, GenBank Accession No: KF600621.1, GenBank Accession No: KF600619.1, GenBank Accession No: KF600618.1, GenBank Accession No: KF600616.1, GenBank Accession No: KF600615.1, GenBank Accession No: KF600614.1, GenBank Accession No: KF600641.1, GenBank Accession No: KF600633.1, GenBank Accession No: KF600629.1, GenBank Accession No: KF600617.1), Coronavirus Neoromicia/PML-PHE1/RSA/2011 GenBank Accession: KC869678.2, Bat Coronavirus Taper/CII_KSA_287/Bisha/Saudi Arabia/GenBank Accession No: KF493885.1, Bat coronavirus Rhhar/CII_KSA_003/Bisha/Saudi Arabia/2013 GenBank Accession No: KF493888.1, Bat coronavirus Pikuh/CII_KSA_001/Riyadh/Saudi Arabia/2013 GenBank Accession No: KF493887.1, Bat coronavirus Rhhar/CII_KSA_002/Bisha/Saudi Arabia/2013 GenBank Accession No: KF493886.1, Bat Coronavirus Rhhar/CII_KSA_004/Bisha/Saudi Arabia/2013 GenBank Accession No: KF493884.1, BtCoV.HKU4.2 (GenBank Accession No. EF065506), BtCoV.HKU4.1 (GenBank Accession No. NC_009019), BtCoV.HKU4.3 (GenBank Accession No. EF065507), BtCoV.HKU4.4 (GenBank Accession No. EF065508), BtCoV 133.2005 (GenBank Accession No. NC 008315), BtCoV.HKU5.5 (GenBank Accession No. EF065512); BtCoV.HKU5.1 (GenBank Accession No. NC_009020), BtCoV.HKU5.2 (GenBank Accession No. EF065510), BtCoV.HKU5.3 (GenBank Accession No. EF065511), human betacoronavirus 2c Jordan-N3/2012 (GenBank Accession No. KC776174.1; human betacoronavirus 2c EMC/2012 (GenBank Accession No. JX869059.2), Pipistrellus bat coronavirus HKU5 isolates (GenBank Accession No: KC522089.1, GenBank Accession No: KC522088.1, GenBank Accession No: KC522087.1, GenBank Accession No: KC522086.1, GenBank Accession No: KC522085.1, GenBank Accession No: KC522084.1, GenBank Accession No: KC522083.1, GenBank Accession No: KC522082.1, GenBank Accession No: KC522081.1, GenBank Accession No: KC522080.1, GenBank Accession No: KC522079.1, GenBank Accession No: KC522078.1, GenBank Accession No: KC522077.1, GenBank Accession No: KC522076.1, GenBank Accession No: KC522075.1, GenBank Accession No: KC522104.1, GenBank Accession No: KC522104.1, GenBank Accession No: KC522103.1, GenBank Accession No: KC522102.1, GenBank Accession No: KC522101.1, GenBank Accession No: KC522100.1, GenBank Accession No: KC522099.1, GenBank Accession No: KC522098.1, GenBank Accession No: KC522097.1, GenBank Accession No: KC522096.1, GenBank Accession No: KC522095.1, GenBank Accession No: KC522094.1, GenBank Accession No: KC522093.1, GenBank Accession No: KC522092.1, GenBank Accession No: KC522091.1, GenBank Accession No: KC522090.1, GenBank Accession No: KC522119.1 GenBank Accession No: KC522118.1 GenBank Accession No: KC522117.1 GenBank Accession No: KC522116.1 GenBank Accession No: KC522115.1 GenBank Accession No: KC522114.1 GenBank Accession No: KC522113.1 GenBank Accession No: KC522112.1 GenBank Accession No: KC522111.1 GenBank Accession No: KC522110.1 GenBank Accession No: KC522109.1 GenBank Accession No: KC522108.1, GenBank Accession No: KC522107.1, GenBank Accession No: KC522106.1, GenBank Accession No: KC522105.1) Pipistrellus bat coronavirus HKU4 isolates (GenBank Accession No: KC522048.1, GenBank Accession No: KC522047.1, GenBank Accession No: KC522046.1, GenBank Accession No: KC522045.1, GenBank Accession No: KC522044.1, GenBank Accession No: KC522043.1, GenBank Accession No: KC522042.1, GenBank Accession No: KC522041.1, GenBank Accession No: KC522040.1 GenBank Accession No: KC522039.1, GenBank Accession No: KC522038.1, GenBank Accession No: KC522037.1, GenBank Accession No: KC522036.1, GenBank Accession No: KC522048.1 GenBank Accession No: KC522047.1 GenBank Accession No: KC522046.1 GenBank Accession No: KC522045.1 GenBank Accession No: KC522044.1 GenBank Accession No: KC522043.1 GenBank Accession No: KC522042.1 GenBank Accession No: KC522041.1 GenBank Accession No: KC522040.1, GenBank Accession No: KC522039.1 GenBank Accession No: KC522038.1 GenBank Accession No: KC522037.1 GenBank Accession No: KC522036.1, GenBank Accession No: KC522061.1 GenBank Accession No: KC522060.1 GenBank Accession No: KC522059.1 GenBank Accession No: KC522058.1 GenBank Accession No: KC522057.1 GenBank Accession No: KC522056.1 GenBank Accession No: KC522055.1 GenBank Accession No: KC522054.1 GenBank Accession No: KC522053.1 GenBank Accession No: KC522052.1 GenBank Accession No: KC522051.1 GenBank Accession No: KC522050.1 GenBank Accession No: KC522049.1 GenBank Accession No: KC522074.1, GenBank Accession No: KC522073.1 GenBank Accession No: KC522072.1 GenBank Accession No: KC522071.1 GenBank Accession No: KC522070.1 GenBank Accession No: KC522069.1 GenBank Accession No: KC522068.1 GenBank Accession No: KC522067.1, GenBank Accession No: KC522066.1 GenBank Accession No: KC522065.1 GenBank Accession No: KC522064.1, GenBank Accession No: KC522063.1, or GenBank Accession No: KC522062.1, as well as any other subgroup 2b coronavirus now known (e.g., as can be found in the GenBank® Database) or later identified, and any combination thereof.

Non-limiting examples of a subgroup 2d coronavirus include BtCoV.HKU9.2 (GenBank Accession No. EF065514), BtCoV.HKU9.1 (GenBank Accession No. NC_009021), BtCoV.HkU9.3 (GenBank Accession No. EF065515), BtCoV.HKU9.4 (GenBank Accession No. EF065516), as well as any other subgroup 2d coronavirus now known (e.g., as can be found in the GenBank® Database) or later identified, and any combination thereof.

Non-limiting examples of a subgroup 3 coronavirus include IBV.Beaudette.IBV.p65 (GenBank Accession No. DQ001339), as well as any other subgroup 3 coronavirus now known (e.g., as can be found in the GenBank® Database) or later identified, and any combination thereof.

The present invention contemplates the use of a polynucleotide encoding an RBD from any SARS-CoV-2 variant. Moreover, it will be appreciated from the disclosure herein, that the RBD sequence from a given variant may be useful for eliciting a suitable immune response in a subject, to the same or to different variants of SARS-CoV-2. For example, and as is demonstrated herein, an RBD sequence from the ancestral “wild-type” SARS-CoV-2 virus can be used to generate an immune response to one or more of the variants that have emerged since the first appearance of the SATS-CoV-2 virus. In another example, the RBD sequence from the Beta variant can be used to elicit an immune response to the ancestral wild-type train, or to any or the Alpha, Delta, Lambda, Beta, Gamma, Delta plus, mu or Omicron variants of the virus, in addition to the Beta variant.

It will also be appreciated that as the virus continues to evolve and acquired more mutations, that RBD sequences derived from more recently emerged variants may more useful for eliciting an immune response to more recently evolved forms of the virus.

In further embodiments, it will be appreciated that the vaccine compositions of the invention may comprise RBD mRNA sequences from more than one SARS-CoV-2 variant, so as to maximise the immune response to spike protein and to SARS-CoV-2 variants. In certain embodiments, the vaccine compositions of the invention may comprise an RBD mRNA sequence (preferably an RBD-TM mRNA sequence) from at least two different SARS-CoV-2 variants, at least three SARS-CoV-2 variants, at least four SARS-CoV-2 variants or more.

To date, the following variants have been described:

TABLE 2
examples of SARS-CoV-2 variants

| WHO label (original | | |
|---|---|---|
| designation) | In WT SARS-COV2 Spike* | In RBD-TM (current invention) # |
| Alpha (UK) | N501Y | N191Y |
| Alpha (UK) | E484K/N501Y | E174K/N191Y |
| Beta (Sth African) | K417N/E484K/N501Y | K107N/E174K/N191Y |
| Epsilon (United | L452R | L142R |
| States) | | |
| Kappa (Indian) | L452R/E484Q | L142R/E174Q |
| Delta (Indian) | L452R/T478K | L142R/T168K |
| Delta Plus (Indian) | K417N/L452R/T478K | K107N/L142R/T168K |
| Gamma | K417T/E484K/N501Y | K107T/E174K/N191Y |
| Omicron BA.1 | G339D/S371L/S373P/S375F/K417N/ | G29D/S61L/S63P/S65F/K107N/N130K/ |
| | N440K/G446S/S477N/T478K/E484A/ | G136S/S167N/T168K/E174A/Q183R/ |
| | Q493R/G496S/Q498R/N501Y/Y505H | G186S/Q188R/N191Y/Y195H |
| Omicron BA. 2 | G339D/S371F/S373P/S375F/T376A/ | G29D/S61F/S63P/S65F/T66A/D95N/ |
| | D405N/K417N/N440K/G446S/S477N/ | K107N/N130K/G136S/S167N/T168K/ |
| | T478K/E484A/Q493R/Q498R/N501Y/ | E174A/Q183R/Q188R/N191Y/Y195H |
| | Y505H | |
| Mu | R346K/E484K/N501Y | R36K/E174K/N191Y |

*(Source: UniProtKB - P0DTC2 (SPIKE_SARS2)
# numbering according to SEQ ID NO: 9 herein

### Nucleic Acids

The term “nucleic acid,” in its broadest sense, includes any compound and/or substance that comprise a polymer of nucleotides. These polymers are often referred to as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Typically, the polynucleotides of the invention are in the form of an mRNA molecule. As used herein, the term “messenger RNA” (mRNA) refers to any polynucleotide which encodes a polypeptide of interest and which is capable of being translated to produce the encoded polypeptide of interest in vitro, in vivo, in situ or ex vivo. The skilled artisan will appreciate that, except where otherwise noted, polynucleotide sequences set forth in the instant application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.”

The basic components of an mRNA molecule include at least a coding region, a 5′UTR, a 3′UTR, a 5′ cap and a poly-A tail. Polynucleotides of the present disclosure may function as mRNA but can be distinguished from wild-type mRNA in their functional and/or structural design features, which serve to overcome existing problems of effective polypeptide expression using nucleic-acid based therapeutics.

A “5′ untranslated region” (5′UTR) refers to a region of an mRNA that is directly upstream (i.e., 5′) from the start codon (i.e., the first codon of an mRNA transcript translated by a ribosome) that does not encode a polypeptide.

A “3′ untranslated region” (3′UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides.

The present invention also contemplates the use of one or more structural and/or chemical modifications or alterations which impart useful properties to the polynucleotide including, in some embodiments, the lack of a substantial induction of the innate immune response of a cell into which the polynucleotide is introduced. As such, modified mRNA molecules of the present invention may also be termed “mmRNA.” As used herein, a “structural” feature or modification is one in which two or more linked nucleotides are inserted, deleted, duplicated, inverted or randomized in a polynucleotide, primary construct or mmRNA without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

The mRNA molecules of the invention may also comprise an 5′ terminal cap. In some embodiments, the 5′ terminal cap is 7mG(5′)ppp(5′)NlmpNp although it will be appreciated that any number of different 5′ terminal caps commonly used in the art may be employed.

In some embodiments, the mRNA molecule comprises at least one chemical modification. The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” of they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphdioester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

The at least one chemical modification may be selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine. In some embodiments, the chemical modification is a N1-ethylpseudouridine. In some embodiments, polynucleotides includes a combination of at least two (e.g., 2, 3, 4 or more) of the aforementioned modified nucleobases.

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and In some embodiments, a modified nucleobase is a modified cytosine. nucleosides having a modified uridine include 5-cyano uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (mIG), 8-oxo-guanosine, 7-methyl-8-oxo-guanosine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the disclosure, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C orA+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). Any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). n some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridineor 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine (i.e., having the nucleobase deoxythymine), 1-methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine, 1-methyl-3-(3-amino-3-carboxypropyl)pseudouridine, 5-(isopentenylaminomethyl)uridine, 5-(isopentenylaminomethyl)-2-thio-uridine, α-thio-uridine, 2′-O-methyl-uridine, 5,2′-O-dimethyl-uridine, 2′-O-methyl-pseudouridine (Wm), 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, and 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine.

Exemplary nucleobases and nucleosides having a modified cytosine include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine (e.g., 5-iodo-cytidine), 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, α-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl)adenosine, 2-methylthio-N6-(cis-hydroxyisopentenyl)adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6,N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, α-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine.

Exemplary nucleobases and nucleosides having a modified guanine include inosine, 1-methyl-inosine, wyosine, methylwyosine, 4-demethyl-wyosine, isowyosine (imG2), wybutosine, peroxywybutosine, hydroxywybutosine, undermodified hydroxywybutosine, 7-deaza-guanosine, queuosine, epoxyqueuosine, galactosyl-queuosine (galQ), mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2,N2-dimethyl-guanosine, N2,7-dimethyl-guanosine, N2, N2,7-dimethyl-guanosine, 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2,7-dimethyl-2′-O-methyl-guanosine, 2′-O-methyl-inosine, 1,2′-O-dimethyl-inosine, 2′-O-ribosylguanosine (phosphate) (Gr(p)), 1-thio-guanosine, 06-methyl-guanosine, 2′-F-ara-guanosine, and 2′-F-guanosine.

In some embodiments, the RNA (e.g., mRNA) vaccines comprise a 5′UTR element, an optionally codon optimized open reading frame, and a 3′UTR element, a poly(A) sequence and/or a polyadenylation signal wherein the RNA is not chemically modified.

Polynucleotides of the present disclosure, in some embodiments, are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art-non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity, less than 90% sequence identity, less than 85% sequence identity, less than 80% sequence identity, or less than 75% sequence identity to a naturally-occurring or wild-type sequence (e.g., a naturally-occurring or wild-type mRNA sequence encoding a polypeptide or protein of interest (e.g., an antigenic protein or antigenic polypeptide)).

In some embodiments, a codon-optimized sequence shares between 65% and 85% (e.g., between about 67% and about 85%, or between about 67% and about 80%) sequence identity to a naturally-occurring sequence or a wild-type sequence (e.g., a naturally-occurring or wild-type mRNA sequence encoding a polypeptide or protein of interest (e.g., an antigenic protein or polypeptide)). In some embodiments, a codon-optimized sequence shares between 65% and 75%, or about 80% sequence identity to a naturally-occurring sequence or wild-type sequence (e.g., a naturally-occurring or wild-type mRNA sequence encoding a polypeptide or protein of interest (e.g., an antigenic protein or polypeptide)).

In some embodiments a codon-optimized RNA (e.g., mRNA) may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

Naturally-occurring eukaryotic mRNA molecules have been found to contain stabilizing elements, including, but not limited to untranslated regions (UTR) at their 5′-end (5′UTR) and/or at their 3′-end (3′UTR), in addition to other structural features, such as a 5′-cap structure or a 3′-poly(A) tail. Both the 5′UTR and the 3′UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing. The 3′-poly(A) tail is typically a stretch of adenine nucleotides added to the 3′-end of the transcribed mRNA. It can comprise up to about 400 adenine nucleotides. In some embodiments the length of the 3′-poly(A) tail may be an essential element with respect to the stability of the individual mRNA.

In some embodiments the RNA (e.g., mRNA) vaccine may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the RNA (e.g., mRNA) vaccines include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. It has been found that the synergistic effect of the combination of poly(A) and at least one histone stem-loop does not depend on the order of the elements or the length of the poly(A) sequence.

In some embodiments, the RNA (e.g., mRNA) vaccine does not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. Ideally, the inventive nucleic acid does not include an intron.

In some embodiments, the RNA (e.g., mRNA) vaccine may or may not contain a enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, including (e.g., consisting of) a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In other embodiments the RNA (e.g., mRNA) vaccine may have one or more AU-rich sequences removed. These sequences, sometimes referred to as AURES are destabilizing sequences found in the 3′UTR. The AURES may be removed from the RNA (e.g., mRNA) vaccines. Alternatively the AURES may remain in the RNA (e.g., mRNA) vaccine.

### Polypeptides

It will be appreciated that the polynucleotides of the invention encode a chimeric or fusion protein. The protein encoded by the polynucleotides, eg, mRNA molecules, of the invention may also be termed an “antigenic polypeptide”.

As used herein, “polypeptide” means a polymer of amino acid residues (natural or unnatural) linked together most often by peptide bonds. The term, as used herein, refers to proteins, polypeptides, and peptides of any size, structure, or function. In some instances the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides such as antibodies or insulin and may be associated or linked. Most commonly disulfide linkages are found in multichain polypeptides. The term polypeptide may also apply to amino acid polymers in which one or more amino acid residues are an artificial chemical analogue of a corresponding naturally occurring amino acid.

As used herein, a chimeric or fusion protein refers to a polypeptide that comprises amino acid sequences that are not arranged in the same spatial configuration as occurs in nature. For example, and in the context of the present invention, the chimeric or fusion protein encoded by the polynucleotides of the invention, comprises portions of a full length spike protein, which are in a different spatial arrangement to full length spike protein.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

The present invention contemplates several types of compositions which encode polypeptides, including variants and derivatives. These include substitutional, insertional, deletion and covalent variants and derivatives. The term “derivative” is used synonymously with the term “variant” but generally refers to a molecule that has been modified and/or changed in any way relative to a reference molecule or starting molecule.

As such, mRNAs of the invention encoding polypeptides containing substitutions, insertions and/or additions, deletions and covalent modifications with respect to reference sequences, in particular the polypeptide sequences disclosed herein, are included within the scope of this invention. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more (e.g., 3, 4 or 5) amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides encoded by the polynucleotides include surface manifestations, local conformational shape, folds, loops, half-loops, domains, half-domains, sites, termini and any combination(s) thereof.

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid based embodiments is used synonymously with “amino acid residue” and “amino acid side chain.” As used herein when referring to polynucleotides the terms “site” as it pertains to nucleotide based embodiments is used synonymously with “nucleotide.” A site represents a position within a peptide or polypeptide or polynucleotide that may be modified, manipulated, altered, derivatized or varied within the polypeptide-based or polynucleotide-based molecules.

As used herein the terms “termini” or “terminus” when referring to polypeptides or polynucleotides refers to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N- and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein having a length of 10, 20, 30, 40, 50, 60, 70, 80, 90, 100 or longer than 100 amino acids. In another example, any protein that includes a stretch of 20, 30, 40, 50, or 100 (contiguous) amino acids that are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided herein or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% to 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity,” as known in the art, refers to a relationship between the sequences of two or more polypeptides or polynucleotides, as determined by comparing the sequences. In the art, identity also means the degree of sequence relatedness between two sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al. (1997).” Gapped BLAST and PSI-BLAST: a new generation of protein database search programs,” Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) was developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between nucleic acid molecules (e.g. DNA molecules and/or RNA molecules) and/or between polypeptide molecules. Polymeric molecules (e.g. nucleic acid molecules (e.g. DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

The polypeptides encoded by the polynucleotides of the invention comprise N-terminal signal peptides. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. Signal peptides generally include three regions: an N-terminal region of differing length, which usually comprises positively charged amino acids; a hydrophobic region; and a short carboxy-terminal peptide region. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane. The signal peptide, however, is not responsible for the final destination of the mature protein. Secretory proteins devoid of additional address tags in their sequence are by default secreted to the external environment. During recent years, a more advanced view of signal peptides has evolved, showing that the functions and immunodominance of certain signal peptides are much more versatile than previously anticipated.

In any embodiment, the N-terminal secretion signal peptide may comprise any amino acid sequence which enables the chimeric or fusion protein to be processed by ribosomes bound to the rough endoplasmic reticulum (ER) of a cell, and thereby results in threading of the chimeric or fusion protein into the ER.

Preferably, the N-terminal secretion signal peptide comprises the amino acid sequence of the secretion signal from any coronavirus spike protein, and may not be derived from a spike protein of the same coronavirus strain as the RBD sequence. For example, the RBD sequence may be from a SARS-CoV-2 and the N-terminal secretion signal peptide may be from a SARS virus.

In some embodiments, the signal peptide fused to the antigenic polypeptide is an artificial signal peptide. In some embodiments, an artificial signal peptide fused to the antigenic polypeptide encoded by the RNA (e.g., mRNA) vaccine is obtained from an immunoglobulin protein, e.g., an IgE signal peptide or an IgG signal peptide. In some embodiments, a signal peptide fused to the antigenic polypeptide encoded by a RNA (e.g., mRNA) vaccine is an Ig heavy chain epsilon-1 signal peptide (IgE HC SP) having the sequence of: MDWTWILFLVAAATRVHS. In some embodiments, a signal peptide fused to the antigenic polypeptide encoded by the (e.g., mRNA) RNA (e.g., mRNA) vaccine is an IgGk chain V-Ill region HAH signal peptide (IgGk SP) having the sequence of METPAQLLFLLLLWLPDTTG. In some embodiments, the signal peptide is selected from: Japanese encephalitis PRM signal sequence (MLGSNSGQRVVFTILLLLVAPAYS), VSVg protein signal sequence (MKCLLYLAFLFIGVNCA) and Japanese encephalitis JEV signal sequence (MWLVSLAIVTACAGA).

The examples disclosed herein are not meant to be limiting and any signal peptide that is known in the art to facilitate targeting of a protein to ER for processing and/or targeting of a protein to the cell membrane may be used in accordance with the present disclosure.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

A signal peptide is typically cleaved from the nascent polypeptide at the cleavage junction during ER processing. The mature antigenic polypeptide produce by a respiratory virus RNA (e.g., mRNA) vaccine of the present disclosure typically does not comprise a signal peptide.

In preferred embodiments the invention, the signal peptide comprises the sequence as set forth in SEQ ID NO: 4, or a sequence at least 80%, at least 85%, at least 90%, at least 95%, at least 96%, at least 97%, at least 98% or at least 99% identical thereto.

### Compositions and Lipid Nanoparticles

The present invention contemplates the provision of a polynucleotide encoding an RBD from a coronavirus spike protein, preferably formulated in a lipid nanoparticle. Accordingly, the present invention also provides a lipid nanoparticle comprising a polynucleotide as described herein. It will be appreciated that in any embodiment, the nanoparticles of the invention may also be described as “vaccine” compositions.

In some embodiments, the polynucleotide of the invention (e.g. mRNA) is formulated in a lipid-polycation complex, referred to as a cationic lipid nanoparticle. As a non-limiting example, the polycation may include a cationic peptide or a polypeptide such as, but not limited to, polylysine, polyornithine and/or polyarginine. In some embodiments, the mRNA may be formulated in a lipid nanoparticle that includes a non-cationic lipid such as, but not limited to, cholesterol or dioleoyl phosphatidylethanolamine (DOPE).

In some embodiments, the lipid nanoparticle comprises a cationic lipid, a PEG-modified lipid, a sterol and a non-cationic lipid.

In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (12Z,15Z)-N,N-dimethyl-2-nonylhenicosa-12,15-dien-1-amine (L608), and N,N-dimethyl-1-[(1S,2R)-2-octylcyclopropyl]heptadecan-8-amine (L530). In some embodiments, the lipid is (L608), as described in U.S. Pat. No. 10,702,600B1.

In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

A lipid nanoparticle formulation may be influenced by, but not limited to, the selection of the cationic lipid component, the degree of cationic lipid saturation, the nature of the PEGylation, ratio of all components and biophysical parameters such as size. In one example by Semple et al. (Nature Biotech. 2010 28:172-176), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (Basha et al. Mol Ther. 2011 19:2186-2200).

In some embodiments, lipid nanoparticle formulations may comprise 35 to 45% cationic lipid, 40% to 50% cationic lipid, 50% to 60% cationic lipid and/or 55% to 65% cationic lipid. In some embodiments, the ratio of lipid to RNA (e.g., mRNA) in lipid nanoparticles may be 5:1 to 20:1, 10:1 to 25:1, 15:1 to 30:1 and/or at least 30:1.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 6.0% of the lipid molar ratio of PEG-c-DOMG (R-3-[(w-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, a polynucleotide (e.g. mRNA) vaccine formulation of the invention is a nanoparticle that comprises at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids.

The amino alcohol cationic lipid may be the lipids described in and/or made by the methods described in U.S. Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z, 12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, a lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of 20-60% cationic lipid: 5-25% neutral lipid: 25-55% sterol; 0.5-15% PEG-lipid.

In some embodiments, a lipid nanoparticle formulation includes 25% to 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., 35 to 65%, 45 to 65%, 60%, 57.5%, 50% or 40% on a molar basis.

In some embodiments, a lipid nanoparticle formulation includes 0.5% to 15% on a molar basis of the neutral lipid, e.g., 3 to 12%, 5 to 10% or 15%, 10%, or 7.5% on a molar basis. Examples of neutral lipids include, without limitation, DSPC, POPC, DPPC, DOPE and SM. In some embodiments, the formulation includes 5% to 50% on a molar basis of the sterol (e.g., 15 to 45%, 20 to 40%, 40%, 38.5%, 35%, or 31% on a molar basis. A non-limiting example of a sterol is cholesterol. In some embodiments, a lipid nanoparticle formulation includes 0.5% to 20% on a molar basis of the PEG or PEG-modified lipid (e.g., 0.5 to 10%, 0.5 to 5%, 1.5%, 0.5%, 1.5%, 3.5%, or 5% on a molar basis. In some embodiments, a PEG or PEG modified lipid comprises a PEG molecule of an average molecular weight of 2,000 Da. In some embodiments, a PEG or PEG modified lipid comprises a PEG molecule of an average molecular weight of less than 2,000, for example around 1,500 Da, around 1,000 Da, or around 500 Da. Non-limiting examples of PEG-modified lipids include PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in their entirety).

In some embodiments, lipid nanoparticle formulations include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In some embodiments, lipid nanoparticle formulations include 35-65% of a cationic lipid selected from 2,2-dilinoleyl-4-dimethylaminoethyl-[11,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In some embodiments, lipid nanoparticle formulations include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In some embodiments, lipid nanoparticle formulations include 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 7.5% of the neutral lipid, 31% of the sterol, and 1.5% of the PEG or PEG-modified lipid on a molar basis.

In some embodiments, lipid nanoparticle formulations include 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 10% of the neutral lipid, 38.5% of the sterol, and 1.5% of the PEG or PEG-modified lipid on a molar basis.

In some embodiments, lipid nanoparticle formulations include 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 10% of the neutral lipid, 35% of the sterol, 4.5% or 5% of the PEG or PEG-modified lipid, and 0.5% of the targeting lipid on a molar basis.

In some embodiments, lipid nanoparticle formulations include 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 15% of the neutral lipid, 40% of the sterol, and 5% of the PEG or PEG-modified lipid on a molar basis.

In some embodiments, lipid nanoparticle formulations include 57.2% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 7.1% of the neutral lipid, 34.3% of the sterol, and 1.4% of the PEG or PEG-modified lipid on a molar basis.

In some embodiments, lipid nanoparticle formulations include 57.5% of a cationic lipid selected from the PEG lipid is PEG-cDMA (PEG-cDMA is further discussed in Reyes et al. (J. Controlled Release, 107, 276-287 (2005), the contents of which are herein incorporated by reference in their entirety), 7.5% of the neutral lipid, 31.5% of the sterol, and 3.5% of the PEG or PEG-modified lipid on a molar basis.

In some embodiments, lipid nanoparticle formulations consists essentially of a lipid mixture in molar ratios of 20-70% cationic lipid: 5-45% neutral lipid: 20-55% cholesterol: 0.5-15% PEG-modified lipid. In some embodiments, lipid nanoparticle formulations consists essentially of a lipid mixture in a molar ratio of 20-60% cationic lipid: 5-25% neutral lipid: 25-55% cholesterol: 0.5-15% PEG-modified lipid.

In some embodiments, the molar lipid ratio is 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In some embodiments, lipid nanoparticle formulations may comprise a cationic lipid, a PEG lipid and a structural lipid and optionally comprise a non-cationic lipid. As a non-limiting example, a lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, a lipid nanoparticle may comprise 55% cationic lipid, 10% non-cationic lipid, 2.5% PEG lipid and 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise 40-60% of cationic lipid, 5-15% of a non-cationic lipid, 1-2% of a PEG lipid and 30-50% of a structural lipid. As another non-limiting example, the lipid nanoparticle may comprise 50% cationic lipid, 10% non-cationic lipid, 1.5% PEG lipid and 38.5% structural lipid. As yet another non-limiting example, the lipid nanoparticle may comprise 55% cationic lipid, 10% non-cationic lipid, 2.5% PEG lipid and 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may comprise a cationic lipid, a non-cationic lipid, a PEG lipid and a structural lipid. As a non-limiting example, the lipid nanoparticle comprise 50% of the cationic lipid DLin-KC2-DMA, 10% of the non-cationic lipid DSPC, 1.5% of the PEG lipid PEG-DOMG and 38.5% of the structural lipid cholesterol. As a non-limiting example, the lipid nanoparticle comprise 50% of the cationic lipid DLin-MC3-DMA, 10% of the non-cationic lipid DSPC, 1.5% of the PEG lipid PEG-DOMG and 38.5% of the structural lipid cholesterol. As a non-limiting example, the lipid nanoparticle comprise 50% of the cationic lipid DLin-MC3-DMA, 10% of the non-cationic lipid DSPC, 1.5% of the PEG lipid PEG-DMG and 38.5% of the structural lipid cholesterol. As yet another non-limiting example, the lipid nanoparticle comprise 55% of the cationic lipid L319, 10% of the non-cationic lipid DSPC, 2.5% of the PEG lipid PEG-DMG and 32.5% of the structural lipid cholesterol.

Relative amounts of the active ingredient, the pharmaceutically acceptable excipient, and/or any additional ingredients in a vaccine composition may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the polynucleotide (e.g. mRNA) vaccine composition of the invention may comprise the polynucleotide described herein, formulated in a lipid nanoparticle comprising MC3 (DLin-MC3-DMA), Cholesterol, DSPC and PEG2000-DMG, the buffer trisodium citrate, sucrose and water for injection. As a non-limiting example, the composition comprises: 2.0 mg/mL of drug substance (e.g., polynucleotides encoding H10N8 hMPV), 21.8 mg/mL of MC3, 10.1 mg/mL of cholesterol, 5.4 mg/mL of DSPC, 2.7 mg/mL of PEG2000-DMG, 5.16 mg/mL of trisodium citrate, 71 mg/mL of sucrose and 1.0 mL of water for injection.

In some embodiments, a nanoparticle (e.g., a lipid nanoparticle) has a mean diameter of 10-500 nm, 20-400 nm, 30-300 nm, 40-200 nm. In some embodiments, a nanoparticle (e.g., a lipid nanoparticle) has a mean diameter of 50-150 nm, 50-200 nm, 80-100 nm or 80-200 nm.

In some embodiments the RNA (e.g., mRNA) vaccine may be associated with a cationic or polycationic compounds, including protamine, nucleoline, spermine or spermidine, or other cationic peptides or proteins, such as poly-L-lysine (PLL), polyarginine, basic polypeptides, cell penetrating peptides (CPPs), including HIV-binding peptides, HIV-1 Tat (HIV), Tat-derived peptides, Penetratin, VP²² derived or analog peptides, Pestivirus Erns, HSV, VP²² (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, plsl, FGF, Lactoferrin, Transportan, Buforin-2, Bac715-24, SynB, SynB(1), pVEC, hCT-derived peptides, SAP, histones, cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio)propane, DC-6-14: O,O-ditetradecanoyl-N-.alpha.-trimethylammonioacetyl)diethanolamine chloride, CLIP 1: rac-[(2,3-dioctadecyloxypropyl)(2-hydroxyethyl)]-dimethylammonium chloride, CLIP6: rac-[2(2,3-dihexadecyloxypropyloxymethyloxy)ethyl]-trimethylammonium, CLIP9: rac-[2(2,3-dihexadecyloxypropyloxysuccinyloxy)ethyl]-trimethylammonium, oligofectamine, or cationic or polycationic polymers, e.g. modified polyaminoacids, such as beta-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole), etc.

In other embodiments the RNA (e.g., mRNA) vaccine is not associated with a cationic or polycationic compounds.

Other examples of suitable lipid nanoparticle formulations are provided in U.S. Pat. No. 10,702,600, the contents of which are hereby incorporated by reference.

### Liposomes, Lipoplexes, and Lipid Nanoparticles

The RNA (e.g., mRNA) vaccines of the disclosure can be formulated using one or more liposomes, lipoplexes, or lipid nanoparticles. In some embodiments, pharmaceutical compositions of RNA (e.g., mRNA) vaccines include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In some embodiments, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, liposome formulations may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In some embodiments, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

In some embodiments, the RNA (e.g., mRNA) vaccine pharmaceutical compositions may be formulated in liposomes such as, but not limited to, DiLa2 liposomes (Marina Biotech, Bothell, Wash.), SMARTICLES@(Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In some embodiments, the cationic lipid may be a low molecular weight cationic lipid such as those described in U.S. Patent Application No. 20130090372, the contents of which are herein incorporated by reference in their entirety.

In some embodiments, the RNA (e.g., mRNA) vaccines may be formulated in a lipid vesicle, which may have crosslinks between functionalized lipid bilayers.

In some embodiments, the RNA (e.g., mRNA) vaccines may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the RNA (e.g., mRNA) vaccines may be formulated in a lipid-polycation complex, which may further include a non-cationic lipid such as, but not limited to, cholesterol or dioleoyl phosphatidylethanolamine (DOPE).

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG (R-3-[(w-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, the RNA (e.g., mRNA) vaccines may be formulated in a lipid nanoparticle.

In some embodiments, the RNA (e.g., mRNA) vaccine formulation comprising the polynucleotide is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another embodiment, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in U.S. Patent Publication No. US20130150625, herein incorporated by reference in its entirety. As a non-limiting example, the cationic lipid may be 2-amino-3-[(9Z, 12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z, 12Z)-octadeca-9,12-dien-1-yloxy]-2-[(octyloxy)methyl]propan-1-ol (Compound 3 in US20130150625); and 2-(dimethylamino)-3-[(9Z, 12Z)-octadeca-9,12-dien-1-yloxy]-2-{[(9Z, 12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, the lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid: 5-25% neutral lipid: 25-55% sterol; 0.5-15% PEG-lipid.

In some embodiments, the formulation includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In some embodiments, the formulation includes from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In some embodiments, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), PEG-cDMA (further discussed in Reyes et al. J. Controlled Release, 107, 276-287 (2005) the contents of which are herein incorporated by reference in their entirety)

In some embodiments, the formulations of the present disclosure include 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In some embodiments, the formulations of the present disclosure include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In some embodiments, the formulations of the present disclosure include 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In some embodiments, the formulations of the present disclosure include about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In some embodiments, the formulations of the present disclosure include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In some embodiments, the formulations of the present disclosure include about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In some embodiments, the formulations of the present disclosure include about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In some embodiments, the formulations of the present disclosure include about 57.2% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.1% of the neutral lipid, about 34.3% of the sterol, and about 1.4% of the PEG or PEG-modified lipid on a molar basis.

In some embodiments, the formulations of the present disclosure include about 57.5% of a cationic lipid selected from the PEG lipid is PEG-cDMA (PEG-cDMA is further discussed in Reyes et al. (J. Controlled Release, 107, 276-287 (2005), the contents of which are herein incorporated by reference in their entirety), about 7.5% of the neutral lipid, about 31.5% of the sterol, and about 3.5% of the PEG or PEG-modified lipid on a molar basis.

In some embodiments, lipid nanoparticle formulation consists essentially of a lipid mixture in molar ratios of about 20-70% cationic lipid: 5-45% neutral lipid: 20-55% cholesterol: 0.5-15% PEG-modified lipid; more preferably in a molar ratio of about 20-60% cationic lipid: 5-25% neutral lipid: 25-55% cholesterol: 0.5-15% PEG-modified lipid.

In some embodiments, the molar lipid ratio is approximately 50/10/38.5/1.5 (mol % cationic lipid/neutral lipid, e.g., DSPC/Chol/PEG-modified lipid, e.g., PEG-DMG, PEG-DSG or PEG-DPG), 57.2/7.1134.3/1.4 (mol % cationic lipid/neutral lipid, e.g., DPPC/Chol/PEG-modified lipid, e.g., PEG-cDMA), 40/15/40/5 (mol % cationic lipid/neutral lipid, e.g., DSPC/Chol/PEG-modified lipid, e.g., PEG-DMG), 50/10/35/4.5/0.5 (mol % cationic lipid/neutral lipid, e.g., DSPC/Chol/PEG-modified lipid, e.g., PEG-DSG), 50/10/35/5 (cationic lipid/neutral lipid, e.g., DSPC/Chol/PEG-modified lipid, e.g., PEG-DMG), 40/10/40/10 (mol % cationic lipid/neutral lipid, e.g., DSPC/Chol/PEG-modified lipid, e.g., PEG-DMG or PEG-cDMA), 35/15/40/10 (mol % cationic lipid/neutral lipid, e.g., DSPC/Chol/PEG-modified lipid, e.g., PEG-DMG or PEG-cDMA) or 52/13/30/5 (mol % cationic lipid/neutral lipid, e.g., DSPC/Chol/PEG-modified lipid, e.g., PEG-DMG or PEG-cDMA).

In a particularly preferred embodiment, the lipid nanoparticle comprises or consist of
a cationic and/or ionisable lipid comprising from about 40 mol % to about 60 mol % of the total lipid present in the nanoparticle;
a phospholipid comprising from about 5 mol % to about 20 mol % of the total lipid present in the nanoparticle;
a structural lipid comprising from about 30 mol % to about 50 mol % of the total lipid present in the nanoparticle;
a PEGylated lipid comprising from about 0.05 mol % to less than 0.5 mol % of the total lipid present in the nanoparticle.

In this embodiment, the PEGylated lipid may comprise from about from about 0.06 mol % to about 0.5 mol %, from about 0.07 mol % to about 0.5 mol %, from about 0.08 mol % to about 0.5 mol %, from about 0.09 mol % to about 0.5 mol %, from about 0.1 mol % to about 0.5 mol %, from about 0.15 mol % to about 0.5 mol %, from about 0.2 mol % to about 0.5 mol %, from about 0.25 mol % to about 0.5 mol %, from about 0.3 mol % to about 0.5 mol %, from about 0.3 mol % to about 0.5 mol %, from about 0.35 mol % to about 0.5 mol %, from about 0.4 mol % to about 0.5 mol %, from about 0.45 mol % to about 0.5 mol %, from about 0.05 mol % to about 0.45 mol %, from about 0.05 mol % to about 0.4 mol %, from about 0.05 mol % to about 0.35 mol %, from about 0.05 mol % to about 0.3 mol %, from about 0.05 mol % to about 0.25 mol %, from about 0.05 mol % to about 0.2 mol %, from about 0.05 mol % to about 0.15 mol %, from about 0.05 mol % to about 0.1 mol %, from about 0.05 mol % to about 0.09 mol %, from about 0.05 mol % to about 0.08 mol %, from about 0.05 mol % to about 0.07 mol %, or from about 0.05 mol % to about 0.06 mol % of the total lipid present in the particle.

In this embodiment, the PEGylated lipid may comprise from 0.06 mol % to 0.5 mol %, from 0.07 mol % to 0.5 mol %, from 0.08 mol % to 0.5 mol %, from 0.09 mol % to 0.5 mol %, from 0.1 mol % to 0.5 mol %, from 0.15 mol % to 0.5 mol %, from 0.2 mol % to 0.5 mol %, from 0.25 mol % to 0.5 mol %, from 0.3 mol % to 0.5 mol %, from 0.3 mol % to 0.5 mol %, from 0.35 mol % to 0.5 mol %, from 0.4 mol % to 0.5 mol %, from 0.45 mol % to 0.5 mol %, from 0.05 mol % to 0.45 mol %, from 0.05 mol % to 0.4 mol %, from 0.05 mol % to 0.35 mol %, from 0.05 mol % to 0.3 mol %, from 0.05 mol % to 0.25 mol %, from 0.05 mol % to 0.2 mol %, from 0.05 mol % to 0.15 mol %, from 0.05 mol % to 0.1 mol %, from 0.05 mol % to 0.09 mol %, from 0.05 mol % to 0.08 mol %, from 0.05 mol % to 0.07 mol %, or from 0.05 mol % to 0.06 mol % of the total lipid present in the particle.

In this embodiment, the PEGylated lipid may comprise 0.05 mol %, 0.06 mol %, 0.07 mol %, 0.08 mol %, 0.09 mol %, 0.1 mol %, 0.15 mol %, 0.2 mol %, 0.25 mol %, 0.3 mol %, 0.35 mol %, 0.4 mol %, 0.45 mol % of the total lipid present in the particle.

In this embodiment, the PEGylated lipid may be selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof.

In this embodiment, the PEGylated lipid may be selected from the group consisting of PEG-c-DOMG, PEG-DMG, PEG-DLPE, PEG-DMPE, PEG-DPPC, or a PEG-DSPE lipid.

In this embodiment, the PEGylated lipid may be PEG-DSPE.

In this embodiment, the PEGylated lipid may have a PEG component that has a molecular weight between about 100 Da and about 100,000 Dam between about 100 Da and about 100,000 Da, between about 1000 Da and 9,000 Da, between about 1000 Da and 8,000 Da, between about 1000 Da and 7,000 Da, between about 1000 Da and 6,000 Da, between about 1000 Da and 5,000 Da, between about 1000 Da and 4,000 Da, between about 1000 Da and 3,000 Da, or between about 1000 Da and 2,000 Da.

In this embodiment, the PEGylated lipid may have a PEG component that has a molecular weight of between about 1,000 Da and 5,000 Da, preferably between about 2,000 Da and 5,000 Da.

In this embodiment, the PEGylated lipid may have a PEG component that has a molecular weight of 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da.

In this embodiment, the PEGylated lipid may be DSPE-PEG, wherein the PEG has a molecular weight of 2000 Da.

In this embodiment, the cationic lipid may be any one of N,N-dioleyl-N,N-dimethylammonium chloride (DODAC), 1,2-dioeoyloxy-3-(dimethylamino)propane (DODAP), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3.beta.-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 4-Hydroxybutyl)azanediyl)bis(hexane-6,1-diyl) bis(2-hexyldecanoate) (ALC-0315), 8-[(2-hydroxyethyl)[6-oxo-6-(undecyloxy)hexyl]amino]-octanoic acid,1-octylnonyl ester (SM-102) and mixtures thereof.

In this embodiment, the cationic lipid may be of Formula I

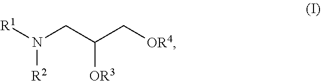

wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation, preferably the cationic lipid of Formula I is 1,2-dilinoleyloxy-N,N-dimethylaminopropane (DLinDMA) or 1,2-dilinolenyloxy-N,N-dimethylaminopropane (DLenDMA).

In this embodiment, the cationic lipid may be of Formula II

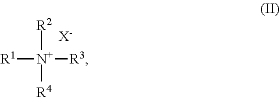

wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation.

In this embodiment, the cationic lipid may be of Formula III

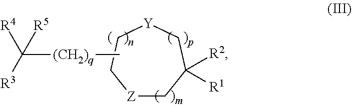

wherein R¹ and R² are either the same or different and independently optionally substituted C₁₂-C₂₄ alkyl, optionally substituted C₁₂-C₂₄ alkenyl, optionally substituted C₁₂-C₂₄ alkynyl, or optionally substituted C₁₂-C₂₄ acyl; R³ and R⁴ are either the same or different and independently optionally substituted C₁-C₆ alkyl, optionally substituted C₁-C₆ alkenyl, or optionally substituted C₁-C₅ alkynyl or R³ and R⁴ may join to form an optionally substituted heterocyclic ring of 4 to 6 carbon atoms and 1 or 2 heteroatoms chosen from nitrogen and oxygen; R⁵ is either absent or hydrogen or C1-C6 alkyl to provide a quaternary amine; m, n, and p are either the same or different and independently either 0 or 1 with the proviso that m, n, and p are not simultaneously 0; q is 0, 1, 2, 3, or 4; and Y and Z are either the same or different and independently O, S, or NH.

In this embodiment, the cationic lipid of Formula III may be 2,2-dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), or mixtures thereof.

In this embodiment, the cationic lipid may be DODAP, DLin-DMA, DLin-K-DMA, DLin-K2-DMA DLin-MC3-DMA.

In this embodiment, the cationic lipid may comprise from about 40 mol % to about 60 mol %, from about 40 mol % to about 55 mol %, from about 40 mol % to about 50 mol %, from about 40 mol % to about 45 mol %, from about 45 mol % to about 60 mol %, from about 50 mol % to about 60 mol %, or from about 55 mol % to about 60 mol % of the total lipid present in the particle.

In this embodiment, the cationic lipid may comprise about 40, 41, 42, 43, 44, 45, 46, 47, 48, 49, 50, 51, 52, 53, 54, 55, 56, 57, 58, 59 or 60 mol % of the total lipid present in the particle.

In this embodiment, the phospholipid may be a cationic phospholipid, an unsaturated lipid, or a polyunsaturated lipid.

In this embodiment, the phospholipid is according to Formula (IV):

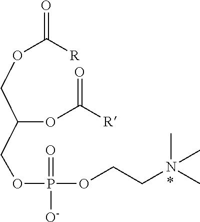

in which represents a phospholipid moiety and R and R′ represent fatty acid moieties with or without unsaturation that may be the same or different.

In this embodiment, the phospholipid moiety may be selected from the group consisting of: phosphatidyl choline, phosphatidyl ethanolamine, phosphatidyl glycerol, phosphatidyl serine, phosphatidic acid, 2-lysophosphatidyl choline, and a sphingomyelin.

In this embodiment, the phospholipid may have a fatty acid moiety selected from the non-limiting group consisting of: lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

In this embodiment, the phosopholipid may be lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof.

In this embodiment, the phosopholipid may be distearoylphosphatidylcholine (DSPC).

In this embodiment, the phospholipid may comprise from about 5 mol % to about 20 mol %, from about 5 mol % to about 15 mol %, from about 5 mol % to about 10 mol %, from about 10 mol % to about 20 mol %, from about 15 mol % to about 20 mol % of the total lipid present in the particle.

In this embodiment, the phospholipid may comprise from 5 mol % to 20 mol %, from 5 mol % to 15 mol %, from 5 mol % to 10 mol %, from 10 mol % to 20 mol %, or from 15 mol % to 20 mol % of the total lipid present in the particle.

In this embodiment, the structural lipid may be selected from the group consisting of cholesterol, fecosterol, sitosterol, ergosterol, campesterol, stigmasterol, brassicasterol, tomatidine, tomatine, ursolic acid, alpha-tocopherol, and mixtures thereof.

In this embodiment, the structural lipid may be cholesterol. In some embodiments, the structural lipid includes cholesterol and a corticosteroid (such as prednisolone, dexamethasone, prednisone, and hydrocortisone), or a combination thereof. Further, the structural lipid may be squalene, squalene or combination thereof.

The structural lipid may include lipids containing geranyl acetate, farnesyl acetate or geranyl-geranyl, or ether, ester, or other derivatives.

In this embodiment, the structural lipid may comprise from about 30 mol % to about 50 mol %, from about 30 mol % to about 45 mol %, from about 30 mol % to about 40 mol %, from about 30 mol % to about 35 mol %, from about 35 mol % to about 50 mol %, from about 40 mol % to about 50 mol %, or from about 45 mol % to about 50 mol % of the total lipid present in the particle.

In this embodiment, the structural lipid may comprise from 30 mol % to 50 mol %, from 30 mol % to 45 mol %, from 30 mol % to 40 mol %, from 30 mol % to 35 mol %, from 35 mol % to 50 mol %, from 40 mol % to 50 mol %, or from 45 mol % to 50 mol % of the total lipid present in the particle.

Examples of lipid nanoparticle compositions and methods of making same are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

In some embodiments, the lipid nanoparticle formulations described herein may comprise a cationic lipid, a PEG lipid and a structural lipid and optionally comprise a non-cationic lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may be 4 component lipid nanoparticles. The lipid nanoparticle may comprise a cationic lipid, a non-cationic lipid, a PEG lipid and a structural lipid. As a non-limiting example, the lipid nanoparticle may comprise about 40-60% of cationic lipid, about 5-15% of a non-cationic lipid, about 1-2% of a PEG lipid and about 30-50% of a structural lipid. As another non-limiting example, the lipid nanoparticle may comprise about 50% cationic lipid, about 10% non-cationic lipid, about 1.5% PEG lipid and about 38.5% structural lipid. As yet another non-limiting example, the lipid nanoparticle may comprise about 55% cationic lipid, about 10% non-cationic lipid, about 2.5% PEG lipid and about 32.5% structural lipid. In some embodiments, the cationic lipid may be any cationic lipid described herein such as, but not limited to, DLin-KC2-DMA, DLin-MC3-DMA and L319.

In some embodiments, the lipid nanoparticle formulations described herein may comprise a cationic lipid, a non-cationic lipid, a PEG lipid and a structural lipid. As a non-limiting example, the lipid nanoparticle comprise about 50% of the cationic lipid DLin-KC2-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DOMG and about 38.5% of the structural lipid cholesterol. As a non-limiting example, the lipid nanoparticle comprise about 50% of the cationic lipid DLin-MC3-DMA, about 10% of the non-cationic lipid DSPC, about 1.5% of the PEG lipid PEG-DMG and about 38.5% of the structural lipid cholesterol. As yet another non-limiting example, the lipid nanoparticle comprise about 55% of the cationic lipid L319, about 10% of the non-cationic lipid DSPC, about 2.5% of the PEG lipid PEG-DMG and about 32.5% of the structural lipid cholesterol.

As a non-limiting example, the cationic lipid may be selected from (20Z,23Z)-N,N-dimethylnonacosa-20,23-dien-10-amine, (17Z,20Z)-N,N-dimemylhexacosa-17,20-dien-9-amine, (1Z,19Z)-N5N-dimethylpentacosa-16, 19-dien-8-amine, (13Z,16Z)-N,N-dimethyldocosa-13,16-dien-5-amine, (12Z, 15Z)-N,N-dimethylhenicosa-12,15-dien-4-amine, (14Z, 17Z)-N,N-dimethyltricosa-14,17-dien-6-amine, (15Z, 18Z)-N,N-dimethyltetracosa-15,18-dien-7-amine, (18Z,21Z)-N,N-dimethylheptacosa-18,21-dien-10-amine, (15Z, 18Z)-N,N-dimethyltetracosa-15,18-dien-5-amine, (14Z, 17Z)-N,N-dimethyltricosa-14,17-dien-4-amine, (19Z,22Z)-N,N-dimeihyloctacosa-19,22-dien-9-amine, (18Z,21 Z)-N,N-dimethylheptacosa-18,21-dien-8-amine, (17Z,20Z)-N,N-dimethylhexacosa-17,20-dien-7-amine, (16Z, 19Z)-N,N-dimethylpentacosa-16,19-dien-6-amine, (22Z,25Z)-N,N-dimethylhentriaconta-22,25-dien-10-amine, (21 Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z, 15Z)-N,N-dimethyl-2-nonylhenicosa-12,15-dien-1-amine, (13Z, 16Z)-N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1 R,2S)-2-undecylcyclopropyl]tetradecan-5-amine, N,N-dimethyl-3-{7-[(1S,2R)-2-octylcyclopropyl]heptyl}dodecan-1-amine, 1-[(1 R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z, 12Z)-octadeca-9,12-dien-1-yloxy]-3-(octyloxy)propan-2-amine, S—N,N-dimethyl-1-[(9Z, 12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z, 12Z)-octadeca-9,12-dien-1-yloxy]-3-[(5Z)-oct-5-en-1-yloxy]propan-2-amine, 1-{2-[(9Z, 12Z)-octadeca-9,12-dien-1-yloxy]-1-[(octyloxy)methyl]ethyl}azetidine, (2S)-1-(hexyloxy)-N,N-dimethyl-3-[(9Z, 12Z)-octadeca-9,12-dien-1-yloxy]propan-2-amine, (2S)-1-(heptyloxy)-N,N-dimethyl-3-[(9Z, 12Z)-octadeca-9,12-dien-1-yloxy]propan-2-amine, N,N-dimethyl-1-(nonyloxy)-3-[(9Z, 12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z, 12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z, 16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z, 12Z)-octadeca-9,12-dien-1-yloxy]propan-2-amine, (2R)-1-[(3,7-dimethyloctyl)oxy]-N,N-dimethyl-3-[(9Z, 12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oc1ylcyclopropyl)octyl]oxy}-3-(octyloxy)propan-2-amine and (11E,20Z,23Z)-N,N-dimethylnonacosa-11,20,2-trien-10-amine or a pharmaceutically acceptable salt or stereoisomer thereof.

In some embodiments, the LNP formulations of the RNA (e.g., mRNA) vaccines may contain PEG-c-DOMG at 3% lipid molar ratio. In some embodiments, the LNP formulations of the RNA (e.g., mRNA) vaccines may contain PEG-c-DOMG at 1.5% lipid molar ratio.

In some embodiments, the pharmaceutical compositions of the RNA (e.g., mRNA) vaccines may include at least one of the PEGylated lipids described in International Publication No. WO2012099755, the contents of which are herein incorporated by reference in their entirety.

In some embodiments, the LNP formulation may contain PEG-DMG 2000 (1,2-dimyristoyl-sn-glycero-3-phophoethanolamine-N-[methoxy(polyethylene glycol)-2000). In some embodiments, the LNP formulation may contain PEG-DMG 2000, a cationic lipid known in the art and at least one other component. In some embodiments, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA (e.g., mRNA) vaccines, PNAS 2012; PMID: 22908294, the contents of each of which are herein incorporated by reference in their entirety).In certain preferred embodiments, the LNP formulation may comprise between about 20% to 60% ionizable cationic lipid (preferably between about 30 to 50% or 35 to 40%), between about 5% to 25% neutral lipid (preferably between about 10% to about 20%), between about 25% to 55% cholesterol (preferably between about 30% to about 50% or about 35% to about 45%), and/or between about 0.5% to about 15% PEG-modified lipid (preferably between about 1% to about 12% or between about 2% to about 10%).

In particularly preferred embodiments, the ionisable lipid comprises DLin-MC3-DMA [(6Z,9Z,28Z,31Z)-heptatriaconta-6,9,28,31 tetraen-19-yl-4-(dimethylamino) butanoate] or ALC-0315; the PEGylated lipid comprises Polyethylene glycol [PEG]2000 dimyristoyl glycerol; and the structural lipid comprises one or both of cholesterol and distearoylphophatidylcholine.

Preferably the LNP formulation may comprise any one of the formulations described herein in any of the Examples.

In preferred embodiments, the ionisable lipid is DLin-MC3-DMA [(6Z,9Z,28Z,31Z)-heptatriaconta-6,9,28,31 tetraen-19-yl-4-(dimethylamino) butanoate] or ALC-0315 and is present in the formulation in an amount of about 40% to about 50% (lipid ratio); the PEGylated lipid is Polyethylene glycol [PEG]2000 dimyristoyl glycerol and is present in the formulation in an amount of about 0.1% to about 2%; optionally between about 0.2% and 1.6% (lipid ratio); and the structural lipid comprises one or both of cholesterol and distearoylphophatidylcholine, wherein the cholesterol is present in the formulation in an amount of between about 35% to about 45% (lipid ratio), preferably between about 37% to about 44%, more preferably between about 39% to about 43%; and/or wherein the distearoylphophatidylcholine (DSPC) is present in an amount of between about 5% to about 15%, preferably about 10%.

In preferred embodiments, the ionisable lipid is DLin-MC3-DMA [(6Z,9Z,28Z,31Z)-heptatriaconta-6,9,28,31 tetraen-19-yl-4-(dimethylamino) butanoate] or ALC-0315 and is present in the formulation in an amount of about 40% to about 50% (mol); the PEGylated lipid is Polyethylene glycol [PEG]2000 dimyristoyl glycerol and is present in the formulation in an amount of about 0.1% to about 2%; optionally between about 0.2% and 1.6% (mol); and the structural lipid comprises one or both of cholesterol and distearoylphophatidylcholine, wherein the cholesterol is present in the formulation in an amount of between about 35% to about 45% (mol), preferably between about 37% to about 44%, more preferably between about 39% to about 43%; and/or wherein the distearoylphophatidylcholine (DSPC) is present in an amount of between about 5% to about 15%, preferably about 10% mol.

In preferred embodiments, the LNP formulation comprises 4 lipids DLin-MC3-DMA, Cholesterol, DSPC and DMG-PEG 2000 at a ratio of 50:39.8:10:0.2 (mol ratio).

The lipid nanoparticles described herein may be made in a sterile environment.

In some embodiments, the LNP formulation may be formulated in a nanoparticle such as a nucleic acid-lipid particle. As a non-limiting example, the lipid particle may comprise one or more active agents or therapeutic agents; one or more cationic lipids comprising from about 50 mol % to about 85 mol % of the total lipid present in the particle; one or more non-cationic lipids comprising from about 13 mol % to about 49.5 mol % of the total lipid present in the particle; and one or more conjugated lipids that inhibit aggregation of particles comprising from about 0.5 mol % to about 2 mol % of the total lipid present in the particle.

The nanoparticle formulations may comprise a phosphate conjugate. The phosphate conjugate may increase in vivo circulation times and/or increase the targeted delivery of the nanoparticle. As a non-limiting example, the phosphate conjugates may include a compound of any one of the formulas described in International Application No. WO2013033438, the contents of which are herein incorporated by reference in its entirety.

The nanoparticle formulation may comprise a polymer conjugate. The polymer conjugate may be a water soluble conjugate. The polymer conjugate may have a structure as described in U.S. Patent Application No. 20130059360, the contents of which are herein incorporated by reference in its entirety. In some embodiments, polymer conjugates with the polynucleotides of the present disclosure may be made using the methods and/or segmented polymeric reagents described in U.S. Patent Application No. 20130072709, the contents of which are herein incorporated by reference in its entirety. In some embodiments, the polymer conjugate may have pendant side groups comprising ring moieties such as, but not limited to, the polymer conjugates described in U.S. Patent Publication No. US20130196948, the contents which are herein incorporated by reference in its entirety.

The nanoparticle formulations may comprise a conjugate to enhance the delivery of nanoparticles of the present disclosure in a subject. Further, the conjugate may inhibit phagocytic clearance of the nanoparticles in a subject. In one embodiment, the conjugate may be a “self” peptide designed from the human membrane protein CD47 (e.g., the “self” particles described by Rodriguez et al. (Science 2013 339, 971-975), herein incorporated by reference in its entirety). As shown by Rodriguez et al., the self peptides delayed macrophage-mediated clearance of nanoparticles which enhanced delivery of the nanoparticles. In another embodiment, the conjugate may be the membrane protein CD47 (e.g., see Rodriguez et al. Science 2013 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In some embodiments, the RNA (e.g., mRNA) vaccines of the present disclosure are formulated in nanoparticles which comprise a conjugate to enhance the delivery of the nanoparticles of the present disclosure in a subject. The conjugate may be the CD47 membrane or the conjugate may be derived from the CD47 membrane protein, such as the “self” peptide described previously. In some embodiments, the nanoparticle may comprise PEG and a conjugate of CD47 or a derivative thereof. In some embodiments, the nanoparticle may comprise both the “self” peptide described above and the membrane protein CD47.

In some embodiments, a “self” peptide and/or CD47 protein may be conjugated to a virus-like particle or pseudovirion, as described herein for delivery of the RNA (e.g., mRNA) vaccines of the present disclosure.

In some embodiments, RNA (e.g., mRNA) vaccine pharmaceutical compositions comprising the polynucleotides of the present disclosure and a conjugate that may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in U.S. Patent Publication No. US20130184443, the contents of which are herein incorporated by reference in their entirety.

The nanoparticle formulations may be a carbohydrate nanoparticle comprising a carbohydrate carrier and a RNA (e.g., mRNA) vaccine. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; the contents of which are herein incorporated by reference in their entirety).

Nanoparticle formulations of the present disclosure may be coated with a surfactant or polymer in order to improve the delivery of the particle. In some embodiments, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, RNA (e.g., mRNA) vaccines within the central nervous system. As a non-limiting example nanoparticles comprising a hydrophilic coating and methods of making such nanoparticles are described in U.S. Patent Publication No. US20130183244, the contents of which are herein incorporated by reference in their entirety.

In some embodiments, the lipid nanoparticles of the present disclosure may be hydrophilic polymer particles. Non-limiting examples of hydrophilic polymer particles and methods of making hydrophilic polymer particles are described in U.S. Patent Publication No. US20130210991, the contents of which are herein incorporated by reference in their entirety.

In some embodiments, the lipid nanoparticles of the present disclosure may be hydrophobic polymer particles.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In some embodiments, the internal ester linkage may be located on either side of the saturated carbon.

In some embodiments, an immune response may be elicited by delivering a lipid nanoparticle which may include a nanospecies, a polymer and an immunogen. (U.S. Publication No. 20120189700 and International Publication No. WO2012099805; each of which is herein incorporated by reference in their entirety). The polymer may encapsulate the nanospecies or partially encapsulate the nanospecies. The immunogen may be a recombinant protein, a modified RNA and/or a polynucleotide described herein. In some embodiments, the lipid nanoparticle may be formulated for use in a vaccine such as, but not limited to, against a pathogen.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosa tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028, the contents of each of which are herein incorporated by reference in its entirety.

The lipid nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. Non-limiting examples of biocompatible polymers are described in International Patent Publication No. WO2013116804, the contents of which are herein incorporated by reference in their entirety. The polymeric material may additionally be irradiated. As a non-limiting example, the polymeric material may be gamma irradiated (see e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a co-polymer such as, but not limited to, a block co-polymer (such as a branched polyether-polyamide block copolymer described in International Publication No. WO2013012476, herein incorporated by reference in its entirety), and (poly(ethylene glycol))-(poly(propylene oxide))-(poly(ethylene glycol)) triblock copolymer (see e.g., U.S. Publication 20120121718 and U.S. Publication 20100003337 and U.S. Pat. No. 8,263,665, the contents of each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; the contents of which are herein incorporated by reference in their entirety). A non-limiting scalable method to produce nanoparticles which can penetrate human mucus is described by Xu et al. (see, e.g., J Control Release 2013, 170(2):279-86; the contents of which are herein incorporated by reference in their entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin 34 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see e.g., U.S. Publication 20100215580 and U.S. Publication 20080166414 and US20130164343; the contents of each of which are herein incorporated by reference in their entirety).

In some embodiments, the mucus penetrating lipid nanoparticles may comprise at least one polynucleotide described herein. The polynucleotide may be encapsulated in the lipid nanoparticle and/or disposed on the surface of the particle. The polynucleotide may be covalently coupled to the lipid nanoparticle. Formulations of mucus penetrating lipid nanoparticles may comprise a plurality of nanoparticles. Further, the formulations may contain particles which may interact with the mucus and alter the structural and/or adhesive properties of the surrounding mucus to decrease mucoadhesion, which may increase the delivery of the mucus penetrating lipid nanoparticles to the mucosal tissue.

In some embodiments, the mucus penetrating lipid nanoparticles may be a hypotonic formulation comprising a mucosal penetration enhancing coating. The formulation may be hypotonice for the epithelium to which it is being delivered. Non-limiting examples of hypotonic formulations may be found in International Patent Publication No. WO2013110028, the contents of which are herein incorporated by reference in their entirety.

In some embodiments, in order to enhance the delivery through the mucosal barrier the RNA (e.g., mRNA) vaccine formulation may comprise or be a hypotonic solution.

Hypotonic solutions were found to increase the rate at which mucoinert particles such as, but not limited to, mucus-penetrating particles, were able to reach the vaginal epithelial surface (see e.g., Ensign et al. Biomaterials 2013 34(28):6922-9, the contents of which are herein incorporated by reference in their entirety).

In some embodiments, the RNA (e.g., mRNA) vaccine is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT@(Cambridge, Mass.), and polyethylenimine (PEI) or protamine-based targeted and non-targeted delivery of nucleic acids acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132, the contents of each of which are incorporated herein by reference in their entirety).

In some embodiments, such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133, the contents of each of which are incorporated herein by reference in their entirety). One example of passive targeting of formulations to liver cells includes the DLin-DMA, DLin-KC2-DMA and DLin-MC3-DMA-based lipid nanoparticle formulations, which have been shown to bind to apolipoprotein E and promote binding and uptake of these formulations into hepatocytes in vivo (Akinc et al. Mol Ther. 2010 18:1357-1364, the contents of which are incorporated herein by reference in their entirety). Formulations can also be selectively targeted through expression of different ligands on their surface as exemplified by, but not limited by, folate, transferrin, N-acetylgalactosamine (GaINAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133, the contents of each of which are incorporated herein by reference in their entirety).

In some embodiments, the RNA (e.g., mRNA) vaccine is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In some embodiments, the lipid nanoparticle may be a self-assembly lipid-polymer nanoparticle (see Zhang et al., ACS Nano, 2008, 2 (8), pp 1696-1702; the contents of which are herein incorporated by reference in their entirety). As a non-limiting example, the SLN may be the SLN described in International Patent Publication No. WO2013105101, the contents of which are herein incorporated by reference in their entirety. As another non-limiting example, the SLN may be made by the methods or processes described in International Patent Publication No. WO2013105101, the contents of which are herein incorporated by reference in their entirety.

Liposomes, lipoplexes, or lipid nanoparticles may be used to improve the efficacy of polynucleotides directed protein production as these formulations may be able to increase cell transfection by the RNA (e.g., mRNA) vaccine; and/or increase the translation of encoded protein. One such example involves the use of lipid encapsulation to enable the effective systemic delivery of polyplex plasmid DNA (Heyes et al., Mol Ther. 2007 15:713-720; the contents of which are incorporated herein by reference in their entirety). The liposomes, lipoplexes, or lipid nanoparticles may also be used to increase the stability of the polynucleotide.

In some embodiments, the RNA (e.g., mRNA) vaccines of the present disclosure can be formulated for controlled release and/or targeted delivery. As used herein, “controlled release” refers to a pharmaceutical composition or compound release profile that conforms to a particular pattern of release to effect a therapeutic outcome. In some embodiments, the RNA (e.g., mRNA) vaccines may be encapsulated into a delivery agent described herein and/or known in the art for controlled release and/or targeted delivery. As used herein, the term “encapsulate” means to enclose, surround or encase. As it relates to the formulation of the compounds of the disclosure, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the disclosure may be enclosed, surrounded or encased within the delivery agent. “Partially encapsulation” means that less than 10, 10, 20, 30, 40 50 or less of the pharmaceutical composition or compound of the disclosure may be enclosed, surrounded or encased within the delivery agent. Advantageously, encapsulation may be determined by measuring the escape or the activity of the pharmaceutical composition or compound of the disclosure using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the disclosure are encapsulated in the delivery agent.

In some embodiments, the controlled release formulation may include, but is not limited to, tri-block co-polymers. As a non-limiting example, the formulation may include two different types of tri-block co-polymers (International Pub. No.

WO2012131104 and WO2012131106, the contents of each of which are incorporated herein by reference in their entirety).

In some embodiments, the RNA (e.g., mRNA) vaccines may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE@(Nanotherapeutics, Inc. Alachua, Fla.), HYLENEX@(Halozyme Therapeutics, San Diego Calif.), surgical sealants such as fibrinogen polymers (Ethicon Inc. Cornelia, Ga.), TISSELL@(Baxter International, Inc Deerfield, Ill.), PEG-based sealants, and COSEAL@(Baxter International, Inc Deerfield, Ill.).

In some embodiments, the lipid nanoparticle may be encapsulated into any polymer known in the art which may form a gel when injected into a subject. As another non-limiting example, the lipid nanoparticle may be encapsulated into a polymer matrix which may be biodegradable.

In some embodiments, the RNA (e.g., mRNA) vaccine formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In some embodiments, the RNA (e.g., mRNA) vaccine controlled release and/or targeted delivery formulation may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In some embodiments, the degradable polyesters may include a PEG conjugation to form a PEGylated polymer.

In some embodiments, the RNA (e.g., mRNA) vaccine controlled release and/or targeted delivery formulation comprising at least one polynucleotide may comprise at least one PEG and/or PEG related polymer derivatives as described in U.S. Pat. No. 8,404,222, the contents of which are incorporated herein by reference in their entirety.

In some embodiments, the RNA (e.g., mRNA) vaccine controlled release delivery formulation comprising at least one polynucleotide may be the controlled release polymer system described in US20130130348, the contents of which are incorporated herein by reference in their entirety.

In some embodiments, the RNA (e.g., mRNA) vaccines of the present disclosure may be encapsulated in a therapeutic nanoparticle, referred to herein as “therapeutic nanoparticle RNA (e.g., mRNA) vaccines.” Therapeutic nanoparticles may be formulated by methods described herein and known in the art such as, but not limited to, International Pub Nos. WO2010005740, WO2010030763, WO2010005721, WO2010005723, WO2012054923, U.S. Publication Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211; the contents of each of which are herein incorporated by reference in their entirety. In some embodiments, therapeutic polymer nanoparticles may be identified by the methods described in US Pub No. US20120140790, the contents of which are herein incorporated by reference in their entirety.

In some embodiments, the therapeutic nanoparticle RNA (e.g., mRNA) vaccine may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the polynucleotides of the present disclosure (see International Pub No. 2010075072 and US Pub No. US20100216804, US20110217377 and US20120201859, the contents of each of which are incorporated herein by reference in their entirety). In another non-limiting example, the sustained release formulation may comprise agents which permit persistent bioavailability such as, but not limited to, crystals, macromolecular gels and/or particulate suspensions (see U.S. Patent Publication No US20130150295, the contents of each of which are incorporated herein by reference in their entirety).

In some embodiments, the therapeutic nanoparticle RNA (e.g., mRNA) vaccines may be formulated to be target specific. As a non-limiting example, the therapeutic nanoparticles may include a corticosteroid (see International Pub. No. WO2011084518, the contents of which are incorporated herein by reference in their entirety). As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, the contents of each of which are incorporated herein by reference in their entirety.

In some embodiments, the nanoparticles of the present disclosure may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In some embodiments, the therapeutic nanoparticle comprises a diblock copolymer. In some embodiments, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In yet another embodiment, the diblock copolymer may be a high-X diblock copolymer such as those described in International Patent Publication No. WO2013120052, the contents of which are incorporated herein by reference in their entirety.

As a non-limiting example the therapeutic nanoparticle comprises a PLGA-PEG block copolymer (see U.S. Publication No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the contents of each of which are herein incorporated by reference in their entirety). In yet another non-limiting example, the therapeutic nanoparticle is a stealth nanoparticle or a target-specific stealth nanoparticle as described in U.S. Patent Publication No. US20130172406, the contents of which are herein incorporated by reference in their entirety.

In some embodiments, the therapeutic nanoparticle may comprise a multiblock copolymer (see e.g., U.S. Pat. Nos. 8,263,665 and 8,287,910 and U.S. Patent Pub. No. US20130195987, the contents of each of which are herein incorporated by reference in their entirety).

In yet another non-limiting example, the lipid nanoparticle comprises the block copolymer PEG-PLGA-PEG (see e.g., the thermosensitive hydrogel (PEG-PLGA-PEG) was used as a TGF-beta1 gene delivery vehicle in Lee et al. Thermosensitive Hydrogel as a Tgf-β1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253, the contents of each of which are herein incorporated by reference in their entirety). The RNA (e.g., mRNA) vaccines of the present disclosure may be formulated in lipid nanoparticles comprising the PEG-PLGA-PEG block copolymer.

In some embodiments, the therapeutic nanoparticle may comprise a multiblock copolymer (see e.g., U.S. Pat. Nos. 8,263,665 and 8,287,910 and U.S. Patent Pub. No. US20130195987, the contents of each of which are herein incorporated by reference in their entirety).

In some embodiments, the block copolymers described herein may be included in a polyion complex comprising a non-polymeric micelle and the block copolymer. (see e.g., U.S. Publication No. 20120076836, the contents of which are herein incorporated by reference in their entirety).

In some embodiments, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In some embodiments, the therapeutic nanoparticles may comprise at least one poly(vinyl ester) polymer. The poly(vinyl ester) polymer may be a copolymer such as a random copolymer. As a non-limiting example, the random copolymer may have a structure such as those described in International Application No. WO2013032829 or U.S. Patent Publication No US20130121954, the contents of each of which are herein incorporated by reference in their entirety. In some embodiments, the poly(vinyl ester) polymers may be conjugated to the polynucleotides described herein.

In some embodiments, the therapeutic nanoparticle may comprise at least one diblock copolymer. The diblock copolymer may be, but it not limited to, a poly(lactic) acid-poly(ethylene)glycol copolymer (see, e.g., International Patent Publication No. WO2013044219, the contents of which are herein incorporated by reference in their entirety).

As a non-limiting example, the therapeutic nanoparticle may be used to treat cancer (see International publication No. WO2013044219, the contents of which are herein incorporated by reference in their entirety).

In some embodiments, the therapeutic nanoparticles may comprise at least one cationic polymer described herein and/or known in the art.

In some embodiments, the therapeutic nanoparticles may comprise at least one amine-containing polymer such as, but not limited to polylysine, polyethylene imine, poly(amidoamine) dendrimers, poly(beta-amino esters) (see, e.g., U.S. Pat. No. 8,287,849, the contents of which are herein incorporated by reference in their entirety) and combinations thereof.

In some embodiments, the nanoparticles described herein may comprise an amine cationic lipid such as those described in International Patent Application No. WO2013059496, the contents of which are herein incorporated by reference in their entirety. In some embodiments, the cationic lipids may have an amino-amine or an amino-amide moiety.

In some embodiments, the therapeutic nanoparticles may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In some embodiments, the degradable polyesters may include a PEG conjugation to form a PEGylated polymer.

In some embodiments, the synthetic nanocarriers may contain an immunostimulatory agent to enhance the immune response from delivery of the synthetic nanocarrier. As a non-limiting example, the synthetic nanocarrier may comprise a Th1 immunostimulatory agent, which may enhance a Th1-based response of the immune system (see International Pub No. WO2010123569 and U.S. Publication No. US20110223201, the contents of each of which are herein incorporated by reference in their entirety).

In some embodiments, the synthetic nanocarriers may be formulated for targeted release. In some embodiments, the synthetic nanocarrier is formulated to release the polynucleotides at a specified pH and/or after a desired time interval. As a non-limiting example, the synthetic nanoparticle may be formulated to release the RNA (e.g., mRNA) vaccines after 24 hours and/or at a pH of 4.5 (see International Publication Nos. WO2010138193 and WO2010138194 and US Pub Nos. US20110020388 and US20110027217, each of which is herein incorporated by reference in their entireties).

In some embodiments, the synthetic nanocarriers may be formulated for controlled and/or sustained release of the polynucleotides described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In some embodiments, the RNA (e.g., mRNA) vaccine may be formulated for controlled and/or sustained release wherein the formulation comprises at least one polymer that is a crystalline side chain (CYSC) polymer. CYSC polymers are described in U.S. Pat. No. 8,399,007, herein incorporated by reference in its entirety.

In some embodiments, the synthetic nanocarrier may be formulated for use as a vaccine. In some embodiments, the synthetic nanocarrier may encapsulate at least one polynucleotide which encode at least one antigen. As a non-limiting example, the synthetic nanocarrier may include at least one antigen and an excipient for a vaccine dosage form (see International Publication No. WO2011150264 and U.S. Publication No. US20110293723, the contents of each of which are herein incorporated by reference in their entirety). As another non-limiting example, a vaccine dosage form may include at least two synthetic nanocarriers with the same or different antigens and an excipient (see International Publication No. WO2011150249 and U.S. Publication No. US20110293701, the contents of each of which are herein incorporated by reference in their entirety). The vaccine dosage form may be selected by methods described herein, known in the art and/or described in International Publication No. WO2011150258 and U.S. Publication No. US20120027806, the contents of each of which are herein incorporated by reference in their entirety).

In some embodiments, the synthetic nanocarrier may comprise at least one polynucleotide which encodes at least one adjuvant. As non-limiting example, the adjuvant may comprise dimethyldioctadecylammonium-bromide, dimethyldioctadecylammonium-chloride, dimethyldioctadecylammonium-phosphate or dimethyldioctadecylammonium-acetate (DDA) and an apolar fraction or part of said apolar fraction of a total lipid extract of a mycobacterium (see, e.g., U.S. Pat. No. 8,241,610, the content of which is herein incorporated by reference in its entirety). In some embodiments, the synthetic nanocarrier may comprise at least one polynucleotide and an adjuvant. As a non-limiting example, the synthetic nanocarrier comprising and adjuvant may be formulated by the methods described in International Publication No. WO2011150240 and U.S. Publication No. US20110293700, the contents of each of which are herein incorporated by reference in their entirety.

In some embodiments, the synthetic nanocarrier may encapsulate at least one polynucleotide that encodes a peptide, fragment or region from a virus. As a non-limiting example, the synthetic nanocarrier may include, but is not limited to, any of the nanocarriers described in International Publication No. WO2012024621, WO201202629, WO2012024632 and U.S. Publication No. US20120064110, US20120058153 and US20120058154, the contents of each of which are herein incorporated by reference in their entirety.

In some embodiments, the synthetic nanocarrier may be coupled to a polynucleotide which may be able to trigger a humoral and/or cytotoxic T lymphocyte (CTL) response (see, e.g., International Publication No. WO2013019669, the contents of which are herein incorporated by reference in their entirety).

In some embodiments, the RNA (e.g., mRNA) vaccine may be encapsulated in, linked to and/or associated with zwitterionic lipids. Non-limiting examples of zwitterionic lipids and methods of using zwitterionic lipids are described in U.S. Patent Publication No. US20130216607, the contents of which are herein incorporated by reference in their entirety.

In some embodiment, the zwitterionic lipids may be used in the liposomes and lipid nanoparticles described herein.

In some embodiments, the RNA (e.g., mRNA) vaccine may be formulated in colloid nanocarriers as described in U.S. Patent Publication No. US20130197100, the contents of which are herein incorporated by reference in their entirety.

In some embodiments, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Publication No. 20120282343, the contents of which are herein incorporated by reference in their entirety.

In some embodiments, LNPs comprise the lipid KL52 (an amino-lipid disclosed in U.S. Application Publication No. 2012/0295832, the contents of which are herein incorporated by reference in their entirety. Activity and/or safety (as measured by examining one or more of ALT/AST, white blood cell count and cytokine induction, for example) of LNP administration may be improved by incorporation of such lipids. LNPs comprising KL52 may be administered intravenously and/or in one or more doses. In some embodiments, administration of LNPs comprising KL52 results in equal or improved mRNA and/or protein expression as compared to LNPs comprising MC3.

In some embodiments, RNA (e.g., mRNA) vaccine may be delivered using smaller LNPs. Such particles may comprise a diameter from below 0.1 um up to 100 nm such as, but not limited to, less than 0.1 um, less than 1.0 um, less than 5 um, less than 10 um, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, less than 975 um, or less than 1000 um.

In some embodiments, RNA (e.g., mRNA) vaccines may be delivered using smaller LNPs, which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nm, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

In some embodiments, such LNPs are synthesized using methods comprising microfluidic mixers. Examples of microfluidic mixers may include, but are not limited to, a slit interdigital micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. 1:e37; Chen, D. et al., Rapid discovery of potent siRNA-containing lipid nanoparticles enabled by controlled microfluidic formulation. J Am Chem Soc. 2012. 134(16):6948-51, the contents of each of which are herein incorporated by reference in their entirety). In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Application Publication Nos. 2004/0262223 and 2012/0276209, the contents of each of which are herein incorporated by reference in their entirety.

In some embodiments, the RNA (e.g., mRNA) vaccine of the present disclosure may be formulated in lipid nanoparticles created using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (IJMM) from the Institut fiir Mikrotechnik Mainz GmbH, Mainz Germany).

In some embodiments, the RNA (e.g., mRNA) vaccines of the present disclosure may be formulated in lipid nanoparticles created using microfluidic technology (see, e.g., Whitesides, George M. The Origins and the Future of Microfluidics. Nature, 2006 442: 368-373; and Abraham et al. Chaotic Mixer for Microchannels. Science, 2002 295: 647-651; each of which is herein incorporated by reference in its entirety). As a non-limiting example, controlled microfluidic formulation includes a passive method for mixing streams of steady pressure-driven flows in micro channels at a low Reynolds number (see, e.g., Abraham et al. Chaotic Mixer for Microchannels. Science, 2002 295: 647-651, the contents of which are herein incorporated by reference in their entirety).

In some embodiments, the RNA (e.g., mRNA) vaccines of the present disclosure may be formulated in lipid nanoparticles created using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In some embodiments, the RNA (e.g., mRNA) vaccines of the disclosure may be formulated for delivery using the drug encapsulating microspheres described in International Patent Publication No. WO2013063468 or U.S. Pat. No. 8,440,614, the contents of each of which are herein incorporated by reference in their entirety. The microspheres may comprise a compound of the formula (I), (II), (III), (IV), (V) or (VI) as described in International Patent Publication No. WO2013063468, the contents of which are herein incorporated by reference in their entirety. In some embodiments, the amino acid, peptide, polypeptide, lipids (APPL) are useful in delivering the RNA (e.g., mRNA) vaccines of the disclosure to cells (see International Patent Publication No. WO2013063468, the contents of which are herein incorporated by reference in their entirety).

In some embodiments, the RNA (e.g., mRNA) vaccines of the disclosure may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles may have a diameter from about 10 to 500 nm.

In some embodiments, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the lipid nanoparticle may be a limit size lipid nanoparticle described in International Patent Publication No. WO2013059922, the contents of which are herein incorporated by reference in their entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In some embodiments, the limit size lipid nanoparticle may comprise a polyethylene glycol-lipid such as, but not limited to, DLPE-PEG, DMPE-PEG, DPPC-PEG and DSPE-PEG.

In some embodiments, the RNA (e.g., mRNA) vaccines may be delivered, localized and/or concentrated in a specific location using the delivery methods described in International Patent Publication No. WO2013063530, the contents of which are herein incorporated by reference in their entirety. As a non-limiting example, a subject may be administered an empty polymeric particle prior to, simultaneously with or after delivering the RNA (e.g., mRNA) vaccines to the subject. The empty polymeric particle undergoes a change in volume once in contact with the subject and becomes lodged, embedded, immobilized or entrapped at a specific location in the subject.

In some embodiments, the RNA (e.g., mRNA) vaccines may be formulated in an active substance release system (see, e.g., U.S. Patent Publication No. US20130102545, the contents of which are herein incorporated by reference in their entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., polynucleotides described herein), where the therapeutically active substance is released by the cleavage of the substrate molecule by the catalytically active nucleic acid.

In some embodiments, the RNA (e.g., mRNA) vaccines may be formulated in a nanoparticle comprising an inner core comprising a non-cellular material and an outer surface comprising a cellular membrane. The cellular membrane may be derived from a cell or a membrane derived from a virus. As a non-limiting example, the nanoparticle may be made by the methods described in International Patent Publication No. WO2013052167, the contents of which are herein incorporated by reference in their entirety. As another non-limiting example, the nanoparticle described in International Patent Publication No. WO2013052167, the contents of which are herein incorporated by reference in their entirety, may be used to deliver the RNA (e.g., mRNA) vaccines described herein.

In some embodiments, the RNA (e.g., mRNA) vaccines may be formulated in porous nanoparticle-supported lipid bilayers (protocells). Protocells are described in International Patent Publication No. WO2013056132, the contents of which are herein incorporated by reference in their entirety.

In some embodiments, the RNA (e.g., mRNA) vaccines described herein may be formulated in polymeric nanoparticles as described in or made by the methods described in U.S. Pat. Nos. 8,420,123 and 8,518,963 and European Patent No. EP2073848B1, the contents of each of which are herein incorporated by reference in their entirety. As a non-limiting example, the polymeric nanoparticle may have a high glass transition temperature such as the nanoparticles described in or nanoparticles made by the methods described in U.S. Pat. No. 8,518,963, the contents of which are herein incorporated by reference in their entirety. As another non-limiting example, the polymer nanoparticle for oral and parenteral formulations may be made by the methods described in European Patent No. EP2073848B1, the contents of which are herein incorporated by reference in their entirety.

In some embodiments, the RNA (e.g., mRNA) vaccines described herein may be formulated in nanoparticles used in imaging. The nanoparticles may be liposome nanoparticles such as those described in U.S. Patent Publication No US20130129636, herein incorporated by reference in its entirety. As a non-limiting example, the liposome may comprise gadolinium(III)2-{4,7-bis-carboxymethyl-10-[(N,N-distearylamidomethyl-N′-amido-methyl]-1,4,7,10-tetra-azacyclododec-1-yl}-acetic acid and a neutral, fully saturated phospholipid component (see, e.g., U.S. Patent Publication No US20130129636, the contents of which are herein incorporated by reference in their entirety).

In some embodiments, the nanoparticles which may be used in the present disclosure are formed by the methods described in U.S. Patent Application No. US20130130348, the contents of which are herein incorporated by reference in their entirety.

The nanoparticles of the present disclosure may further include nutrients such as, but not limited to, those which deficiencies can lead to health hazards from anemia to neural tube defects (see, e.g., the nanoparticles described in International Patent Publication No WO2013072929, the contents of which are herein incorporated by reference in their entirety). As a non-limiting example, the nutrient may be iron in the form of ferrous, ferric salts or elemental iron, iodine, folic acid, vitamins or micronutrients.

In some embodiments, the RNA (e.g., mRNA) vaccines of the present disclosure may be formulated in a swellable nanoparticle. The swellable nanoparticle may be, but is not limited to, those described in U.S. Pat. No. 8,440,231, the contents of which are herein incorporated by reference in their entirety. As a non-limiting embodiment, the swellable nanoparticle may be used for delivery of the RNA (e.g., mRNA) vaccines of the present disclosure to the pulmonary system (see, e.g., U.S. Pat. No. 8,440,231, the contents of which are herein incorporated by reference in their entirety).

The RNA (e.g., mRNA) vaccines of the present disclosure may be formulated in polyanhydride nanoparticles such as, but not limited to, those described in U.S. Pat. No. 8,449,916, the contents of which are herein incorporated by reference in their entirety.

The nanoparticles and microparticles of the present disclosure may be geometrically engineered to modulate macrophage and/or the immune response. In some embodiments, the geometrically engineered particles may have varied shapes, sizes and/or surface charges in order to incorporated the polynucleotides of the present disclosure for targeted delivery such as, but not limited to, pulmonary delivery (see, e.g., International Publication No WO2013082111, the contents of which are herein incorporated by reference in their entirety). Other physical features the geometrically engineering particles may have include, but are not limited to, fenestrations, angled arms, asymmetry and surface roughness, charge which can alter the interactions with cells and tissues. As a non-limiting example, nanoparticles of the present disclosure may be made by the methods described in International Publication No WO2013082111, the contents of which are herein incorporated by reference in their entirety.

In some embodiments, the nanoparticles of the present disclosure may be water soluble nanoparticles such as, but not limited to, those described in International Publication No. WO2013090601, the contents of which are herein incorporated by reference in their entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In some embodiments the nanoparticles of the present disclosure may be developed by the methods described in U.S. Patent Publication No. US20130172406, the contents of which are herein incorporated by reference in their entirety.

In some embodiments, the nanoparticles of the present disclosure are stealth nanoparticles or target-specific stealth nanoparticles such as, but not limited to, those described in U.S. Patent Publication No. US20130172406, the contents of which are herein incorporated by reference in their entirety. The nanoparticles of the present disclosure may be made by the methods described in U.S. Patent Publication No. US20130172406, the contents of which are herein incorporated by reference in their entirety.

In some embodiments, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In some embodiments, the nanoparticle may be a nanoparticle-nucleic acid hybrid structure having a high density nucleic acid layer. As a non-limiting example, the nanoparticle-nucleic acid hybrid structure may made by the methods described in U.S. Patent Publication No. US20130171646, the contents of which are herein incorporated by reference in their entirety. The nanoparticle may comprise a nucleic acid such as, but not limited to, polynucleotides described herein and/or known in the art.

At least one of the nanoparticles of the present disclosure may be embedded in in the core a nanostructure or coated with a low density porous 3-D structure or coating which is capable of carrying or associating with at least one payload within or on the surface of the nanostructure. Non-limiting examples of the nanostructures comprising at least one nanoparticle are described in International Patent Publication No. WO2013123523, the contents of which are herein incorporated by reference in their entirety.

In other embodiments the nucleic acid vaccine is chemically modified and in other embodiments the nucleic acid vaccine is not chemically modified.

In some embodiments, the antibody titer produced by the mRNA vaccines of the invention is a neutralizing antibody titer. In some embodiments the neutralizing antibody titer is greater than a protein vaccine. In other embodiments the neutralizing antibody titer produced by the mRNA vaccines of the invention is greater than an adjuvanted protein vaccine. In yet other embodiments the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

Also provided are nucleic acid vaccines comprising one or more RNA polynucleotides having an open reading frame encoding a first antigenic polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the polypeptide. In some embodiments, the RNA polynucleotide is formulated to produce a neutralizing antibodies within one week of a single administration. In some embodiments, the adjuvant is selected from a cationic peptide and an immunostimulatory nucleic acid. In some embodiments, the cationic peptide is protamine.

The present invention provides nucleic acid vaccines comprising one or more RNA polynucleotides having an open reading frame comprising at least one chemical modification or optionally no nucleotide modification, the open reading frame encoding a first antigenic polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the antigenic polypeptide.

Other embodiments provide nucleic acid vaccines comprising one or more RNA polynucleotides having an open reading frame comprising at least one chemical modification or optionally no nucleotide modification, the open reading frame encoding a antigenic polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Embodiments of the invention also provide a unit of use vaccine, comprising between 10 ug and 400 ug of one or more RNA polynucleotides having an open reading frame comprising at least one chemical modification or optionally no nucleotide modification, the open reading frame encoding a antigenic polypeptide, and a pharmaceutically acceptable carrier or excipient, formulated for delivery to a human subject. In some embodiments, the vaccine further comprises a cationic lipid nanoparticle.

Other embodiments provide nucleic acid vaccines comprising one or more RNA polynucleotides as described herein, having at least one chemical modification, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Other embodiments provide nucleic acid vaccines comprising an LNP (lipid nanoparticle) formulated RNA polynucleotide having sequence comprising no nucleotide modifications (unmodified), the polynucleotide encoding an RBD as described herein, wherein the vaccine has at least 10 fold less RNA polynucleotide than is required for an unmodified mRNA vaccine not formulated in a LNP to produce an equivalent antibody titer. In some embodiments, the RNA polynucleotide is present in a dosage of 25-100 micrograms.

In some embodiments, the disclosure features a pharmaceutical composition comprising a nanoparticle composition according to the preceding embodiments and a pharmaceutically acceptable carrier. The pharmaceutically acceptable carrier may be as described herein, and may also include one or more agents for facilitating storage of the composition at low temperatures. For example, the pharmaceutical composition may be refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition is a solution that is refrigerated for storage and/or shipment at, for example, about −20° C., −30° C., −40° C., −50° C., −60° C., −70° C., or −80° C. Accordingly, it will be appreciated that the compositions described herein may further comprise one or more cryoprotectants or cryopreservatives. Optionally the cryopreservative or cryoprotectant may comprise a sugar such as sucrose, glucose or related sugar-based cryoprotectant.

### Subjects and Methods of Administration

The present invention also provides uses of the polynucleotides and compositions of the invention for producing an antigen-specific immune response in a subject. Such methods typically comprise administering a polynucleotide of the invention, preferably formulated in a lipid nanoparticle as described herein, to a subject in need thereof.

A subject or individual in need of treatment according to any embodiment of the invention, or requiring administration of any composition described herein, may be an individual who is susceptible to infection by coronavirus and/or susceptible to diseases or disorders caused by coronavirus infection. A subject of this invention can be a mammal and in particular embodiments is a human, which can be an infant, a child, an adult or an elderly adult.

A “subject at risk of infection by a coronavirus” or a “subject at risk of coronavirus infection” is any subject who may be or has been exposed to a coronavirus. “Subject” or “individual” includes any human or non-human animal. Thus, in addition to being useful for human treatment, the polynucleotide, vector, nanoparticle or composition of the present invention may also be useful for veterinary treatment of mammals, including companion animals and farm animals, such as, but not limited to dogs, cats, horses, cows, sheep, and pigs, or any animal that can be infected by coronavirus.

The subjects at risk include, but are not limited to, an immunocompromised person, an elderly adult (more than 65 years of age), children younger than 2 years of age, healthcare workers, adults or children in close contact with a person(s) with confirmed or suspected coronavirus infection, and people with underlying medical conditions such as pulmonary infection, heart disease or diabetes, primary or secondary immunodeficiency.

In some embodiments, following administration of a polynucleotide or composition of the invention, the subject exhibits a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, a polynucleotide (e.g., mRNA) vaccine is administered to a subject by intradermal or intramuscular injection.

In some embodiments of the present disclosure, there is provided methods of inducing an antigen specific immune response in a subject, including administering to a subject a RNA (e.g., mRNA) vaccine as described herein, in an effective amount to produce an antigen specific immune response in a subject. Antigen-specific immune responses in a subject may be determined, in some embodiments, by assaying for antibody titer following administration to the subject of any of the polynucleotide (e.g., mRNA) vaccines of the present disclosure. In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2-10 times relative to a control.

In some embodiments, the control is an anti-antigenic polypeptide antibody titer produced in a subject who has not been administered a RNA (e.g., mRNA) vaccine of the present disclosure. In some embodiments, the control is an anti-antigenic polypeptide antibody titer produced in a subject who has been administered a live attenuated or inactivated coronavirus vaccine (see, e.g., Ren J. et al. J of Gen. Virol. 2015; 96: 1515-1520), or wherein the control is an anti-antigenic polypeptide antibody titer produced in a subject who has been administered a recombinant or purified coronavirus protein vaccine. In some embodiments, the control is an anti-antigenic polypeptide antibody titer produced in a subject who has been administered coronavirus virus-like particle (VLP) vaccine (see, e.g., Cox R G et al., J Virol. 2014 June; 88(11): 6368-6379).

A polynucleotide (e.g., mRNA) vaccine of the present disclosure is administered to a subject in an effective amount (an amount effective to induce an immune response). In some embodiments, the effective amount is a dose equivalent to an at least 2-fold, at least 4-fold, at least 10-fold, at least 100-fold, at least 1000-fold reduction in the standard of care dose of a recombinant coronavirus protein vaccine, wherein the anti-antigenic polypeptide antibody titer produced in the subject is equivalent to an anti-antigenic polypeptide antibody titer produced in a control subject administered the standard of care dose of a recombinant coronavirus protein vaccine, a purified coronavirus protein vaccine, a live attenuated coronavirus vaccine, an inactivated coronavirus vaccine, or a coronavirus VLP vaccine. In some embodiments, the effective amount is a dose equivalent to 2-1000-fold reduction in the standard of care dose of a recombinant coronavirus protein vaccine, wherein the anti-antigenic polypeptide antibody titer produced in the subject is equivalent to an anti-antigenic polypeptide antibody titer produced in a control subject administered the standard of care dose of a recombinant coronavirus protein vaccine, a purified coronavirus protein vaccine, a live attenuated coronavirus vaccine, an inactivated h coronavirus vaccine, or a coronavirus VLP vaccine.

In some embodiments, the control is an anti-antigenic polypeptide antibody titer produced in a subject who has been administered a virus-like particle (VLP) vaccine comprising structural proteins of a coronavirus.

In some embodiments, the polynucleotide (e.g., mRNA) vaccine is formulated in an effective amount to produce an antigen specific immune response in a subject.

In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times.

In some embodiments, the efficacy (or effectiveness) of a polynucleotide (e.g., mRNA) vaccine is greater than 60%. In some embodiments, the efficacy (or effectiveness) of a polynucleotide (e.g., mRNA) vaccine is at least 65%, at least 70%, at least 75%, at least 80%, at least 85%, or at least 90%.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

$$Efficacy = ( A R U - A RV ) / ARU \times 100 ; and$$

$$Efficacy = ( 1 - R R ) \times 100 .$$

Similarly, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

$$Effectiveness = ( 1 - O R ) \times 100 .$$

In some embodiments, the vaccine immunizes the subject against a coronavirus for up to 2 years. In some embodiments, the vaccine immunizes the subject against a coronavirus for more than 2 years, more than 3 years, more than 4 years, or for 5-10 years.

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a RNA (e.g., mRNA) vaccine may be administered later in life, for example, at the age of about 6 months to about 5 years, or older.

In some embodiments, the subject is pregnant (e.g., in the first, second or third trimester) when administered an RNA (e.g., mRNA) vaccine.

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject is has a chronic pulmonary disease (e.g., chronic obstructive pulmonary disease (COPD) or asthma). Two forms of COPD include chronic bronchitis, which involves a long-term cough with mucus, and emphysema, which involves damage to the lungs over time. Thus, a subject administered a polynucleotide (e.g., mRNA) vaccine may have chronic bronchitis or emphysema.

In some embodiments, the subject is immunocompromised (has an impaired immune system, e.g., has an immune disorder or autoimmune disorder).

Yet other embodiments provide compositions for and methods of vaccinating a subject comprising administering to the subject a nucleic acid vaccine comprising one or more RNA polynucleotides, wherein the RNA polynucleotide does not include a stabilization element, and wherein an adjuvant is not coformulated or co-administered with the vaccine.

In other embodiments the invention is a composition for or method of vaccinating a subject comprising administering to the subject a nucleic acid vaccine comprising one or more RNA polynucleotides having an open reading frame encoding a first antigenic polypeptide wherein a dosage of between 10 μg/kg and 400 μg/kg of the nucleic acid vaccine is administered to the subject. In some embodiments the dosage of the RNA polynucleotide is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid vaccine is administered to the subject by intradermal or intramuscular injection. In some embodiments, the nucleic acid vaccine is administered to the subject on day zero. In some embodiments, a second dose of the nucleic acid vaccine is administered to the subject on day twenty one.

In some embodiments, a dosage of 25 micrograms (μg) of the RNA polynucleotide is included in the nucleic acid vaccine administered to the subject. In some embodiments, a dosage of 100 micrograms (μg) of the RNA polynucleotide is included in the nucleic acid vaccine administered to the subject. In some embodiments, a dosage of 50 micrograms (μg) of the RNA polynucleotide is included in the nucleic acid vaccine administered to the subject. In some embodiments, a dosage of 75 micrograms (μg) of the RNA polynucleotide is included in the nucleic acid vaccine administered to the subject. In some embodiments, a dosage of 150 micrograms (μg) of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms (μg) of the RNA polynucleotide is included in the nucleic acid vaccine administered to the subject. In some embodiments, the RNA polynucleotide accumulates at a 100 fold higher level in the local lymph node in comparison with the distal lymph node.

Embodiments of the invention provide methods of creating, maintaining or restoring antigenic memory to a respiratory virus strain in an individual or population of individuals comprising administering to said individual or population an antigenic memory booster nucleic acid vaccine comprising (a) at least one RNA polynucleotide, said polynucleotide comprising at least one chemical modification or optionally no nucleotide modification and two or more codon-optimized open reading frames, said open reading frames encoding a set of reference antigenic polypeptides, and (b) optionally a pharmaceutically acceptable carrier or excipient. In some embodiments, the vaccine is administered to the individual via a route selected from the group consisting of intramuscular administration, intradermal administration and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

Embodiments of the invention provide methods of vaccinating a subject comprising administering to the subject a single dosage of between 25 μg/kg and 400 μg/kg of a nucleic acid vaccine comprising one or more RNA polynucleotides as described herein, in an effective amount to vaccinate the subject.

In other embodiments the invention encompasses a method of treating an elderly subject age 60 years or older comprising administering to the subject a nucleic acid vaccine comprising one or more RNA polynucleotides having an open reading frame encoding a respiratory virus antigenic polypeptide in an effective amount to vaccinate the subject.

In other embodiments the invention encompasses a method of treating a young subject age 17 years or younger comprising administering to the subject a nucleic acid vaccine comprising one or more RNA polynucleotides having an open reading frame encoding a respiratory virus antigenic polypeptide in an effective amount to vaccinate the subject.

In other embodiments the invention encompasses a method of treating an adult subject comprising administering to the subject a nucleic acid vaccine comprising one or more RNA polynucleotides having an open reading frame encoding a respiratory virus antigenic polypeptide in an effective amount to vaccinate the subject.

In preferred embodiments, vaccines of the invention (e.g., LNP-encapsulated mRNA vaccines) produce prophylactically- and/or therapeutically-efficacious levels, concentrations and/or titers of antigen-specific antibodies in the blood or serum of a vaccinated subject.

As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result.

In exemplary embodiments, antibody titer is determined or measured by enzyme-linked immunosorbent assay (ELISA). In exemplary embodiments, antibody titer is determined or measured by neutralization assay, e.g., by microneutralization assay. In certain embodiments, antibody titer measurement is expressed as a ratio, such as 1:40, 1:100, etc. In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.) In exemplary embodiments of the invention, antigen-specific antibodies are measured in units of μg/ml or are measured in units of IU/L (International Units per liter) or mlU/ml (milli International Units per ml). In exemplary embodiments of the invention, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the invention, an efficacious vaccine produces >10 mlU/ml, >20 mlU/ml, >50 mlU/ml, >100 mlU/ml, >200 mlU/ml, >500 mlU/ml or >1000 mlU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA).

In exemplary embodiments, antibody level or concentration is determined or measured by neutralization assay, e.g., by microneutralization assay.

### EXAMPLES

### Example 1: Production of RBD-TM RNA and Protein

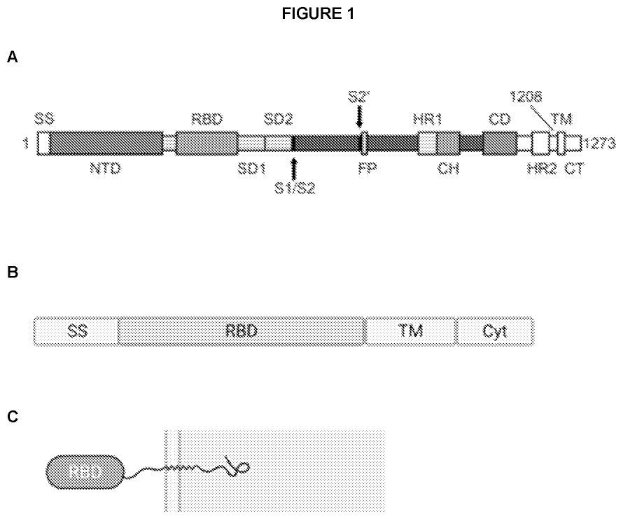

The domain architecture of the coronavirus spike protein is shown in FIG. 1A. The total length of SARS-CoV-2 S protein is 1273 aa and consists of a signal peptide (amino acids 1-13) located at the N-terminus, the S1 subunit (14-685 residues), and the S2 subunit (686-1273 residues). The S1 subunit, comprises an N-terminal domain (14-305 residues) and a receptor-binding domain (RBD, 319-541 residues). The S2 domain comprises a fusion peptide (FP) (788-806 residues), heptapeptide repeat sequence 1 (HR1) (912-984 residues), HR2 (1163-1213 residues), TM domain (1213-1237 residues), and cytoplasm domain (1237-1273 residues). FIGS. 1B and 1C show the general structure of a preferred mRNA vaccine of the invention, comprising the sequence of the receptor binding domain (RBD) of the S1 subunit of the spike protein, fused to a transmembrane domain (RBD-TM), optionally also comprising a cytoplasmic domain.

A standard template (designed in house) or Self-Amplifying template (VEEV) template was used to produce the open reading frame (ORF) of RBD-TM.

mRNA was produced using ARCA Hiscribe kit (NEB) as recommended by the manufacturer. In some instances, mRNA was generated from a PCR template containing UTRs, polyA100 and T7 region.

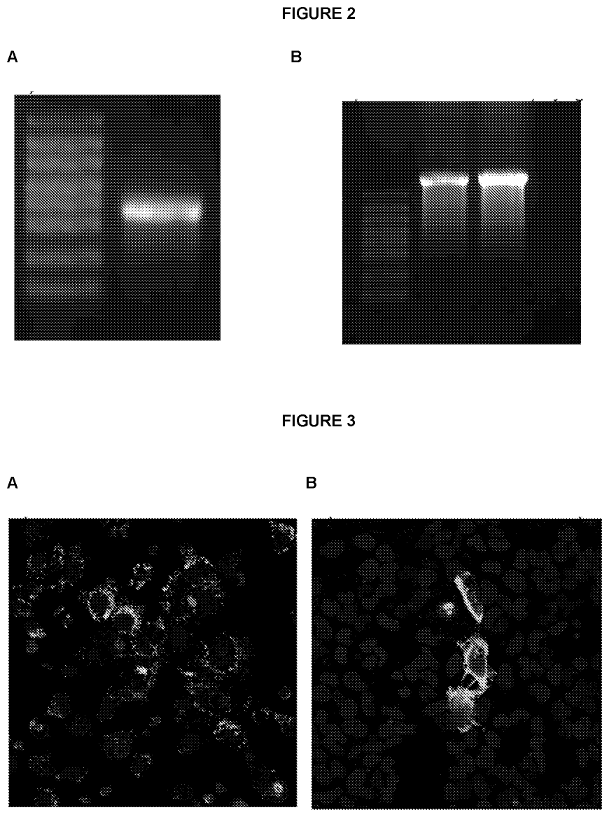

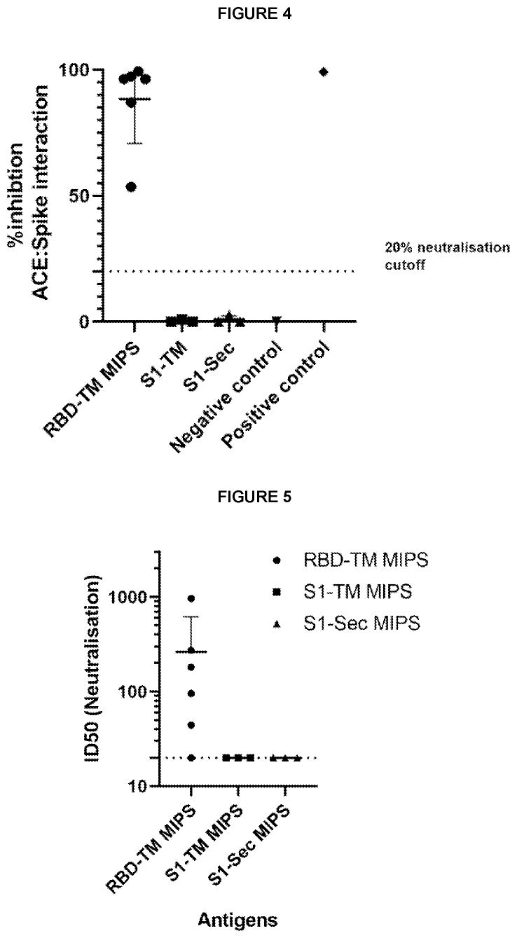

After mRNA production, mRNA was treated with DNase I and precipitated using LiC₁₂. mRNA was visualised using denaturing RNA gel (formaldehyde gel) mRNA stained with Ethidium bromide and visualised using BioRad Chemdoc imager. FIG. 2 A shows mRNA from a standard RBD-TM template designed in house. FIG. 2B shows mRNA generated from Self-amplifying mRNA (VEEV). 250-500 ng of mRNA was loaded into each well using gel-loading dye (Thermofisher) and a riboruler high-range (Thermofisher) was used as a ladder. RBD-TM mRNA was transfected with lipofectamine 3000 into 8-well microslide (Ibidi). 24 hours after transfection, cells were stained with primary anti-Spike antibodies (mouse) and secondary Alexaflour 488 antibody (anti-mouse). Cells were imaged using SP8 inverted microscope (FIG. 3). Green cells indicates cells expressing the RBD (labelled). Hoechst dye was used as a nuclear stain (blue).

### Generation of Lipid Nanoparticles

Nanoparticles can be made with mixing processes such as microfluidics and T-junction mixing of two fluid streams, one of which contains the therapeutic and/or prophylactic and the other has the lipid components. Lipid compositions are prepared by combining a cationic lipid (such as DODAC, DODAP or Dlin-MC3-DMA), a phospholipid (such as DOPE or DSPC) a PEG lipid (such as 1,2-dimyristoyl-rac-glycero-3-methoxypolyethylene glycol-2000, also known as DMG-PEG) and a structural lipid (such as cholesterol or a corticosteroid (such as prednisolone, dexamethasone, prednisone, and hydrocortisone), or a combination thereof) at concentrations of about 50 mM in ethanol. Unless specified otherwise, the nanoparticle referred to as “MIPS-LNP” herein includes any one of the lipid nanoparticles 1 to 4 referred to below or in further Examples herein Solutions should be refrigerated for storage at, for example, 4° C. or frozen for storage at, for example −20° C. Lipids are combined to yield desired molar ratios and diluted with ethanol to a final lipid concentration of between about 5.5 mM and about 50 mM. Nanoparticle compositions including a therapeutic and/or prophylactic and a lipid component are prepared by combining the lipid solution with a solution including the therapeutic and/or prophylactic at lipid components to therapeutic and/or prophylactic wt:wt ratios between about 5:1 and about 50:1. The lipid solution is mixed with the nucleic acid solution, for example using a microfluidic or T-junction based system, at flow rates between about 0.5 ml/min and about 8 ml/min to produce a lipid nanoparticle suspension with a water to ethanol ratio between about 1:1 and about 4:1 preferably 3:1. Where the NP ratio (nitrogen to phosphate) is maintained between 4-7. The solution can be immediately diluted with buffer of choice or buffer exchanged as it is described below.

Examples of lipid nanoparticles that have been generated include:

TABLE 3
Lipid nanoparticle 1

| | | mol % of the total lipid |
|---|---|---|
| Lipid class | Lipid Type | present in the nanoparticle |
| cationic and/or ionisable | Dlin-MC3-DMA | 50% |
| lipid | | |
| phospholipid | DSPC | 10% |
| structural lipid | Cholesterol | 39.85%, |
| PEGylated lipid | DMG-PEG | 0.15% |

TABLE 4
Lipid nanoparticle 2

| | | mol % of the total lipid |
|---|---|---|
| Lipid class | Lipid Type | present in the nanoparticle |
| cationic and/or ionisable | Dlin-MC3-DMA | 50% |
| lipid | | |
| phospholipid | DSPC | 10% |
| structural lipid | Cholesterol | 39.85% |
| PEGylated lipid | DSPE-PEG | 0.15% |

TABLE 5
Lipid nanoparticle 3

| | | mol % of the total lipid |
|---|---|---|
| Lipid class | Lipid Type | present in the nanoparticle |
| cationic and/or ionisable | DODAP | 50% |
| lipid | | |
| phospholipid | DSPC | 10% |
| structural lipid | Cholesterol | 39.85% |
| PEGylated lipid | DSPE-PEG | 0.15% |

TABLE 6
Lipid nanoparticle 4

| | | mol % of the total lipid |
|---|---|---|
| Lipid class | Lipid Type | present in the nanoparticle |
| cationic and/or ionisable | DODAP | 50% |
| lipid | | |
| phospholipid | DSPC | 10% |
| structural lipid | Cholesterol | 39.85%, |
| PEGylated lipid | DMG-PEG | 0.15% |

For nanoparticle compositions including an RNA (such as those described herein including RBD-TM RNA), solutions of the RNA at concentrations of 0.133-0.6 mg/ml in deionized water are diluted in 25 mM sodium acetate buffer at a pH between 3 and 4.5 to form a stock solution (FIG. 1). After nanoparticle formulation, nanoparticle compositions can be processed by dialysis or tangential flow filtration to remove ethanol and achieve buffer exchange. For example, formulations are dialyzed twice against phosphate buffered saline (PBS), pH 7.4, at volumes 200 times that of the primary product using Slide-A-Lyzer cassettes (Thermo Fisher Scientific Inc., Rockford, Ill.) with a molecular weight cutoff of 10 kD. The first dialysis is carried out at room temperature for 3 hours. The formulations are then dialyzed overnight at 4° C. The resulting nanoparticle suspension is filtered through 0.4 or 0.22 μm sterile filters into glass vials and sealed. Nanoparticle composition solutions of 0.01 mg/ml to 0.50 mg/ml are generally obtained. The lipid nanoparticles used in the experiments described below were generated with DlinMC3 as the cationic and/or ionisable lipid.

Testing of the different lipid nanoparticle formulations revealed that formulations comprising between 0.1% mole to less than 1.5% mole PEGylated lipid resulted in greater update of the mRNA LNP into the spleen (as determined by spleen:liver ratio) and greater gene expression within the spleen. In particular, tissue targeting to the spleen following intramuscular injection was greatest when the percental mole of PEGylated lipid was between 0.1% mole to 0.5% mole. In these experiments, the amount of cholesterol in the LNP formulations was adjusted to account for the differing amounts of PEGylated lipid.

### Example 2: Inhibition of Binding of SARS-CoV-2 Spike Protein to hACE2

The percentage inhibition of ACE-RBD binding was determined. Mouse antiserum was incubated with RBD protein and added onto plates with ACE receptors. Plates washed and remaining RBD in the plate (bound to ACE) was determined. 100% inhibition indicates no binding of RBD was detected compared to full positive control, and mouse sera contains highly RBD-specific inhibiting antibodies. 0% inhibition indicates that the RBD fully bound to ACE and mouse antisera does not contain highly inhibiting antibodies for RBD:ACE binding (or Spike:ACE).

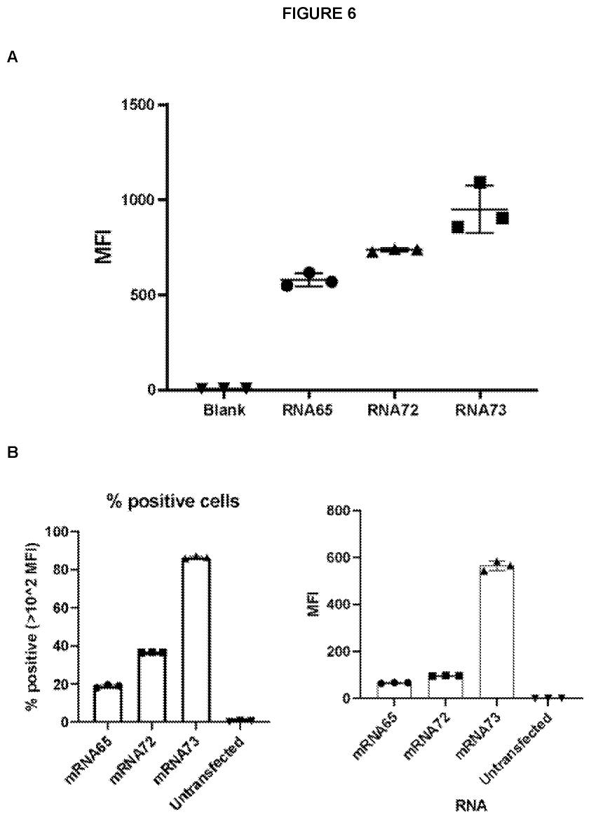

RBD-TM design shows superior neutralisation compared to other vaccine candidates, such as the whole S1 domain of the spike protein in either bound (S1-TM) or secreted (S1-Sec) form. FIG. 4 shows the percentage inhibition ACE:spike interaction for RBD-TM compared to S1-TM, S1-Sec, and controls.

### Example 4: Microneutralisation by RBD-TM

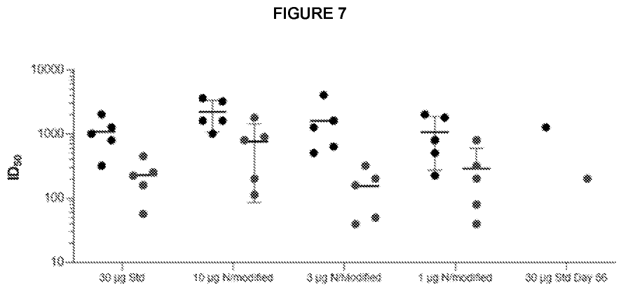

ID₅₀, the dilution of serum at which 50% of SARS-CoV-2 is neutralised in Vero cells, was determined. The results, presented in FIG. 5, demonstrate that RBD-TM provided superior neutralisation compared to bound (S1-TM) or secreted whole S1 domain (S1-Sec) from SARS-CoV-2.

### Example 5: Optimisation of RBD-TM Polynucleotide Sequence

The inventors investigated the effect of changes to the RBD-TM mRNA sequence. Two approaches were taken: i) depletion of uridine content and ii) conversion of serine-encoding codons to UCG.

The RNAs tested encode the same polypeptide sequence as encoded by the RNA used in Examples 1 to 4 (being encoded by SEQ ID NO: 2).

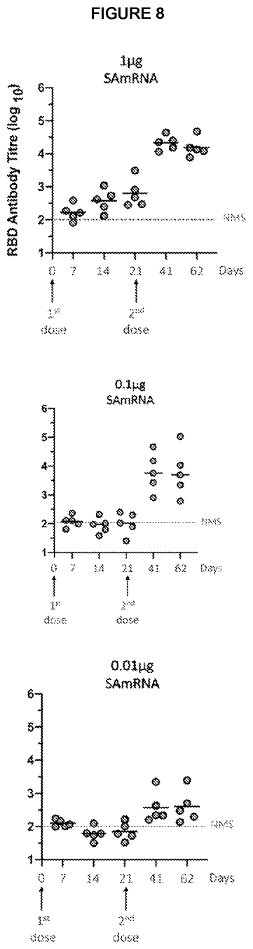

FIG. 6 shows the results of sequence optimisation in relation to improving levels of RBD-TM product at 24 hours (FIG. 6A) and improving the duration of RBD-TM protein (FIG. 6B). Results obtained using Flow-cytometer (BD Canto) and percentage and level of RBD expressing cells (Hela) were determined by transfecting the mRNA using lipid formulated mRNA 250 ng in 24 wells. 24 or 48 hours after transfection, cells were stained with anti-RBD/Anti-spike antibody followed by AF488. Flow cytometer settings were remained constant in the experiments. Mean fluorescence intensity and percentage of positive cells was calculated compared to transfected control (also called Blank) using FloJo software.

TABLE 7
Physicochemical properties of exemplified RNAs

| | | | | The free |
|---|---|---|---|---|
| | | | Mini- | energy of |
| | | | mum | the thermo- |
| | GC | Uridine | free | dynamic |
| | content | content | energy | ensemble |
| SEQ ID NO: 2 (“RNA65” - | 36% | 35% | −282.4 | −300.12 |
| non-optimised sequence) | | | | |
| SEQ ID NO: 10 (“RNA73” - | 61% | 17% | −400 | −414.09 |
| uridine depleted sequence) | | | | |
| SEQ ID NO: 11 (“RNA 72” - | 61% | 21% | −406.3 | −418.16 |
| uridine depleted and | | | | |
| conversion of serine- | | | | |
| encoding codons to UCG) | | | | |

Percentages of content represent approximate values (+/−1%). Free energies and thermodynamic ensemble were calculated using RNAfold server, and under default settings.

Table 7 above shows the different physicochemical properties of the sequences tested in the present example. The results indicate that reduction in uridine content and increased GC content facilitate increased levels of protein production and increased duration of protein production.

### Example 6: Efficacy of RBD-TM Vaccine Against SARS-CoV-2 Variants

An RBD-TM mRNA was produced comprising N-methyl pseudo-UTP (with novel flanking untranslated regions). The sequence comprised a codon-optimised version of the ancestral RBD-TM.

The ancestral (wild-type) RBD-TM induced anti-RBD antibody production after IM injection into mice. Serum samples obtained from inoculated mice were tested at day 42 (after prime on day 1 and boost on day 21). Serum samples are highly effective in neutralisation of viral infection of Vero cells.

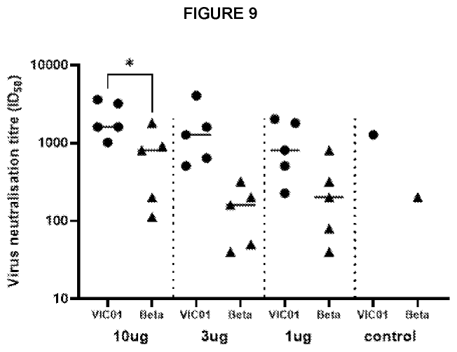

ID₅₀ is a measure of the extent to which the serum can be diluted but still neutralise the virus. ID₅₀ values greater than 100 are often protective against infection in an animal model. As shown in FIG. 7, the codon-optimised wild-type RBD-TM mRNA vaccine showed good activity at producing neutralising titres against both the wild-type and South African (Beta) variant strains, at doses as low as 1 μg in mice.

### Example 7: Development of RBD-Neutralising Antibodies after Prime and Boost IM Injection

An RBD-TM construct was generated using self-amplifying RNA, produced using native UTP using a replicon from the Venezuelan Equine Encephalitis (VEE) virus.

The VEE alphavirus replicon is well known and has been used to produce other mRNA vaccines. The replicon was formulated into a lipid nanoparticle and injected into mice.

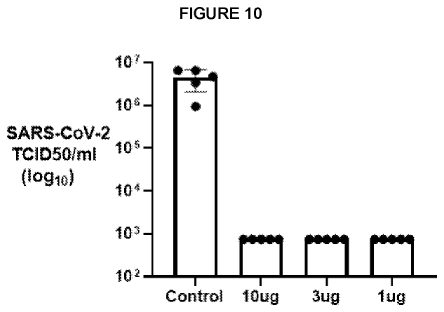

FIG. 8 shows antibody titres in mice following prime and boost IM injection of the self-amplifying RNA. Antibody titres of 10⁴ are indicative of strong activity. The formulation produced neutralising antibodies in mice at doses as low as 0.1 μg.

### Example 8: Immunogenicity and Protective Efficacy of the WT RBD-TM mRNA Vaccine in Mice

A WT RBD-TM mRNA vaccine (derived from “wildtype” SARS-Co-V2 sequence) was initially assessed in BALB/c mice where groups of 5 mice were inoculated with 1 μg, 3 μg or 10 μg of WT RBD-TM mRNA vaccine intramuscularly using a prime/boost regimen on Day 1 and 21.

### Immunogenicity

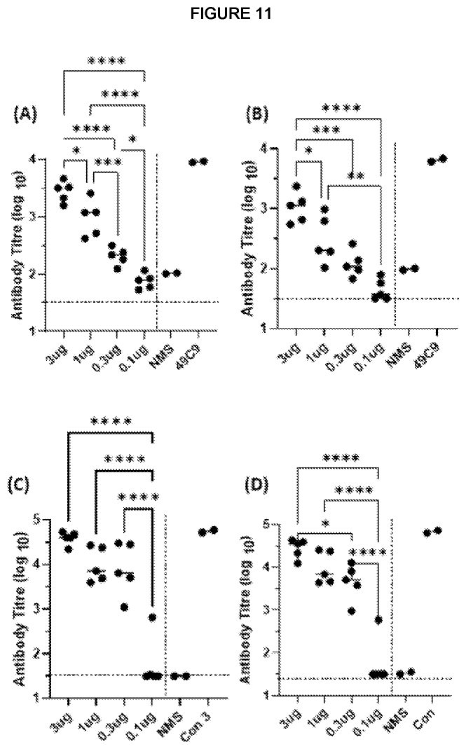

Sera collected on day 42 (21 days following the second immunisation) was assessed for meutralising antibodies (nABs) specific for both the WT (VIC01) and Beta (E1.351) SARS-CoV-2 strains. In general, all mice demonstrated higher levels of specific nABs capable of providing protection in the mammalian cell line against infection with the WT strain compared to the Beta SARS-CoV-2 strain (FIG. 9). Mice prime/boosted with the 10 μg dose of the WT RBD-TM mRNA vaccine appeared to have the highest nAB levels capable of protecting against both strains at Day 42 when compared to mice inoculated with the 3 μg and 1 μg dose.

### Protective Efficacy

Protective efficacy against lower airways (lung) infection was assessed using the Mouse SARS-CoV-2 challenge model where 44 days following the booster immunisation (Day 65) vaccinated and unvaccinated control mice were aerosol challenged with 10³ particles of the local N501Y SARS-CoV-2 isolate (hCoV-19/Australia/VIC2089/2020).

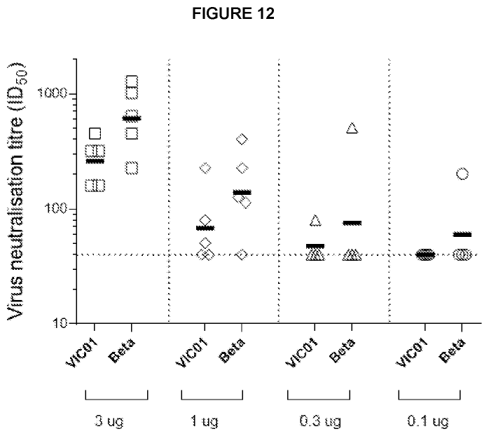

All 5 mice from each of the WT RBD-TM mRNA vaccinated groups demonstrated complete protection against the N501Y isolate of SARS-CoV-2 such that no detectable virus was observed in any of the lungs from vaccinated mice. Unvaccinated control mice challenged with the N501Y isolate of SARS-CoV-2 validated the challenge assay and high levels of virus were detected in the lungs of all 5 mice (FIG. 10).

### Example 9: Dose-Response Experiment Using BALB/c Mice Immunised with Beta Variant RBD-TM Vaccine in Prime/Boost Regime

The WT RBD-TM mRNA vaccine was adapted to produce a Beta vaccine, where the WT RBD-TM mRNA was substituted with the Beta variant RBD-TM sequence, which contained three mutations in the RBD (K417N/E484K/N501Y). The first dose-response study on the Beta RBD-TM vaccine in mice was carried out where groups of 5 BALB/c mice were immunised intramuscularly at doses of either 3 μg, 1 μg, 0.3 μg or 0.1 μg in a prime/boost regime on Days 0 and 21.

### Total Antibody Responses

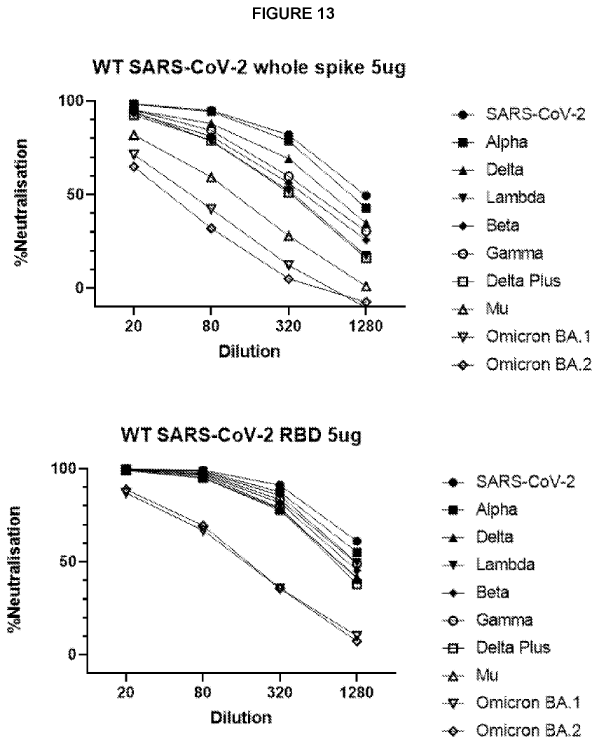

Total antibody responses specific for the WT RBD monomer and the Beta RBD monomer were assessed in sera collected from mice at Day 21 (after a single dose of mRNA vaccine as described herein) (FIG. 11A-B). Following two doses of the Beta RDB-TM mRNA vaccine, total antibody responses specific for the WT RBD monomer were assessed at days 42 and 56 (21 and 35 days post-secondary immunisation).

Twenty-one days following the primary immunisation, a linear dose-response trend was observed in sera collected from mice immunised with decreasing doses (from 3.0 μg and 0.1 μg) of the Beta RBD-TM vaccine. This trend was demonstrated in total antibody response to both the WT and Beta RBD monomers with higher antibody titres consistently observed against the Beta RBD monomer (11A) compared to the WT RBD monomer (FIG. 11B).

Total antibody titres specific for the WT RBD monomer were also analysed in the sera taken on day 42 and day 56 (21 and 35 days post-secondary immunisation) (11C-D)) where comparable antibody levels were observed on both days. On day 56, 35 days post-secondary immunisation, significantly higher WT RBD specific total antibody responses were observed in sera of mice immunised with 0.3 μg, 1 μg or 3 μg of the vaccine (4550, 9705, 27733 respectively) compared to those detected in the sera of mice immunised with 0.1 μg of the vaccine (57; p<0.0001). Mice immunised with 3 μg of mRNA-RBD-TM vaccine displayed the highest total antibody levels that was significantly higher than mice immunised with the 0.3 μg dose (27733 vs 4550: p<0.05). The control for panels A and B (49C9) was an antibody raised in mice that reacts with both wild-type RBD and Beta RBD. For panels C and D the control (Con) was pooled serum from previous neutralising doses of 30 μg native mRNA wild-type RBD-TM.

### Neutralising Antibody Responses

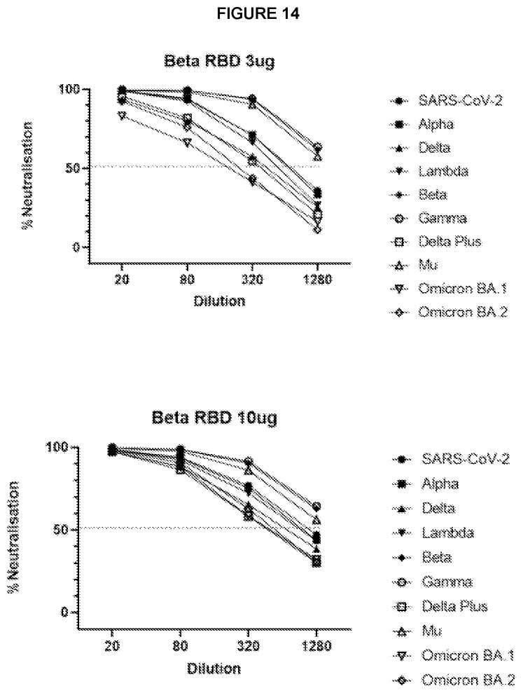

Sera collected on day 56 (35 days following the second immunisation) were assessed for nABs specific for both the VIC01 (an ancestral, “wild-type” isolate of SARS-CoV-2 that was established when the virus first entered Victoria, Australia) and Beta (B1.351) SARS-CoV-2 strains. All doses of the RBD-TM vaccine produced lower nABs titres specific for the wild-type variant compared to nAB levels specific for the Beta variant and a dose-response relationship was observed to both variants across the doses investigated (FIG. 12).

Mice prime/boosted with the 3 μg of the Beta vaccine indicated the highest ID₅₀ values (773 against Beta and 283 against VIC01) and all mice demonstrated neutralisation ID50 levels above 100. Results suggest the 3 μg dose provides omplete protection against the Beta SARS-CoV-2 and a high level of protection against the WT strain.

Levels of nAB in mice immunised with the intermediate 1 μg dose of Beta RBD-TM vaccine appeared lower than the 3 μg dose.

Mice prime/boosted with the lower doses, 0.1 μg and 0.3 μg, of the Beta RBD-TM vaccine demonstrated the lowest nAB levels.

A second dose-response study on the RBD-TM vaccine was conducted to (1) expand the dose range to 10 μg and omit the lowest 0.1 μg dose and (2) the test the reproducibility using a second independent batch of the vaccine.

In this second dose-response experiment groups of 4 or 5 BALB/c mice were immunised with either 10 μg, 3 μg, 1 μg or 0.3 μg of the vaccine in a prime/boost regime on Days 0 and 21. Sera were collected on day 56 to assess total and nAB responses.

Total antibody titres specific for the WT RBD monomer were analysed in the sera of mice immunised with in these vaccinated mice on day 56 (35 days post-secondary immunisation). Results (not shown) were consistent with the first dose-response experiment and suggested that the total antibody response to a 10 μg dose was not higher than a 3 μg dose.

Neutralising antibody responses specific for both the WT (VIC01) and Beta (B1.351) SARS-CoV-2 variants were also assessed. Results demonstrated the 10 μg, 3 μg and 1 μg dose of the RBD-TM vaccine (comprising the beta RBD sequence) produced lower nABs titres specific for the WT variant compared to nAB levels specific for the Beta variant. However, a reverse trend was observed for the lowest 0.3 μg dose where majority of individual responses were below a neutralisation ID50 of 100.

Consistent with previous results, the ability of nABs to neutralise mammalian cell infection of the WT and Beta virus was again observed in mice immunised with the 3 μg dose of vaccine. Mice immunised with the 10 μg of vaccine did not demonstrate any further increase in nAB levels against either variant, where neutralisation ID₅₀ levels were comparable to those indicated from mice immunised with the 3 μg dose.

These results suggest the 3 μg dose is the optimal dose for immunogenicity of the RBD-TM vaccine.

### Example 10: Activity of Beta Variant RBD-TM Vaccine Against Various Strains of SARS-CoV-2

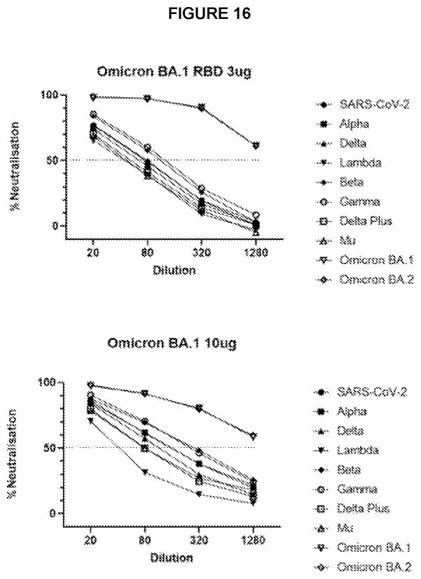

A surrogate SARS-CoV-2 neutralisation test was used which determines antibody-mediated blockage of ACE2-spike protein-protein interaction. This allows serum to be tested for its ability to block binding of different SARS-CoV-2 variants. FIG. 14 shows that the Beta vaccine generates antibodies that bind strongly to Beta as well as other variants including the Omicron BA.1 variant, including to the Alpha, Delta, Delta plus, Lambda, Gamma, Mu variants.

### Example 11: Activity of Omicron BA.1 Variant RBD-TM Vaccines Against Various Strains of SARS-CoV-2

In this experiment naïve Balb/c mice were vaccinated using a BA.1 variant using the usual regimen of vaccination on days 1 and 21, using doses of either 0, 1, 3 or 10 ug mRNA formulated in LNP, as described herein. Sera collected at day 56 were tested as above using the in vitro microneutralisation test described above to identify whether antibodies induced could protect Vero cells from infection by either the wild type, Beta or Omicron BA.1 variants of SARS-CoV-2.

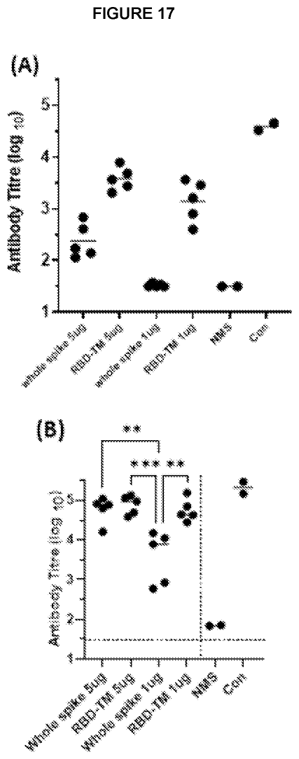

Doses of 3 or 10 ug BA.1 RBD-TM vaccine induced antibodies that gave very good protection against BA.1 but little or no protection against wild-type or Beta (FIG. 15). This illustrates the marked differences between Omicron and the other variants and also demonstrates that the BA1 RBD-TM can induce strong specific responses against Omicron.

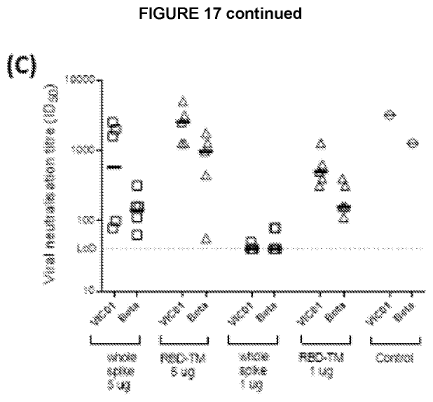

A surrogate SARS-CoV-2 neutralisation test was used which determines antibody-mediated blockage of ACE2-spike protein-protein interaction. This allows serum to be tested for its ability to block binding of different SARS-CoV-2 variants. FIG. 16 shows that the Omicron BA.1 vaccine generates antibodies that bind strongly to Omicron BA.1 but these have limited activity against other variants.

### Example 12: Description of Exemplary Drug Product

Description of the mRNA

The RNA component of the RNA-LNP drug product is nucleoside modified mRNA (modRNA). The modRNA encodes for an engineered form of the SARS-CoV-2 spike protein, which includes the RBD linked to the TM domain and the ‘intracellular’ CT domain. This construct is referred to herein as RBD-TM. Exemplary sequences of RBD-TM mRNA sequences, and corresponding translated protein product are provided in Table 1.

The mRNA drug substance is a single-stranded, 5-capped mRNA that is translated upon entering the cell. In addition to the sequence encoding the viral receptor binding domain, transmembrane and CT domains (RBD-TM) of the SARS-CoV-2 spike protein (i.e., open reading frame), the mRNA drug substance contains structural elements optimized for high efficacy of the RNA:
inhouse designed untranslated regions (5′ and 3′ UTRs);
PolyA-125 bases (Poly (A) tail);
Cap1, and
all the uridines are replaced with N-methyl-pseudouridine

Capping was performed using the commercial capping reagent Cap1 from TriLink Biotechnologies Inc. Other capping reagents may be used, including but not limited to Cap 0 and Cap 2.

The drug product is highly purified single-stranded, 5-capped mRNAs formulated in LNP in aqueous cryoprotectant buffer for intramuscular (IM) administration. The formulated RBD-TM mRNA is preservative-free and presented as a clear/cloudy liquid for intramuscular injection and contains highly purified single-stranded, 5-capped mRNA formulated with LNPs suspended in a sterile isotonic tromethamine/sucrose buffer solution (pH 7.2-7.4).

The RNA drug substance is the only active ingredient in the drug product.

The composition of an exemplary RBD-TM vaccine is given in Table 8.

TABLE 8
Composition of an exemplary RBD-TM vaccine

| Component | Function |
|---|---|
| Drug Substance (mRNA) | Active substance |
| DLin-MC3-DMA | Functional Ionisable lipid |
| [(6Z,9Z,28Z,31Z)-heptatriaconta- | |
| 6,9,28,31 tetraen-19-yl-4-(dimethylamino) | |
| butanoate] (50 mole %) | |
| DMG-PEG 2000 | Functional PEGylated Lipid |
| [Polyethylene glycol [PEG] | |
| 2000 dimyristoyl glycerol] (0.2 mole %) | |
| DSPC | Structural ‘helper’ Lipid |
| [Distearoylphophatidylcholine] (10 mole %) | |
| Cholesterol (39.8 mole %) | Structural ‘helper’ Lipid |

The above LNP formulation is suspended in 25 mM Tris buffer (pH 7.4) with 8.8% w/w sucrose as a cryoprotectant.

### Description of Excipients

Independently of the variant SARS-CoV-2 target, exemplary RED-TM vaccines of the invention contain the four lipids: DLin-MC3-DMA (from DC Chemicals, Catalog #DC10800); Cholesterol (plant-derived, from Sigma (Merck), catalog #C1231-1G), DSPC (also referred to as 18:0 PC; from Avanti Polar, catalog #850365P-1g) and DMG-PEG 2000 (from Avanti Polar, catalog #880151P-1g) at a mole percentage ratio of 50:39.8:10:0.2. In addition to the testing documented in the certificate of analysis the identity and purity of each of the lipid materials supplied was validated by nuclear magnetic resonance (NMR), mass spectrometry (MS) and liquid chromatography-mass spectrometry (LCMS).

The structures of the four lipids are listed in Table 9.

TABLE 9
The structure of the four lipids

| | Molecu- | |
|---|---|---|
| | lar | |
| Excipient | Weight | Structure |
| DLin-MC3-DMA [(6Z,9Z,28Z,31Z)- heptatriaconta- 6,9,28,31 tetraen- 19-yl-4- (dimethylamino) butanoate] | 642.09 | 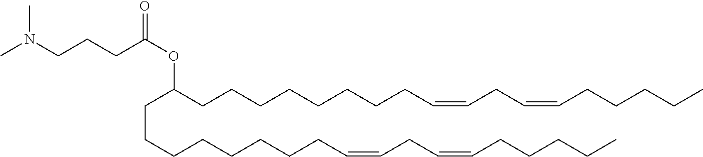 [SMILES: CCCCC/C=C\C/C=C\CCCCCCCCC(CCCCCCCC/C=C\C/C=C\CCCCC)OC(=O)CCCN(C)C] |
| DMG-PEG 2000 [Polyethylene glycol [PEG] 2000 dimyristoyl glycerol] | 2552.133 | 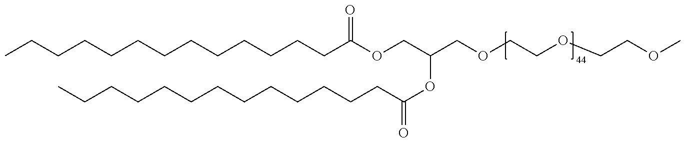 |
| DSPC [Distearoyl- phophatidyl choline] | 790.145 | 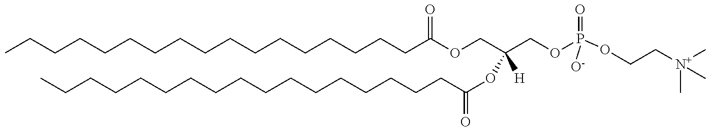 [SMILES: CCCCCCCCCCCCCCCCCC(=O)OC[C@H](COP(=O)([O-])OCC[N+](C)(C)C)OC(=O)CCCCCCCCCCCCCCCCC] |
| Cholesterol | 386.65 | 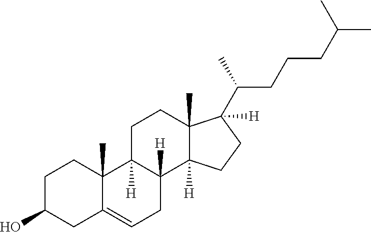 |

### Manufacture and Quality Control of the Vaccine

Manufacture of the vaccine using the purified RNA is represented by the following steps. An aqueous solution of mRNA at pH 4 was mixed with a solution of the four lipids in ethanol, using a microfluidics mixing device (eg. NxGen Ignite or Blaze Nanoassemblr) supplied by Precision Nanosystems. The rapid, controlled, homogenous mixing produces homogeneous nanoparticles. The suspension of nanoparticles was adjusted to a pH of 7.4 simultaneously with removal of ethanol using dialysis or tangential flow filtration, then adjustment with sucrose solution to produce the final form of the product. The particles were then sterile filtered (0.22 μm) prior to being filled into sterile vials. Characterization of the RNA LNP included analysis for RNA content, RNA integrity, lipid content, and endotoxin content. Consistency of the Particle size was determined by dynamic light scattering, a standard method for submicron dispersions, using a Zetasizer (Malvern Instruments).

Potency of each RBD-TM vaccine was assessed by transfection of cultured HeLa cells, simultaneously with LNPs loaded with mRNA encoding nanoluciferase as an internal standard. The expression of RBD-TM at the cell surface was quantified by immunofluorescence-flow cytometry using a monoclonal anti-Beta RBD primary antibody, and by immunoluminescence using a plate reader. The two assays of RBD-expression by HeLa cells were calibrated by transfecting cells with a freshly prepared standard LNP-mRNA over a range of doses to produce a dose-expression plot. This allowed a semi-quantitative means of comparing the potency of laboratory batches, the toxicity batch and products for clinical use.

### Example 13: Comparison of WT RBD-TM mRNA Vaccine with WT Spike Protein mRNA Vaccine Using an Exemplary LNP Formulation

Three pre-clinical experiments were conducted to compare the vaccine of the previous examples (herein “MIPs vaccine”) with that of a WT Spike protein mRNA vaccine using the same sequence as reported for use in the Pfizer's Comirnaty^{T}M 30 μg intramuscular mRNA vaccine. The Comirnaty™ mRNA sequence was obtained from a WHO document published as part of its International non-proprietary names programme (World Health Organisation, 2020). Comparisons were also made using the RBD-TM and whole spike protein sequences in either the MIPS LNP formulation (Table 8) or the LNP formulated to align with that reported as used in the Comirnaty™ vaccine. The formulation of Comirnaty™ can be found in the FDA product factsheet.

The three step pre-clinical assessment included the following vaccine formulations:
1) Comparing the WT RBD-TM mRNA vaccine with WT Spike protein mRNA (using the reported sequence in the Comirnaty™ vaccine), both formulated in the MIPS LNP formulation (eg per Table 8);
2) Comparing the WT RBD-TM mRNA and WT Spike protein mRNA sequences formulated in a lipid formula based on that reported as used in Comirnaty™;
3) Finally, the RBD-TM mRNA (derived from beta variant) was formulated with four LNP formulations using the specific lipids and lipid ratios from either Table 8 or alternative LNP formulations for comparison.
   Comparison 1: Comparison of WT RBD-TM mRNA Vaccine with WT Spike Protein mRNA Vaccine Using the MIPS LNP Formulation.

A comparison experiment was conducted to compare a WT RBD-TM mRNA vaccine with the WT whole spike protein mRNA vaccine using either of two doses of mRNA in an LNP formulation. The whole spike protein mRNA sequence was based on the sequence used in the Comirnaty™ vaccine.

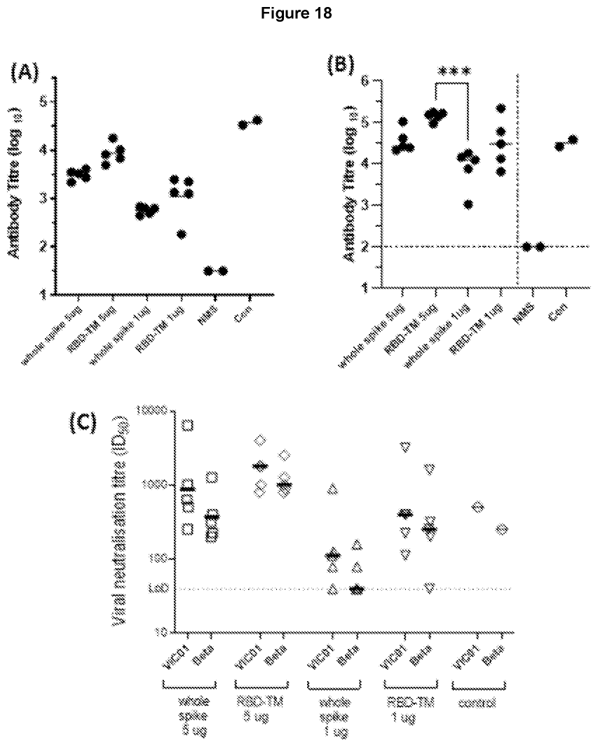

Groups of 5 BALB/c mice were vaccinated intramuscularly with either whole WT spike protein mRNA vaccine or WT RBD-TM mRNA vaccine in an LNP formulation as described in Table 8; and at doses of 1 μg or 5 μg in a prime boost regime on days 0 and 21. Sera were used to assess and compare total antibody responses (on day 21 and day 42) and neutralisation antibody responses (Day 42 only) (FIG. 17).

Results indicated that the WT RBD-TM mRNA vaccine was more immunogenic when compared to a WT whole spike protein mRNA vaccine in both primary (FIG. 17A) and secondary (FIG. 17B) antibody responses. Following the booster immunisation on Day 42, total antibody levels specific for the WT RBD monomer were significantly higher in mice administered with a prime/boost of the 1 μg (56364, p<0.01) or 5 μg (77446, p<0.001) dose of WT RBD-TM mRNA vaccine and 5 μg dose of WT whole spike protein mRNA (59620, p<0.01) compared to 1 μg of the WT whole spike protein mRNA (3690) (FIG. 17B).

Sera collected on day 42 (21 days after the secondary immunisation) were also used to assess and compare nAB responses using the mNT assay with both the wild-type (WT) (as represented by VIC01—an ancestral isolate of SARS-CoV-2 that was established when the virus first entered Victoria, Australia) and Beta virus strains. Trends observed in the nAB responses correlated with total antibody responses, where 1 μg of the WT RBD-TM mRNA vaccine had similar vaccine potency to 5 μg of the WT whole spike protein mRNA vaccine. Both WT mRNA vaccines were less effective in neutralising the Beta variant. The subsequent nAB titres against the WT strain were higher than against the Beta strain in all groups (FIG. 17).

Comparison 2: Comparison of WT RBD-TM mRNA and WT Spike Protein mRNA Using an Alternative LNP Formulation

To compare the LNP formulation of Table 8 with an alternative LNP formulation based on the Comirnaty™ vaccine, a second LNP formula was generated using 46.3% ALC-0315 (lonisable lipid); 9.4% DSPC and 42.7% cholesterol (helper lipids) and 1.6% DMG-PEG 2000 (PEGylated lipid).

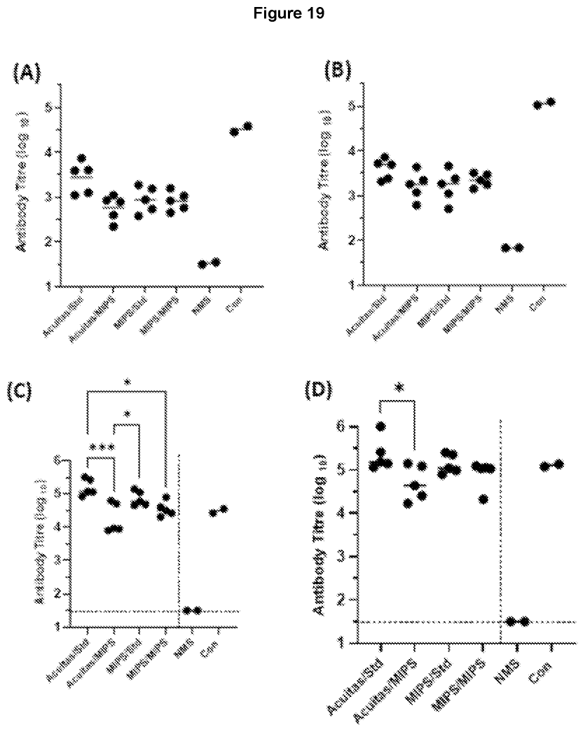

Groups of 5 BALB/c mice were immunised with (1) WT whole Spike Protein mRNA or (2) WT RBD-TM mRNA in the alternative LNP formulation at doses of 1 μg or 5 μg in a prime boost regime on days 0 and 21. Sera were used to assess and compare total antibody responses (on day 21 and day 42) and neutralisation antibody responses (Day 42 only) (FIG. 18).

Results demonstrated a trend for primary antibody responses to the WT RBD-TM mRNA to be slightly higher than to the WT whole spike protein mRNA when incorporated with the alternative LNP formulation (FIG. 18A). After two doses of each vaccine both the antibody titres and viral neutralisation titres in either the first formulation (FIG. 17) or alternative formulation (FIG. 18) adopted a similar pattern of activity, suggesting that at either the 1 μg or 5 μg dose, the RBD-TM vaccine was more active than the whole spike vaccine.

Comparison 3: Comparison of the Beta RBD-TM mRNA with Four Different LNP Formulations Based on the Specific Lipids and Lipid Ratios from Either the Table 8 Formulation (“MIPs Formulation”) or Alternative LNP Formulation

In a related set of experiments, vaccines were generated using Beta RBD-TM mRNA with four different LNP formulations based on the specific lipids and lipid ratios from either the formulation of Table 8 or alternative LNP formulation.

Inspection of the antibody titres and neutralisation titres shown in FIGS. 17 and 18 suggests that the formulation of Table 8 and alternative formulation are not sufficiently different to warrant adjustment of the dose of mRNA to correct for differences in formulation. To compare the effects of the choice of lipids and the molar ratios in the formulation, a direct comparison of the activities of the Beta RBD-TM in four different LNP formulations was conducted. Five BALB/c mice were vaccinated with 3 μg doses of Beta RBD-TM, on days 1 and 21, and sera were collected at day 21 and 42. These mice were later challenged by infection with Beta variant SARS-CoV-2. The four salient formulations are described, using the notation “lipids”/“formula”, as outlined in Table 10 below and:
1) the lipids and LNP formulation based on the Comirnaty™ vaccine (as in Comparison 2);
2) lipids used in Comirnaty™ (as in Comparison 2) but at the ratios used in Table 8 herein;
3) lipids in table 8 but in the ratios used in Comirnaty™ (ie in Comparison 2); and
4) formulation of Table 8 (ie MIPS lipids at MIPS lipid ratio).

| | | MIPS | | Alternative |
|---|---|---|---|---|
| | MIPS | Lipid ratio | Alternative | Lipid ratio |
| Lipid | Lipids | (%) | Lipids | (%) |
| Functional | DLin-MC3- | 50 | ALC-0315 | 46.3 |
| Ionisable | DMA | | | |
| lipid | | | | |
| Functional | DMG-PEG | 0.2 | DMG-PEG | 1.6 |
| PEGylated | 2000 | | 2000 | |
| Lipid | | | | |
| Structural | DSPC | 10 | DSPC | 9.4 |
| ‘helper’ Lipid | | | | |
| Structural | Cholesterol | 39.8 | Cholesterol | 42.7 |
| ‘helper’ Lipid | | | | |
| Cholesterol | | | | |

The antibody titres at day 42 were high for all formulations against both WT RBD and Beta RBD-coated plates. Titres were slightly higher against Beta RBD-coated plates. The four formulations were effectively bioequivalent with respect to viral neutralisation and were more active at neutralising infection by the Beta variant of SARS-CoV-2. It can be concluded that the formulation used in Table 8 was bioequivalent to the alternative formulation based on that used in Comirnaty™.

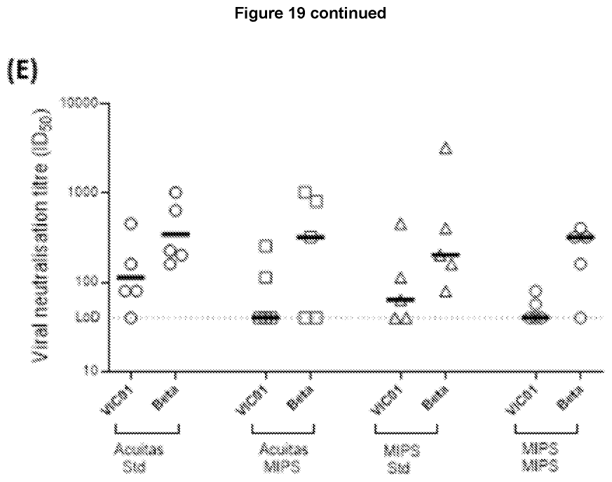

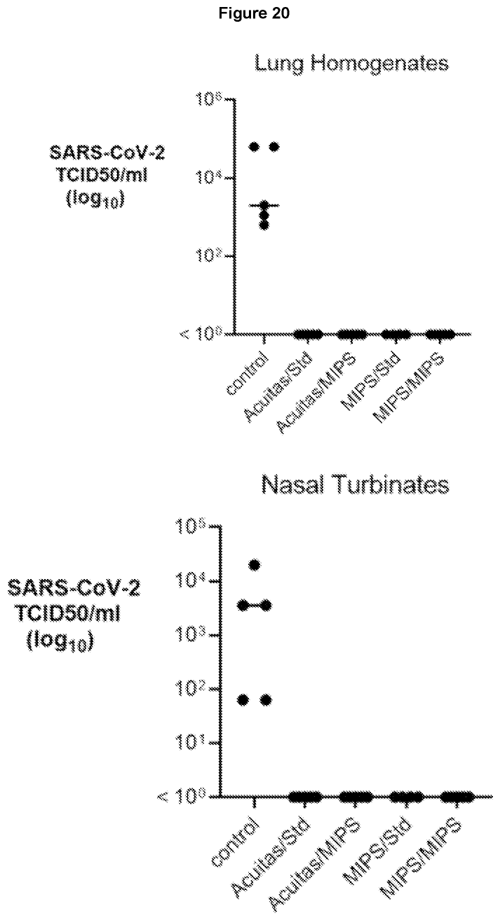

The mice that participated in the experiments shown in FIG. 19 were challenged by infection with the Beta variant of SARS-CoV-2. Five unvaccinated BALB/c mice of equivalent age were used as controls. The data is shown in FIG. 20. On day 65 mice were infected with 10^{4.5} infectious units of SARS-CoV-2 Beta variant. At the expected peak of infection (three days after the virus inoculation), the vaccinated mice did not have any infectious virus detected in either the nasal turbinates, or the lungs via a 50% tissue culture infectious dose assay (TCID₅₀). Meanwhile, the unvaccinated mice exhibited an average viral burden of 10³ TCID₅₀/mL infectious SARS-CoV-2 in the nasal turbinates and 10^{3.75} TCID⁵⁰/mL in the lungs. These results indicate that vaccination with each of the four formulations enabled mice to develop an immune memory that effectively prevented infection by the SARS-CoV-2 Beta variant of concern.

The conclusion based on data from these experiments, is that the immune responses to the MIPS RBD-TM vaccine are likely to be 3-5 times more potent per unit mass than whole spike vaccines, and that the formulation used performs with similar efficiency as an alternative formulation based on that used in in Comirnaty™. It is also noteworthy that the dose range of 1-5 μg, which spans the range of moderate to high immune responses to the RBD-TM vaccine, correlates well with mouse data from studies of the BionTech/Pfizer and Moderna mRNA vaccines.

It will be understood that the invention disclosed and defined in this specification extends to all alternative combinations of two or more of the individual features mentioned or evident from the text or drawings. All of these different combinations constitute various alternative aspects of the invention.

1. A polynucleotide encoding a chimeric or fusion protein comprising or consisting of:
a) an N-terminal secretion signal peptide;
b) an amino acid sequence of the receptor binding domain (RBD) of a spike protein of a coronavirus, or variant thereof; and
c) a C-terminal domain comprising a transmembrane region and a cytoplasmic region
preferably, wherein the polynucleotide is capable of being translated in a mammalian cell.

2. The polynucleotide of claim 1, wherein the polynucleotide is a messenger RNA (mRNA) molecule.

3. The polynucleotide of claim 2, wherein the mRNA further comprises one or more selected from: a 5′ untranslated region (UTR), a 3′ UTR, a 5′ cap analog and a polyadenine (polyA) tail.

4. The polynucleotide of claim 2 or 3, wherein the mRNA comprises a chemical modification.

5. The polynucleotide of any one of claims 2 to 4, wherein the mRNA is codon optimised, preferably wherein the codons encoding serine residues are comprise the nucleotide sequence UCG.

6. The polynucleotide of any one of claims 3 to 5, wherein the mRNA is depleted of uridine nucleosides relative to the viral polynucleotide sequence encoding the RBD; optionally wherein at least 25%, at least 30%, at least 35%, at leat 40%, at least 45%, at least 50%, at least 55%, at least 60%, at least 65%, at least 70%, at least 75%, at least 80%, at least 85%, at least 90%, at least 95%, or where 100% of the uridine nucleosides are replaced with N-methyl-pseudouridine.

7. The polynucleotide of any one of claims 1 to 6, wherein the coronavirus is a beta-coronavirus, preferably a beta-coronavirus from Lineage B, such as SARS-CoV or SARS-CoV-2.

8. The polynucleotide of any one of claims 1 to 6, wherein coronavirus is a beta-coronavirus from Lineage C, such as MERS-CoV.

9. The polynucleotide of any one of claims 1 to 6, wherein the coronavirus is SARS-CoV-2 or a mutated form or variant thereof, such as the alpha, beta, epsilon, kappa, delta, delta-plus or lambda variants.

10. The polynucleotide of claim 9, wherein the amino acid sequence of the RBD is set forth in SEQ ID NO: 9, or is a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity thereto.

11. The polynucleotide of claim 10 wherein the RBD has a variation of mutation selected from one or more of N191, E174, K107, L142, T168 or S167 of SEQ ID NO: 9 (equivalent to residues N501, E484, K417, L452, T478, K417 or S447 of SEQ ID NO: 1).

12. The polynucleotide of any one of claims 1 to 11, wherein the N-terminal secretion signal peptide comprises any amino acid sequence which enables the chimeric or fusion protein to be processed by ribosomes bound to the rough endoplasmic reticulum (ER) of a cell, and thereby results in threading of the chimeric or fusion protein into the ER.

13. The polynucleotide of claim 12, wherein the N-terminal secretion signal peptide comprises the amino acid sequence of any secretion signal from a coronavirus, preferably, from a coronavirus spike protein.

14. The polynucleotide of any one of claims 1 to 13, wherein the N-terminal secretion signal peptide comprises an amino acid sequence that is cleavable to enable cleavage of the RBD amino acid sequence from the secretion signal peptide following translation of the polynucleotide sequence.

15. The polynucleotide of any one of claims 1 to 14, wherein the signal peptide comprises the sequence as set forth in SEQ ID NO: 4, or a sequence at least 80%, at least 85%, at least 90%, at least 95%, at least 96%, at least 97%, at least 98% or at least 99% identical thereto.

16. The polynucleotide of any one of claims 2 to 15, wherein the C-terminal domain comprises the amino acid sequence of a transmembrane domain from a coronavirus spike protein.

17. The polynucleotide of claim 16, wherein the C-terminal domain comprises the amino acid sequence of the transmembrane domain of the SARS-CoV-2 spike protein.

18. The polynucleotide of any one of claims 7 to 17, wherein the C-terminal domain comprises a transmembrane domain comprising the amino acid sequence as set forth in SEQ ID NO: 5 or 6, or a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto.

19. The polynucleotide of any one of claims 1 to 18, wherein the C-terminal domain comprises the amino acid sequence of the cytoplasmic region of a coronavirus spike protein.

20. The polynucleotide of claim 19, wherein the C-terminal domain comprises the amino acid sequence of the SARS-CoV-2 spike protein.

21. The polynucleotide of any one of claims 7 to 20, wherein the C-terminal domain comprises a cytoplasmic region comprising the amino acid sequence as set forth in SEQ ID NO: 7, or a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto.

22. The polynucleotide of any one of claims 7 to 21, wherein the C-terminal domain comprises the amino acid sequence as set forth in SEQ ID NO: 8, or a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto.

23. The polynucleotide of any one of claims 1 to 22, wherein the polynucleotide encodes a chimeric or fusion protein comprising or consisting of the sequence set forth in any one of SEQ ID NOs: 3, 12, 15, 18, 21, 24 or 27 or a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto.

24. The polynucleotide of any one of claims 1 to 23, wherein the polynucleotide comprises or consists of the nucleic acid sequence set forth in any one of SEQ ID NOs: 2, 10, 11, 13, 16, 19, 22, 25 or 28 or a sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto.

25. The polynucleotide of any one of claims 1 to 24, wherein the chimeric or fusion protein comprises a flexible linker sequence between b) the amino acid sequence of the RBD and c) the C-terminal domain.

26. The polynucleotide of claim 25, wherein the linker comprise a series of amino acid residues, such as glycine, serine, glutamic acid or aspartic acid residues, which impart flexibility to the polypeptide.

27. The polynucleotide of any one of claims 1 to 26, wherein the polynucleotide does not encode a full-length S1 domain of the spike protein of a coronavirus.

28. The polynucleotide of any one of claims 1 to 26, wherein the polynucleotide does not encode any of the region of a coronavirus spike protein N-terminal to the RBD in the naturally occurring S1 domain.

29. The polynucleotide of any one of claims 1 to 26, wherein the polynucleotide further comprises a sequence encoding an amino acid sequence of a coronavirus spike protein N-terminal domain.

30. The polynucleotide of any one of claims 1 to 29, wherein the polynucleotide, preferably an mRNA, is formulated in a lipid nanoparticle, lipsome, lipid vesicle, or lipoplex.

31. A composition comprising a polynucleotide of any one of claims 1 to 30 and a lipid component.

32. The composition of claim 31, wherein the composition is in the form of a liposome, lipid vesicle, lipoplex (such as a lipid-polycation complex), or lipid nanoparticle.

33. The composition of claim 32, wherein the composition is a lipid nanoparticle.

34. The composition of claim 33, wherein the lipid nanoparticle comprises a cationic and/or ionisable lipid, a phospholipid, a PEG lipid, and a structural lipid.

35. The composition of claim 34, wherein the lipid nanoparticle comprises 20-60% ionizable cationic lipid, 5-25% neutral lipid, 25-55% cholesterol, and 0.5-15% PEG-modified lipid.

36. The composition of claim 34, wherein the lipid nanoparticle comprises
a cationic and/or ionisable lipid comprising from about 40 mol % to about 60 mol % of the total lipid present in the nanoparticle;
a phospholipid comprising from about 5 mol % to about 20 mol % of the total lipid present in the nanoparticle;
a structural lipid comprising from about 30 mol % to about 60 mol % of the total lipid present in the nanoparticle; and/or
a PEGylated lipid comprising from about 0.05 mol % to less than 0.5 mol % of the total lipid present in the nanoparticle.

37. The composition of any one of claims 33 to 36, wherein the lipid nanoparticle is between about 50-500 nm in diameter.

38. The composition of any one of claims 33 to 37, wherein the nanoparticle has a negative, positive or neutral charge.

39. The composition of any one of claims 33 to 37, wherein the nanoparticle has a diameter of at least about 100 nm or greater, and has a negative charge.

40. The composition of any one of claims 36 to 39, wherein the ionisable lipid comprises DLin-MC3-DMA [(6Z,9Z,28Z,31Z)-heptatriaconta-6,9,28,31 tetraen-19-yl-4-(dimethylamino) butanoate] or ALC-0315.

41. The composition of any one of claims 36 to 40, wherein the PEGylated lipid comprises Polyethylene glycol [PEG]2000 dimyristoyl glycerol.

42. The composition of any one of claims 36 to 41, wherein the structural lipid comprises one or both of cholesterol and distearoylphophatidylcholine.

43. The composition of any one fo claims 36 to 42, wherein the composition further comprises a cryopreservative, optionally in the form of sucrose or other sugar.

44. A method for producing a lipid nanoparticle comprising a polynucleotide of any one of claims 1 to 29, the method comprises formulating any polynucleotide of the invention, with a mixture of lipids, optionally wherein the mixture comprises a cationic lipid, neutral lipid, cholesterol and a PEGylated lipid.

45. The method of claim 44, wherein the mixture of lipids is as defined in any one of claims 23 to 43.

46. A nucleic acid construct or vector, comprising a polynucleotide of any one of claims 1 to 29.

47. The vector of claim 46, wherein the vector is suitable for production of mRNA from a DNA template.

48. A polypeptide produced or synthesised from the polynucleotide of any one of claims 1 to 29.

49. A method for eliciting an immune response to a coronavirus in a subject in need thereof, the method comprising administering to the subject, a polynucleotide of any one of claims 1 to 30, a composition of any one of claims 31 to 43 or a nucleic acid or vector of claim 46 or 47.

50. A method for eliciting an immune response to a coronavirus in a subject in need thereof, the method comprising administering to the subject, a nanoparticle composition comprising:
i) a lipid component; and
ii) a polynucleotide of any one of claims 1 to 29, wherein the polynucleotide is capable of being translated in the cell to produce the polypeptide encoded by the polynucleotide.

51. A method for producing an RBD from a coronavirus spike protein in a mammalian cell, the method comprising contacting the mammalian cell with a nanoparticle composition, the composition comprising:
i) a lipid component; and
ii) a polynucleotide of any one of claims 1 to 29, wherein the polynucleotide is capable of being translated in the cell to produce the RBD.

52. The method of claim 50 or 51, wherein the lipid component comprises a phospholipid, a PEG lipid, and a structural lipid.

53. The method of claim 52, wherein the lipid component comprises:
a cationic and/or ionisable lipid comprising from about 40 mol % to about 60 mol % of the total lipid present in the nanoparticle;
a phospholipid comprising from about 5 mol % to about 20 mol % of the total lipid present in the nanoparticle;
a structural lipid comprising from about 30 mol % to about 60 mol % of the total lipid present in the nanoparticle; and/or
a PEGylated lipid comprising from about 0.05 mol % to less than 0.5 mol % of the total lipid present in the nanoparticle.

54. The method of claim 52 or 53, wherein the lipid component comprises:
an ionisable lipid that comprises DLin-MC3-DMA [(6Z,9Z,28Z,31Z)-heptatriaconta-6,9,28,31 tetraen-19-yl-4-(dimethylamino) butanoate] or ALC-0315;
a PEGylated lipid that comprises Polyethylene glycol [PEG]2000 dimyristoyl glycerol; and/or
a structural lipid that comprises one or both of cholesterol and distearoylphophatidylcholine.

55. Use of a polynucleotide of any one of claims 1 to 30, a nucleic acid or vector of claims 46 or 47, in the manufacture of a composition for eliciting an immune response to a coronavirus in a subject.

56. A polynucleotide of any one of claims 1 to 30, or a nucleic acid or vector of claims 46 or 47, or a composition of any one of claims 31 to 43, for use in eliciting an immune response to a coronavirus in a subject.


SEQUENCE LISTING (29 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - RNA, 990 nt, synthetic construct
  source 1..990: mol_type = other RNA
atgtttgtttttcttgttttattgccactagtctctagtcagtgtgttaatcttacaaccggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtggttcctccggtggttatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataatga

SEQ ID NO: 3 - protein, 328 aa, synthetic construct
MFVFLVLLPLVSSQCVNLTTGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGGSSGGYEQYIKWPWYIWLGFIAGLIAIVMVTIMLCCMTSCCSCLKGCCSCGSCCKFDEDDSEPVLKGVKLHYT

SEQ ID NO: 4 - protein, 20 aa, SARS-CoV-2
MFVFLVLLPLVSSQCVNLTT

SEQ ID NO: 5 - protein, 34 aa, SARS-CoV-2
WPWYIWLGFIAGLIAIVMVTIMLCCMTSCCSCLK

SEQ ID NO: 6 - protein, 26 aa, SARS-CoV-2
YEQWPWYIWLGFIAGLIAIVMVTIML

SEQ ID NO: 7 - protein, 28 aa, SARS-CoV-2
GCCSCGSCCKFDEDDSEPVLKGVKLHYT

SEQ ID NO: 8 - protein, 68 aa, SARS-CoV-2
YEQYIKWPWYIWLGFIAGLIAIVMVTIMLCCMTSCCSCLKGCCSCGSCCKFDEDDSEPVLKGVKLHYT

SEQ ID NO: 9 - protein, 234 aa, SARS-CoV-2
GIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFN

SEQ ID NO: 10 - RNA, 990 nt, synthetic construct
  source 1..990: mol_type = other RNA
atgttcgtgttcctggtgctgctgcccctggtgagcagccagtgcgtgaacctgaccaccggcatctaccagaccagcaacttcagggtgcagcccaccgagagcatcgtgaggttccccaacatcaccaacctgtgccccttcggcgaggtgttcaacgccaccaggttcgcgagcgtgtacgcctggaacaggaagaggatcagcaactgcgtggccgactacagcgtgctgtacaacagcgccagcttcagcaccttcaagtgctacggcgtgagccccaccaagttaaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcacgaacctcgtgaagaacaagtgcgtgaacttcaacttcaacggcggcagctccggcggctacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaatga

SEQ ID NO: 11 - RNA, 990 nt, synthetic construct
  source 1..990: mol_type = other RNA
atgttcgtgttcctggtgctgctgcccctggtgtcgtcgcagtgcgtgaacctgaccaccggcatctaccagacctcgaacttcagggtgcagcccaccgagtcgatcgtgaggttccccaacatcaccaacctgtgccccttcggcgaggtgttcaacgccaccaggttcgcgtcggtgtacgcctggaacaggaagaggatcagcaactgcgtggccgactactcggtgctgtacaactcggcctcgttctcgaccttcaagtgctacggcgtgtcgcccaccaagttaaacgacctgtgcttcaccaacgtgtacgccgactcgttcgtgatcaggggcgacgaggtgaggcagatcgcccccggccagaccggcaagatcgccgactacaactacaagctgcccgacgacttcaccggctgcgtgatcgcctggaactcgaacaacctggacagcaaggtgggcggcaactacaactacctgtacaggctgttcaggaagtcgaacctgaagcccttcgagagggacatctcgaccgagatctaccaggccggctcgaccccctgcaacggcgtggagggcttcaactgctacttccccctgcagtcgtacggcttccagcccaccaacggcgtgggctaccagccctacagagtggtggtgctgtcgttcgagctgctgcacgcccccgccaccgtgtgcggccccaagaagtcgacgaacctcgtgaagaacaagtgcgtgaacttcaacttcaacggcggctcgtcgggcggctacgagcagtacatcaagtggccctggtacatctggctgggcttcatcgccggcctgatcgccatcgtgatggtgaccatcatgctgtgctgcatgacctcgtgctgctcgtgcctgaagggctgctgctcgtgcggctcgtgctgcaagttcgacgaggacgactcggagcccgtgctgaagggcgtgaagctgcactacacctaatga

SEQ ID NO: 12 - protein, 328 aa, SARS-CoV-2; identical to SEQ ID NO: 3

SEQ ID NO: 13 - RNA, 990 nt, SARS-CoV-2; identical to SEQ ID NO: 10
  source 1..990: mol_type = mRNA

SEQ ID NO: 14 - DNA, 990 bp, synthetic construct
  source 1..990: mol_type = other DNA
atgttcgtgttcctggtgctgctgcccctggtgagcagccagtgcgtgaacctgaccaccggcatctaccagaccagcaacttcagggtgcagcccaccgagagcatcgtgaggttccccaacatcaccaacctgtgccccttcggcgaggtgttcaacgccaccaggttcgcgagcgtgtacgcctggaacaggaagaggatcagcaactgcgtggccgactacagcgtgctgtacaacagcgccagcttcagcaccttcaagtgctacggcgtgagccccaccaagttaaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcacgaacctcgtgaagaacaagtgcgtgaacttcaacttcaacggcggcagctccggcggctacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaatga

SEQ ID NO: 15 - protein, 328 aa, SARS-CoV-2
MFVFLVLLPLVSSQCVNLTTGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGGSSGGYEQYIKWPWYIWLGFIAGLIAIVMVTIMLCCMTSCCSCLKGCCSCGSCCKFDEDDSEPVLKGVKLHYT

SEQ ID NO: 16 - RNA, 990 nt, SARS-CoV-2
  source 1..990: mol_type = mRNA
atgttcgtgttcctggtgctgctgcccctggtgagcagccagtgcgtgaacctgaccaccggcatctaccagaccagcaacttcagggtgcagcccaccgagagcatcgtgaggttccccaacatcaccaacctgtgccccttcggcgaggtgttcaacgccaccaggttcgcgagcgtgtacgcctggaacaggaagaggatcagcaactgcgtggccgactacagcgtgctgtacaacagcgccagcttcagcaccttcaagtgctacggcgtgagccccaccaagttaaacgacctgtgcttcaccaacgtgtacgccgacagcttcgtgatcaggggcgacgaggtgaggcagatcgcccccggccagaccggcaacatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtgaagggcttcaactgctacttccccctgcagagctacggcttccagcccacctacggcgtgggctaccagccctacagagtggtggtgctgagcttcgagctgctgcacgcccccgccaccgtgtgcggccccaagaagagcacgaacctcgtgaagaacaagtgcgtgaacttcaacttcaacggcggcagctccggcggctacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaatga

SEQ ID NO: 17 - DNA, 990 bp, synthetic construct
  source 1..990: mol_type = other DNA
atgttcgtgttcctggtgctgctgcccctggtgagcagccagtgcgtgaacctgaccaccggcatctaccagaccagcaacttcagggtgcagcccaccgagagcatcgtgaggttccccaacatcaccaacctgtgccccttcggcgaggtgttcaacgccaccaggttcgcgagcgtgtacgcctggaacaggaagaggatcagcaactgcgtggccgactacagcgtgctgtacaacagcgccagcttcagcaccttcaagtgctacggcgtgagccccaccaagttaaacgacctgtgcttcaccaacgtgtacgccgacagcttcgtgatcaggggcgacgaggtgaggcagatcgcccccggccagaccggcaacatcgccgactacaactacaagctgcccgacgacttcaccggctgcgtgatcgcctggaacagcaacaacctggacagcaaggtgggcggcaactacaactacctgtacaggctgttcaggaagagcaacctgaagcccttcgagagggacatcagcaccgagatctaccaggccggcagcaccccctgcaacggcgtgaagggcttcaactgctacttccccctgcagagctacggcttccagcccacctacggcgtgggctaccagccctacagagtggtggtgctgagcttcgagctgctgcacgcccccgccaccgtgtgcggccccaagaagagcacgaacctcgtgaagaacaagtgcgtgaacttcaacttcaacggcggcagctccggcggctacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaatga

SEQ ID NO: 18 - protein, 328 aa, SARS-CoV-2
MFVFLVLLPLVSSQCVNLTTGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGGSSGGYEQYIKWPWYIWLGFIAGLIAIVMVTIMLCCMTSCCSCLKGCCSCGSCCKFDEDDSEPVLKGVKLHYT

SEQ ID NO: 19 - RNA, 990 nt, SARS-CoV-2
  source 1..990: mol_type = mRNA
atgttcgtgttcctggtgctgctgcccctggtgagcagccagtgcgtgaacctgaccaccggcatctaccagaccagcaacttcagggtgcagcccaccgagagcatcgtgaggttccccaacatcaccaacctgtgccccttcggcgaggtgttcaacgccaccaggttcgcgagcgtgtacgcctggaacaggaagaggatcagcaactgcgtggccgactacagcgtgctgtacaacagcgccagcttcagcaccttcaagtgctacggcgtgagccccaccaagttaaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaagccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcacgaacctcgtgaagaacaagtgcgtgaacttcaacttcaacggcggcagctccggcggctacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaatga

SEQ ID NO: 20 - DNA, 990 bp, synthetic construct
  source 1..990: mol_type = other DNA
atgttcgtgttcctggtgctgctgcccctggtgagcagccagtgcgtgaacctgaccaccggcatctaccagaccagcaacttcagggtgcagcccaccgagagcatcgtgaggttccccaacatcaccaacctgtgccccttcggcgaggtgttcaacgccaccaggttcgcgagcgtgtacgcctggaacaggaagaggatcagcaactgcgtggccgactacagcgtgctgtacaacagcgccagcttcagcaccttcaagtgctacggcgtgagccccaccaagttaaacgacctgtgcttcaccaacgtgtacgccgacagcttcgtgatcaggggcgacgaggtgaggcagatcgcccccggccagaccggcaagatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaagccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcacgaacctcgtgaagaacaagtgcgtgaacttcaacttcaacggcggcagctccggcggctacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaatga

SEQ ID NO: 21 - protein, 328 aa, SARS-CoV-2
MFVFLVLLPLVSSQCVNLTTGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGGSSGGYEQYIKWPWYIWLGFIAGLIAIVMVTIMLCCMTSCCSCLKGCCSCGSCCKFDEDDSEPVLKGVKLHYT

SEQ ID NO: 22 - RNA, 990 nt, SARS-CoV-2
  source 1..990: mol_type = mRNA
atgttcgtgttcctggtgctgctgcccctggtgagcagccagtgcgtgaacctgaccaccggcatctaccagaccagcaacttcagggtgcagcccaccgagagcatcgtgaggttccccaacatcaccaacctgtgccccttcggcgaggtgttcaacgccaccaggttcgcgagcgtgtacgcctggaacaggaagaggatcagcaactgcgtggccgactacagcgtgctgtacaacagcgccagcttcagcaccttcaagtgctacggcgtgagccccaccaagttaaacgacctgtgcttcaccaacgtgtacgccgacagcttcgtgatcaggggcgacgaggtgaggcagatcgcccccggccagaccggcaacatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaagccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcacgaacctcgtgaagaacaagtgcgtgaacttcaacttcaacggcggcagctccggcggctacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaatga

SEQ ID NO: 23 - DNA, 990 bp, synthetic construct
  source 1..990: mol_type = other DNA
atgttcgtgttcctggtgctgctgcccctggtgagcagccagtgcgtgaacctgaccaccggcatctaccagaccagcaacttcagggtgcagcccaccgagagcatcgtgaggttccccaacatcaccaacctgtgccccttcggcgaggtgttcaacgccaccaggttcgcgagcgtgtacgcctggaacaggaagaggatcagcaactgcgtggccgactacagcgtgctgtacaacagcgccagcttcagcaccttcaagtgctacggcgtgagccccaccaagttaaacgacctgtgcttcaccaacgtgtacgccgacagcttcgtgatcaggggcgacgaggtgaggcagatcgcccccggccagaccggcaacatcgccgactacaactacaagctgcccgacgacttcaccggctgcgtgatcgcctggaacagcaacaacctggacagcaaggtgggcggcaactacaactacaggtacaggctgttcaggaagagcaacctgaagcccttcgagagggacatcagcaccgagatctaccaggccggcagcaagccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcacgaacctcgtgaagaacaagtgcgtgaacttcaacttcaacggcggcagctccggcggctacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaatga

SEQ ID NO: 24 - protein, 328 aa, SARS-CoV-2
MFVFLVLLPLVSSQCVNLTTGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGGSSGGYEQYIKWPWYIWLGFIAGLIAIVMVTIMLCCMTSCCSCLKGCCSCGSCCKFDEDDSEPVLKGVKLHYT

SEQ ID NO: 25 - RNA, 990 nt, SARS-CoV-2
  source 1..990: mol_type = mRNA
atgttcgtgttcctcgtcctcctgcccctggtgtcgtcgcagtgcgtgaacctcaccaccggcatctaccagaccagcaacttccgggtgcagcccacggagagcatcgtccgcttcccgaacatcaccaacctctgccccttcgacgaggtgttcaacgcgacgcgcttcgccagcgtctacgcctggaaccggaagcggatctccaactgcgtcgcggactactccgtcctgtacaacctcgccccattcttcacgttcaagtgctacggggtgtcgccgacgaagctcaacgacctgtgcttcacgaacgtctacgcagactccttcgtcatccggggcgacgaggtgcgccagatcgccccaggacagaccgggaacatcgcggactacaactacaagctccccgacgacttcaccgggtgcgtcatcgcctggaacagcaacaagctcgactcgaaggtctcggggaactacaactacctgtacaggctcttccggaagtcgaacctgaagccattcgagcgcgacatctcgaccgagatctaccaggcgggcaacaagccatgcaacggggtggccggcttcaactgctacttcccgctccgcagctactcgttccgcccaacctacggcgtggggcaccagccgtacagggtggtcgtgctgtccttcgagctgctgcacgcgccggccaccgtctgcggcccaaagaagtcgacgaacctggtcaagaacaagtgcgtcaacttcaacttcaacgggggcagctcgggggggtacgagcagtacatcaagtggccgtggtacatctggctggggttcatcgccgggctcatcgcgatcgtgatggtgacgatcatgctgtgctgcatgaccagctgctgctcgtgcctcaaggggtgctgctcgtgcggatcgtgctgcaagttcgacgaggacgactcggagcccgtcctcaagggggtcaagctccactacacctgataa

SEQ ID NO: 26 - DNA, 990 bp, synthetic construct
  source 1..990: mol_type = other DNA
atgttcgtgttcctcgtcctcctgcccctggtgtcgtcgcagtgcgtgaacctcaccaccggcatctaccagaccagcaacttccgggtgcagcccacggagagcatcgtccgcttcccgaacatcaccaacctctgccccttcgacgaggtgttcaacgcgacgcgcttcgccagcgtctacgcctggaaccggaagcggatctccaactgcgtcgcggactactccgtcctgtacaacctcgccccattcttcacgttcaagtgctacggggtgtcgccgacgaagctcaacgacctgtgcttcacgaacgtctacgcagactccttcgtcatccggggcgacgaggtgcgccagatcgccccaggacagaccgggaacatcgcggactacaactacaagctccccgacgacttcaccgggtgcgtcatcgcctggaacagcaacaagctcgactcgaaggtctcggggaactacaactacctgtacaggctcttccggaagtcgaacctgaagccattcgagcgcgacatctcgaccgagatctaccaggcgggcaacaagccatgcaacggggtggccggcttcaactgctacttcccgctccgcagctactcgttccgcccaacctacggcgtggggcaccagccgtacagggtggtcgtgctgtccttcgagctgctgcacgcgccggccaccgtctgcggcccaaagaagtcgacgaacctggtcaagaacaagtgcgtcaacttcaacttcaacgggggcagctcgggggggtacgagcagtacatcaagtggccgtggtacatctggctggggttcatcgccgggctcatcgcgatcgtgatggtgacgatcatgctgtgctgcatgaccagctgctgctcgtgcctcaaggggtgctgctcgtgcggatcgtgctgcaagttcgacgaggacgactcggagcccgtcctcaagggggtcaagctccactacacctgataa

SEQ ID NO: 27 - protein, 328 aa, SARS-CoV-2
MFVFLVLLPLVSSQCVNLTTGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGGSSGGYEQYIKWPWYIWLGFIAGLIAIVMVTIMLCCMTSCCSCLKGCCSCGSCCKFDEDDSEPVLKGVKLHYT

SEQ ID NO: 28 - RNA, 990 nt, SARS-CoV-2
  source 1..990: mol_type = mRNA
atgttcgtgttcctcgtcctcctgcccctggtgtcgtcgcagtgcgtgaacctcaccaccggcatctaccagaccagcaacttccgggtgcagcccacggagagcatcgtccgcttcccgaacatcaccaacctctgccccttcgacgaggtgttcaacgcgacgcgcttcgccagcgtctacgcctggaaccggaagcggatctccaactgcgtcgcggactactccgtcctgtacaacttcgccccattcttcgcgttcaagtgctacggggtgtcgccgacgaagctcaacgacctgtgcttcacgaacgtctacgcagactccttcgtcatccggggcaacgaggtgagccagatcgccccaggacagaccgggaacatcgcggactacaactacaagctccccgacgacttcaccgggtgcgtcatcgcctggaacagcaacaagctcgactcgaaggtcggggggaactacaactacctgtacaggctcttccggaagtcgaacctgaagccattcgagcgcgacatctcgaccgagatctaccaggcgggcaacaagccatgcaacggggtggccggcttcaactgctacttcccgctccgcagctacgggttccgcccaacctacggcgtggggcaccagccgtacagggtggtcgtgctgtccttcgagctgctgcacgcgccggccaccgtctgcggcccaaagaagtcgacgaacctggtcaagaacaagtgcgtcaacttcaacttcaacgggggcagctcgggggggtacgagcagtacatcaagtggccgtggtacatctggctggggttcatcgccgggctcatcgcgatcgtgatggtgacgatcatgctgtgctgcatgaccagctgctgctcgtgcctcaaggggtgctgctcgtgcggatcgtgctgcaagttcgacgaggacgactcggagcccgtcctcaagggggtcaagctccactacacctgataa

SEQ ID NO: 29 - DNA, 990 bp, synthetic construct
  source 1..990: mol_type = other DNA
atgttcgtgttcctcgtcctcctgcccctggtgtcgtcgcagtgcgtgaacctcaccaccggcatctaccagaccagcaacttccgggtgcagcccacggagagcatcgtccgcttcccgaacatcaccaacctctgccccttcgacgaggtgttcaacgcgacgcgcttcgccagcgtctacgcctggaaccggaagcggatctccaactgcgtcgcggactactccgtcctgtacaacttcgccccattcttcgcgttcaagtgctacggggtgtcgccgacgaagctcaacgacctgtgcttcacgaacgtctacgcagactccttcgtcatccggggcaacgaggtgagccagatcgccccaggacagaccgggaacatcgcggactacaactacaagctccccgacgacttcaccgggtgcgtcatcgcctggaacagcaacaagctcgactcgaaggtcggggggaactacaactacctgtacaggctcttccggaagtcgaacctgaagccattcgagcgcgacatctcgaccgagatctaccaggcgggcaacaagccatgcaacggggtggccggcttcaactgctacttcccgctccgcagctacgggttccgcccaacctacggcgtggggcaccagccgtacagggtggtcgtgctgtccttcgagctgctgcacgcgccggccaccgtctgcggcccaaagaagtcgacgaacctggtcaagaacaagtgcgtcaacttcaacttcaacgggggcagctcgggggggtacgagcagtacatcaagtggccgtggtacatctggctggggttcatcgccgggctcatcgcgatcgtgatggtgacgatcatgctgtgctgcatgaccagctgctgctcgtgcctcaaggggtgctgctcgtgcggatcgtgctgcaagttcgacgaggacgactcggagcccgtcctcaagggggtcaagctccactacacctgataa